(19) United States
(12) Patent Application Publication
(10) Pub. No.: US 2012/0046306 A1
(43) Pub. Date: Feb. 23, 2012

(54) Substituted Heteroaryl Spiropyrrolidine MDM2 Antagonists

(76) Inventors: David Joseph Bartkovitz, Nutley, NJ (US); Xin-Jie Chu, Livingston, NJ (US); Qingjie Ding, Bridgewater, NJ (US); Prabha Saba Karnachi, Hillsborough, NJ (US); Jin-Jun Liu, Warren Township, NJ (US); Sung-Sau So, Verona, NJ (US); Jing Zhang, Parsippany, NJ (US); Zhuming Zhang, Hillsborough, NJ (US)

(21) Appl. No.: 13/180,775

(22) Filed: Jul. 12, 2011

Related U.S. Application Data
(60) Provisional application No. 61/374,725, filed on Aug. 18, 2010.
(60) Provisional application No. 61/494,553, filed on Jun. 8, 2011.

(51) Int. Cl. A61K 31/437; C07D 487/20; A61P 35/00; A61K 31/519; A61K 31/407; C07D 471/20; C07D 495/20

(52) U.S. Cl. 514/265.1; 546/15; 544/230; 548/410; 514/278; 514/409

(57) ABSTRACT

There are provided compounds of the general formula

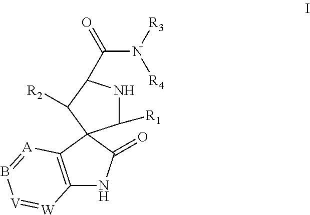

wherein A, B, V, W, R₁, R₂, R₃, R₃, and R₄, are as described herein and enantiomers and pharmaceutically acceptable salts thereof. The compounds are useful as anticancer agents.

### PRIORITY TO RELATED APPLICATION(S)

This application claims the benefit of U.S. Provisional Application No. 61/494,553, filed Jun. 8, 2011, and U.S. Provisional Application No. 61/61/374,725, filed Aug. 18, 2010. The entire contents of the above-identified applications are hereby incorporated by reference.

### FIELD OF THE INVENTION

The present invention relates to heteroaryl spiropyrrolidine derivatives which act as inhibitors of MDM2-p53 interactions and are useful in the amelioration or treatment of cancer.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

### SUMMARY OF THE INVENTION

The present invention relates to heteroaryl spiropyrrolidine I which act as antagonists of mdm2 interactions and hence are useful as potent and selective anticancer agents. The present compounds are of the general formula

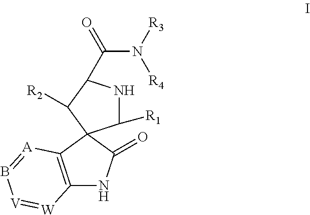

wherein A, B, V, W, R₁, R₂, R₃, R₃, and R₄, are as described herein and enantiomers and pharmaceutically acceptable salts thereof.

### DETAILED DESCRIPTION OF THE INVENTION

There are provided compounds of the formula

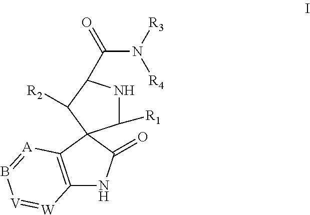

wherein

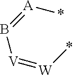

is selected from the group consisting of

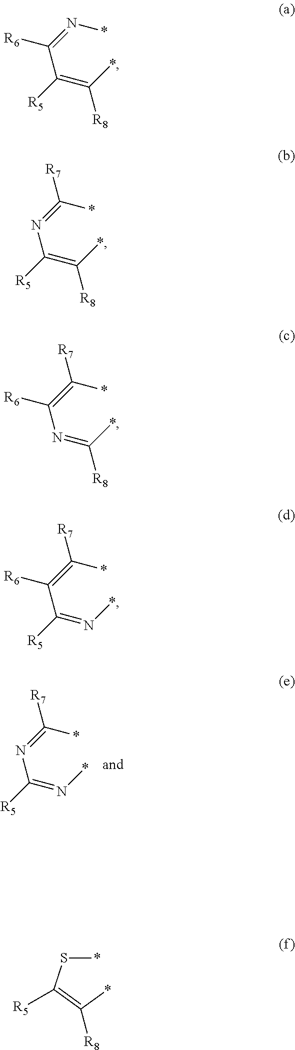

wherein in the case of (f) A is a bond;

R₅ is selected from the group consisting of H, F, Cl, Br, I, cyano, nitro, ethynyl, cyclopropyl, methyl, ethyl, isopropyl, vinyl and methoxy;
R₆ is selected from the group consisting of H, F, Cl, methyl;
R₇ is selected from the group consisting of H, F, Cl, methyl;
R₈ is selected from the group consisting of H, F, Cl, methyl;
R₁ and R₂ are independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₃ and R₄ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6
or a pharmaceutically acceptable salt thereof.

Another embodiment of the invention relates to compounds of formula I having a stereochemical structure shown as formula II

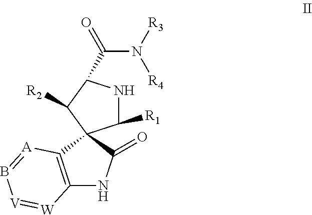

wherein

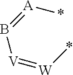

is selected from the group consisting of

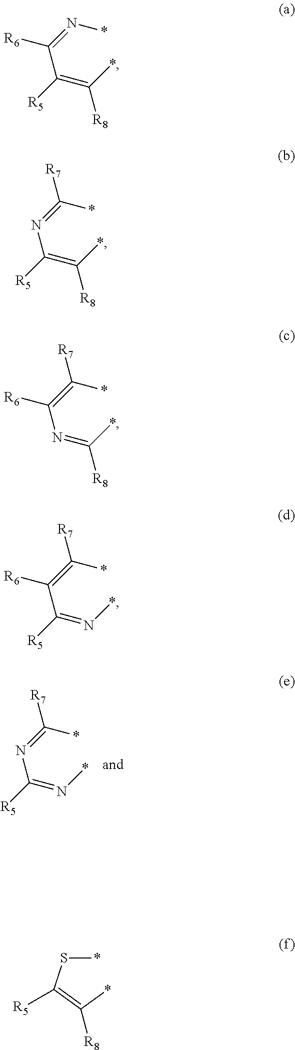

wherein in the case of (f) A is a bond;

R₅ is selected from the group consisting of H, F, Cl, Br, I, cyano, nitro, ethynyl, cyclopropyl, methyl, ethyl, isopropyl, vinyl and methoxy;
R₆ is selected from the group consisting of H, F, Cl and methyl;
R₇ is selected from the group consisting of H, F, Cl and methyl;
R₈ is selected from the group consisting of H, F, Cl and methyl;
R₁ and R₂ are independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ and R₄ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6
or a pharmaceutically acceptable salt thereof.

Preferred are compounds of Formula I, including compounds of Formula II, or a pharmaceutically acceptable salt thereof wherein R₅ is F, Cl or Br.

Preferred are compounds of Formula I, including compounds of Formula II, or a pharmaceutically acceptable salt thereof wherein R₆, R₇, R₈ are all hydrogen.

Preferred are compounds of Formula I, including compounds of Formula II, or a pharmaceutically acceptable salt thereof wherein R₂ is selected from the group consisting of aryl, aryl substitued with Cl, F or Brand heteroaryl optionally substituted with H, F, Cl or Br.

Preferred are compounds of Formula I, including compounds of Formula II, or a pharmaceutically acceptable salt thereof wherein R₁ is a substituted lower alkyl of the formula

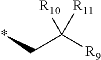

where R₉ and R₁₀ are both methyl, or alternatively, R₉ and R₁₀ together with the carbon to which they are attached form a ring selected from cyclopropyl, cyclobutyl, cyclopentyl or acyclohexyl;

R₁₁ is (CH₂)_{q}—R₁₂, where q is 0, 1 or 2 and

R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle.

Preferred are compounds of Formula I, including compounds of Formula II or a pharmaceutically acceptable salt thereof wherein one of R₃ and R₄ is hydrogen, and the other is (CH₂)ₙ—R′, n is 0 or 1 and R′ is aryl, substituted aryl, hetereoaryl, substituted heteroaryl, hetereocycle or substituted heterocycle.

Preferred are compounds of Formula I, including compounds of Formula II, or a pharmaceutically acceptable salt thereof wherein

R₅ is selected from F, Cl or Br;
R₆, R₇, R₈ are hydrogen;
R₂ is selected from the group consisting of aryl, aryl substitued with Cl or F or Br, and heteroaryl optionally substituted with H, F, Cl or Br;
R₁ is a substituted lower alkyl of the formula

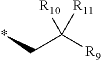

where R₉ and R₁₀ are both methyl, or alternatively, R₉ and R₁₀ together with the carbon to which they are attached form a ring selected from cyclopropyl, cyclobutyl, cyclopentyl or acyclohexyl;
R₁₁ is (CH₂)_{q}—R₁₂, where q is 0, 1 or 2;
R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
one of R₃ and R₄ is hydrogen, and the other is (CH₂)ₙ—R′;
n is 0 or 1 and
R′ is selected from aryl, substituted aryl, hetereoaryl, substituted heteroaryl, hetereocycle or substituted heterocycle.

Further preferred are compounds of Formula II

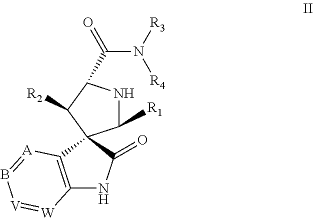

wherein

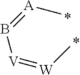

is selected from the group consisting of

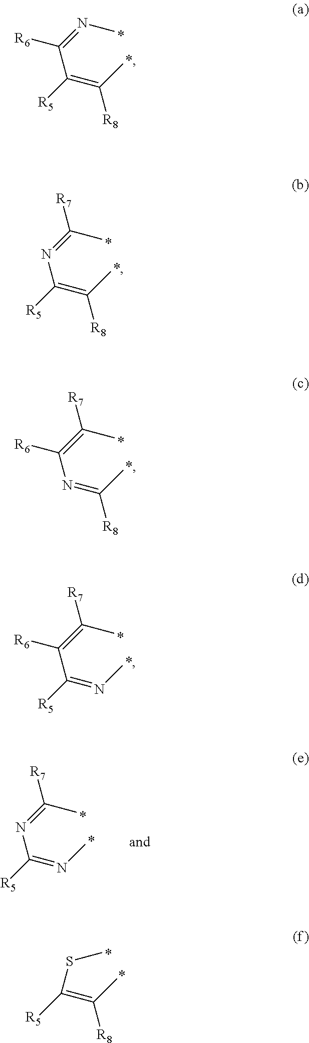

R₅ is selected from F, Cl or Br;
R₆, R₇, R₈ are hydrogen;
R₂ is selected from the group consisting of

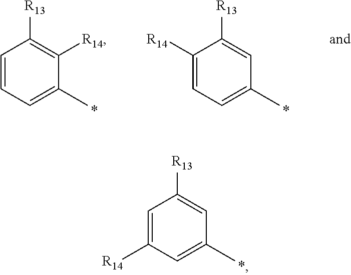

wherein

### R₁₃ is F, Cl or Br;

### R₁₄ is H or F;

R₁ is a substituted lower alkyl of the formula

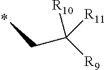

where R₉ and R₁₀ are both methyl, or alternatively, R₉ and R₁₀ together with the carbon to which they are attached form a ring selected from cyclopropyl, cyclobutyl, cyclopentyl or acyclohexyl;
R₁₁ is (CH₂)_{q}—R₁₂, where q is 0, 1 or 2;
R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
one of R₃ and R₄ is hydrogen, and the other is (CH₂)ₙ—R′;
n is 0 or 1;
R′ is selected from aryl, substituted aryl, hetereoaryl, substituted heteroaryl, hetereocycle or substituted heterocycle
or a pharmaceutically acceptable salt thereof.

Especially preferred are compounds of the formula
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate, rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
rac-(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid;
rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoic acid;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid;
rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[2,3-b]pyridine]-5-carboxamide;
methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoic acid;
methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoate;
rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoic acid;
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid;
chiral methyl 4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
chiral 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
rac-(2S,3S,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid;
chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate;
chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate;
chiral 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
chiral 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2R,3S,4R,5S)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
chiral 4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
chiral 4-((2R,3R,4R,5S)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
methyl rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoate;
rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoic acid;
rac-(2S,3S,4R,5R)-6′-chloro-4-(3-chlorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4R,5R)-N-(4-carbamoyl-2-methoxyphenyl)-6′-chloro-4-(3-chlorophenyl)-2-neopentyl-2′-oxo-1′,2′-dihydrospiro[pyrrolidine-3,3′-pyrrolo[3,2-b]pyridine]-5-carboxamide;
methyl rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate;
methyl rac-4-{(2S,3R,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate;
rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoic acid;
rac-(2S,3S,4S,5R)-4-(3-bromo-2-fluorophenyl)-6′-chloro-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-bromo-2-fluorophenyl)-N-(4-carbamoyl-2-methoxyphenyl)-6′-chloro-2-neopentyl-2′-oxo-1′,2′-dihydrospiro[pyrrolidine-3,3′-pyrrolo[3,2-b]pyridine]-5-carboxamide;
rac-2-(4-((2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido)-3-methoxyphenoxy)ethyl acetate;
rac-(2S,3S,4R,5R)-6′-chloro-4-(3-chlorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-bromo-2-fluorophenyl)-6′-chloro-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
chiral(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
rac-(2S,3S,4S,5R)-6′-bromo-4-(3-chloro-2-fluorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-6′-bromo-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-6′-bromo-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoic acid;
rac-(2S,3S,4S,5R)-4-(3-chloro-2-fluorophenyl)-N-(4-cyano-2-methoxyphenyl)-6′-fluoro-2-neopentyl-2′-oxo-1′,2′-dihydrospiro[pyrrolidine-3,3′-pyrrolo[3,2-b]pyridine]-5-carboxamide;
rac-(2S,3S,4S,5R)-N-(4-carbamoyl-2-methoxyphenyl)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-chloro-2-fluorophenyl)-N-[4-(2-hydroxyethoxy)-2-methoxyphenyl]-6′-fluoro-2-neopentyl-2′-oxo-1′,2′-dihydrospiro[pyrrolidine-3,3′-pyrrolo[3,2-b]pyridine]-5-carboxamide;
rac-(2S,3R,4S,5R)-2′-chloro-4-(3-chloro-2-fluorophenyl)-N-[4-(2-hydroxyethoxy)-2-methoxyphenyl]-2-neopentyl-6′-oxo-6′,7′-dihydrospiro[pyrrolidine-3,5′-pyrrolo[2,3-d]pyrimidine]-5-carboxamide;
methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate;
methyl rac-4-{(2S,3S,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate;
rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoic acid;
rac-(2S,3R,4S,5R)-N-(4-carbamoyl-2-methoxyphenyl)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-carboxamide;
methyl rac-4-((2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate;
methyl rac-4-((2S,3S,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate;
rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid;
chiral 4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid;
rac-(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
chiral(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
rac-(2S,3S,4R,5R)-2′-chloro-4-(3-chloro-2-fluorophenyl)-N-(4-cyano-2-methoxyphenyl)-2-neopentyl-5′-oxo-4′,5′-dihydrospiro[pyrrolidine-3,6′-thieno[3,2-b]pyrrole]-5-carboxamide;
rac-(2S,3S,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
rac-(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide,
chiral(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide,
methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoic acid,
methyl rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoic acid,
rac-(2S,3R,4S,5R)-N-(4-carbamoyl-2-methoxyphenyl)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-carboxamide,
rac-2-(4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxyphenoxy)ethyl acetate,
rac-(2S,3R,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-c]pyridine]-5-carboxamide,
methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoic acid,
rac-(2S,3R,4S,5R)-N-(4-carbamoyl-2-methoxyphenyl)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-carboxamide,
methyl rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate,
rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid and
rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-carboxamide.

In the specification where indicated the various groups may be substituted by 1-5 or, preferably, 1-3 substituents independently selected from the group consisting of lower alkyl, lower-alkenyl, lower-alkynyl, dioxo-lower-alkylene (forming e.g. a benzodioxyl group), halogen, hydroxy, CN, CF₃, NH₂, N(H, lower-alkyl), N(lower-alkyl)₂, aminocarbonyl, carboxy, NO₂, lower-alkoxy, thio-lower-alkoxy, lower-alkylsulfonyl, aminosulfonyl, lower-alkylcarbonyl, lower-alkylcarbonyloxy, lower-alkoxycarbonyl, lower-alkyl-carbonyl-NH, fluoro-lower-alkyl, fluoro-lower-alkoxy, lower-alkoxy-carbonyl-lower-alkoxy, carboxy-lower-alkoxy, carbamoyl-lower-alkoxy, hydroxy-lower-alkoxy, NH₂-lower-alkoxy, N(H, lower-alkyl)-lower-alkoxy, N(lower-alkyl)₂-lower-alkoxy, lower-alkyl-1-oxiranyl-lower-alkoxy-lower-alkyl, 2-oxo-pyrrolidin-1-yl, (1,1-dioxo)-2-isothiazolidine, 3-lower-alkyl sulfinyl, a substituted or unsubstituted heterocyclic ring, a substituted or unsubstituted aryl ring, a substituted or unsubstituted heteroaryl ring, trifluoro-lower-alkylsulfonylamino-aryl, lower-alkyl sulfonylaminocarbonyl, lower-alkyl sulfonylaminocarbonyl-aryl, hydroxycarbamoyl-phenyl, benzyloxy-lower-alkoxy, mono- or di-lower alkyl substituted amino-sulfonyl and lower-alkyl which can optionally be substituted with halogen, hydroxy, NH₂, N(H, lower-alkyl) or N(lower-alkyl)₂. Preferred substituents for the cycloalkyl, cycloalkenyl, aryl, heteroaryl and heterocycle rings are halogen, lower alkoxy, lower alkyl, hydroxycarbonyl, carboxy, carboxy lower alkoxy, oxo and CN. Preferred substituents for alkyl are alkoxy and N(lower alkyl)₂.

### DEFINITIONS

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkoxy, alkoxyl or lower alkoxy” refers to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Amino means the group —NH₂.

“Aryl” means a monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “halogen” as used herein means fluorine, chlorine, bromine, or iodine, preferably fluorine and chlorine.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl.

In the case of aryl or heteroaryl which are bicyclic it should be understood that one ring may be aryl while the other is heteroaryl and both being substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

Hydroxy or hydroxyl is a prefix indicating the presence of a monovalent —OH group.

“IC50” refers to the concentration of a particular compound required to inhibit 50% of a specific measured activity. IC₅₀ can be measured, inter alia, as is described subsequently in the Example providing biological data.

“Lower” as in “lower alkenyl” means a group having 1 to 6 carbon atoms.

“Nitro” means —NO₂.

Oxo means the group ═O.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (I.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent.

### Synthetic Methods

The present invention provides novel methods for the synthesis of heteroaryl spiropyrrolidines of formula I or II. Compounds of this invention can be synthesized according to the following general schemes. Suitable processes for synthesizing these compounds are provided in the examples.

An intermediate III can be made from a base-catalyzed condensation reaction of appropriately selected substituted 4- or 5- or 6- or 7-aza-2-oxindole I and appropriate substituted aldehyde II in methanol (Scheme 1). The choice of bases includes but is not limited to pyrrolidine or piperidine. The reaction generates III as a mixture of Z- and E-isomers with E-isomer as the major product.

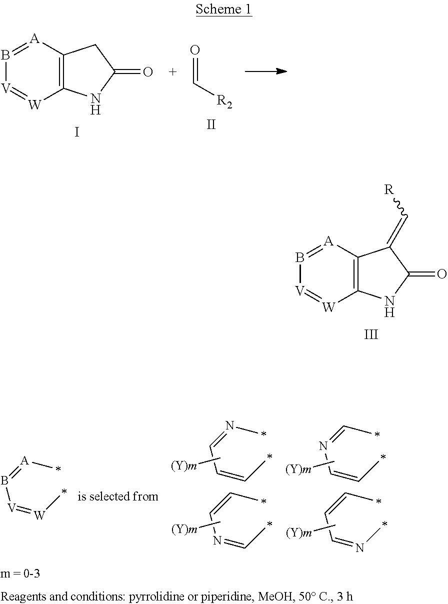

Similarly, intermediates V or VII can be made from a acid-catalyzed condensation reaction of appropriately selected substituted 5,7-dihydro-pyrrolo[2,3-d]pyrimidin-6-one IV or 4,6-dihydro-thieno[3,2-b]pyrrol-5-one VI and aldehyde II in hydrochloric and acetic acid (M. Cheung et al, Tetrahedron Lett. 2001, 42, 999) (Scheme 2).

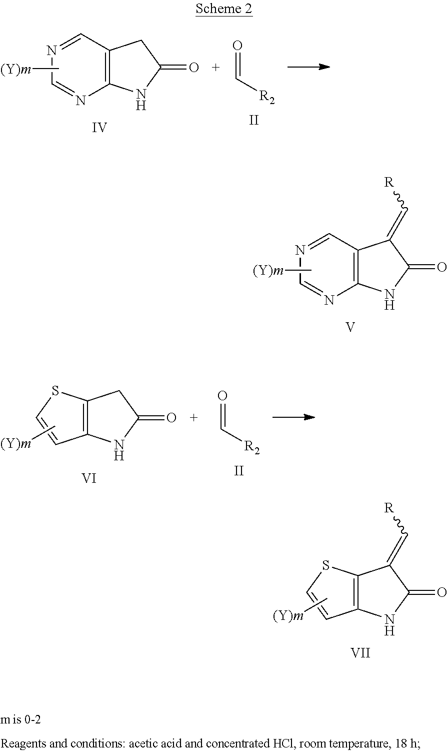

Preparation of starting material VIa is described in Scheme 3 to exemplify the synthesis of intermediate VI in Scheme 2. 2,5-dichlorothiophene can be treated with sodium nitrate in concentrated sulfuric acid to give 2,5-dichlorothiphene-3-nitrothiophene. Nucleophilic substitution of 5-chloro group with tert-butyl ethyl malonate mono-sodium salt and treatment with trifluoroacetic acid lead to methyl 2-(5-chloro-3-nitrothiophen-2-yl)acetate (WO2008132139). Reduction of nitro group with Zinc and ammonium chloride and cyclizing reaction to form amide promoted by trimathylaluminum afford intermediate VIa (S. Hu, et al, J. Heterocyclic. Chem. 2005, 42, 661).

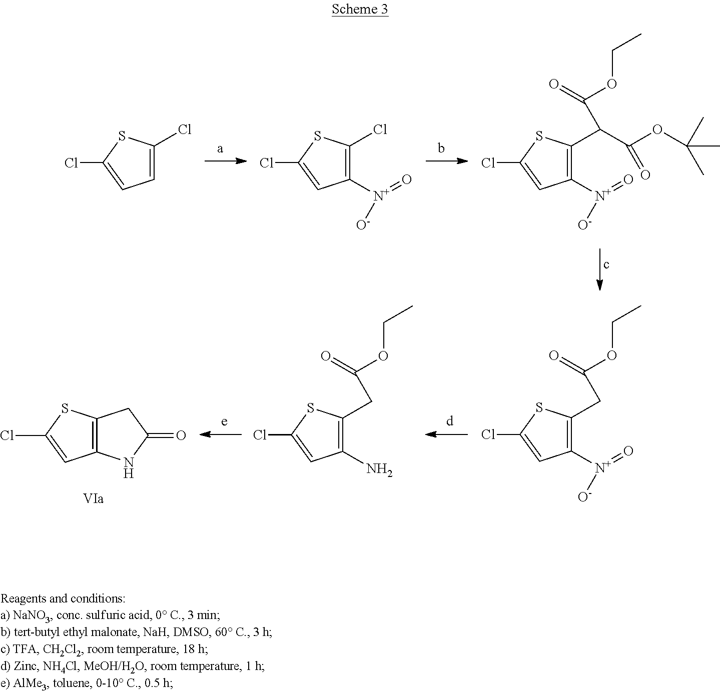

Racemic synthesis of compounds in formula I and II can be achieved as outlined in Scheme 4. Amine NHR₃R₄ can be reacted with N-protected glycine like N-Boc glycine by using a coupling reagent like EDCI or HATU to give intermediate VIII. Intermediate VIII can be treated with trifluoroacetic acid or HCl at room temperature to remove protective Boc group and give intermediate IX. Appropriately selected aldehyde R₁CHO can react with IX to give the imine X. The cycloaddition reaction between intermediates X and intermediate III or V or VII mediated by LiOH or LiCl/DABCO gives a racemic and diastereomeric mixture of compounds XI in formula I together with other isomers. Compounds XI can be purified by flash chromatography followed by chiral separation by chiral Super Fluid Chromatography (SFC) or chiral HPLC to give optically pure or enriched chiral compounds XII in formula II.

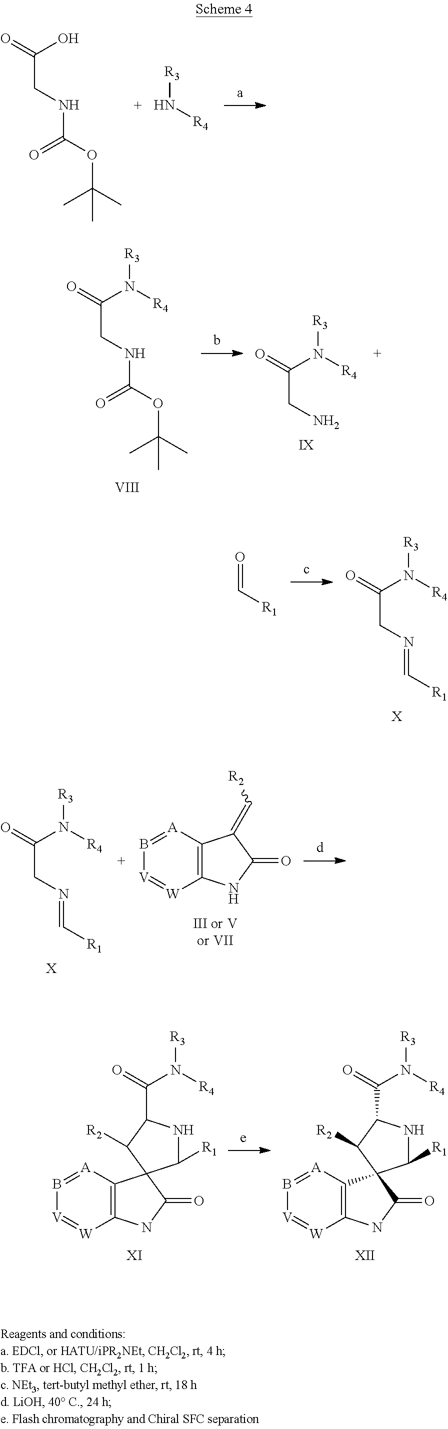

Similarly, compounds in formula I and II can be prepared as outlined in Scheme 5. Intermediate III or V or VII can be protected with Boc group to give intermediate XIII. The cycloaddition reaction between intermediates X and XIII mediated by LiOH or LiCl/DABCO follow by reaction to remove Boc group by trifluoroacetic acid give compounds XI in formula I. Compounds XI can be subsequently separated into optically pure or enriched chiral compounds XII in formula II.

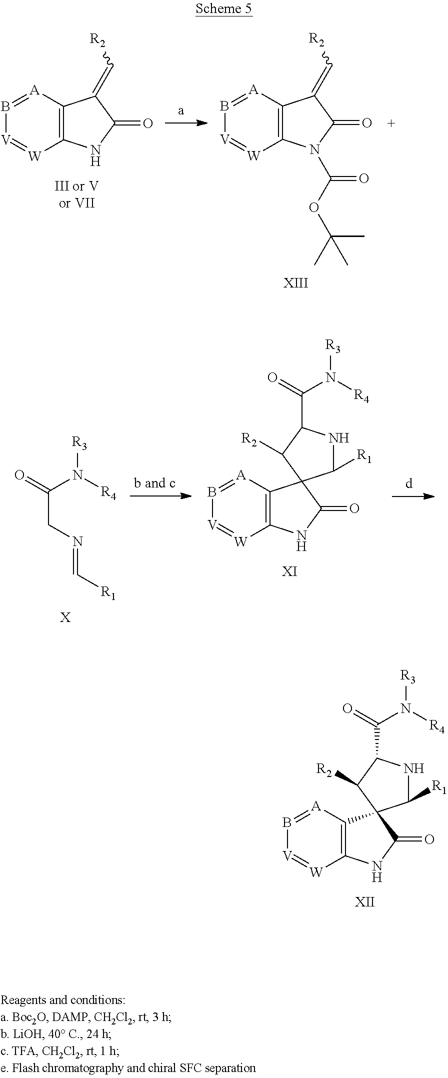

Alternative synthesis of compounds XIIa in formula II can be achieved. As illustrated in Scheme 6, selected aldehyde R₁CHO can be reacted with glycine tert-butyl ester to generate imine XIV. The racemic mixture of intermediate XV and XV′ can be made from intermediates XIV and VII by LiOH mediated cyclization reaction. The mixture of XVI and XVI′ can be subsequently converted to a racemic mixture of acid XVII and XVII′ by using trifluoroacetic acid. Amide formation with various amine NHR₃R₄ by using diphenylphsphinic chloride as the coupling reagent can lead to the racemic mixture of compounds XIIa and XIIa′ in formula II. Finally chiral separation by chiral Super Fluid Chromatography (SFC) or chiral HPLC gives optically pure or enriched chiral compounds XIIa in formula II.

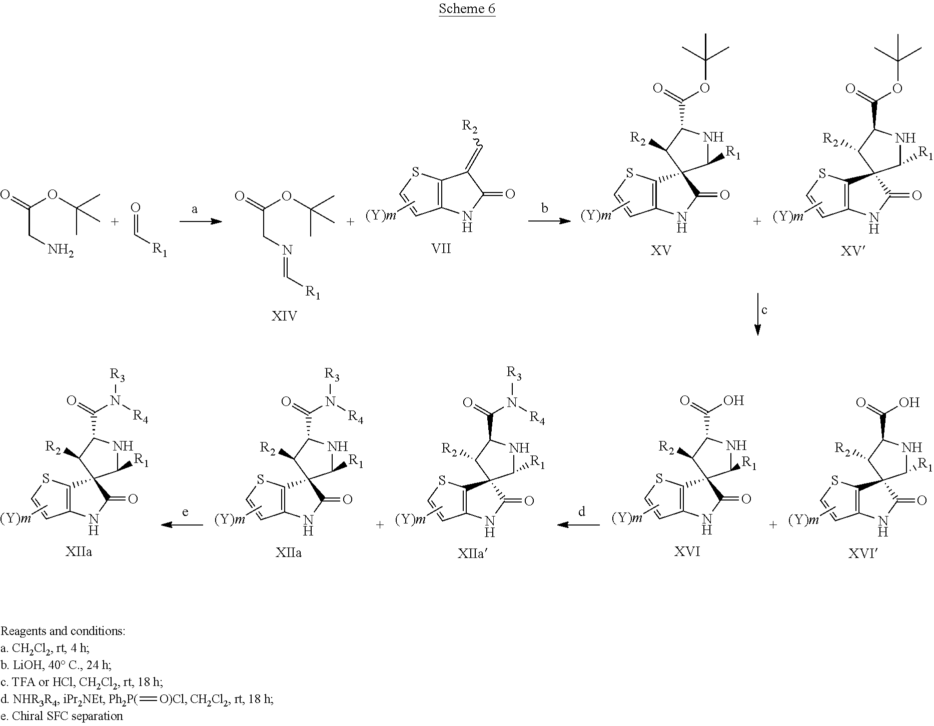

### EXAMPLES

The compounds of the present invention may be synthesized according to novel techniques. The following examples and references are provided to aid the understanding of the present invention, the true scope of which is set forth in the appended claims.

### Example 1

### Preparation of intermediate (E)-methyl 4-(2-(3,3-dimethylbutylideneamino)acetamido)benzoate

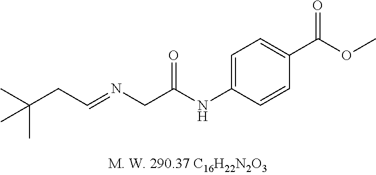

Step a: A mixture of methyl 4-aminobenzoate (Aldrich, 5.00 g, 32.4 mmol), tert-butoxycarbonylamino-acetic acid (9.44 g, 53.4 mmol, Aldrich) and 1-(3-dimetgylaminopropyl)-3-ethylcarbodiimide hydrochloride (Chem-Impex, 10.16 g, 53.1 mmol) in CH₂Cl₂ (60 mL) was stirred vigorously at rt for 2 h. The reaction mixture was then concentrated in vacuum and the residue was dissolved in EtOAc (150 mL), washed successively with water (120 mL), sat. NH₄Cl (20 mL), sat. NaHCO₃ (50 mL), water (50 mL) and brine (20 mL), dried over Na₂SO₄ and concentrated in vacuum. The white solid was further dried in vacuum overnight to give methyl 4-(2-(tert-butoxycarbonylamino)acetamido)-benzoate (11.28 g).

Step b: A solution of methyl 4-(2-(tert-butoxycarbonylamino)acetamido)-benzoate (5.25 g, 17.0 mmol) in CH₂Cl₂ (40 mL) at 0 C. was treated with TFA (20 mL) and the mixture was stirred at rt for 4 h. The solvent was then removed under reduced pressure. The residue was further dried in vacuum overnight to give methyl 4-(2-aminoacetamido)benzoate as a TFA salt (5.82 g, 99%).

Step c: To a suspension of the above methyl 4-(2-aminoacetamido)benzoate TFA salt (4.95 g, 15.4 mmol) in t-butyl methyl ether (160 mL) at rt was added TRIETHYLAMINE (1.74 g, 2.40 ml, 17.2 mmol) and the mixture was stirred for 30 min. 3,3-Dimethylbutanal (1.69 g, 16.9 mmol) in t-butyl methyl ether (5 mL) was added and the reaction mixture was allowed to stir at rt overnight. t-Butyl methyl ether (100 mL) was added and stirred for 20 min. The solid was filtrated off and the filtrate was washed with water, brine, dried over Na₂SO₄ and concentrated to give (E)-methyl 4-(2-(3,3-dimethylbutylideneamino)acetamido)-benzoate as a white solid (3.25 g, 72%). MS (ES⁺) m/z [(M+H)⁺]: 291

### Example 2

### Preparation of intermediate 4-{2-[3,3-Dimethyl-but-(E)-ylideneamino]-acetylamino}-2-methoxybenzoic acid methyl ester

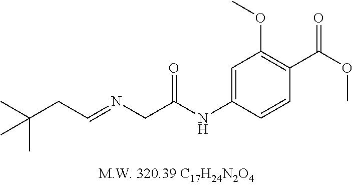

To a suspension of methyl 4-(2-aminoacetamido)-2-methoxybenzoate hydrochloride (prepared in a similar manner as described in Example 1 (0.50 g, 1.82 mmol) in t-butyl methyl ether (8 mL) at rt, was added triethylamine (203 mg, 2.01 mmol) and the mixture was stirred for 30 min. 3,3-Dimethylbutanal (0.24 g, 0.30 mL, 2.27 mmol, Aldrich) in t-butyl methyl ether (2 mL) was added and the reaction mixture was allowed to stir at rt overnight. t-Butyl methyl ether (50 mL) was added and stirred for 20 min. The solid was filtrated off and the filtrate was washed with water, brine, dried over Na₂SO₄ and concentrated to give 4-{2-[3,3-dimethyl-but-(E)-ylideneamino]-acetylamino}-2-methoxybenzoic acid methyl ester as a white solid (353 mg, 60%). MS (ES⁺) m/z [(M+H)⁺]: 320

### Example 3

### Preparation of intermediate (E)-methyl 4-(2-(3,3-dimethylbutylideneamino)acetamido)-3-methoxybenzoate

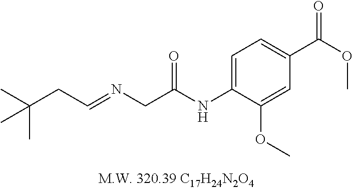

(E)-methyl 4-(2-(3,3-dimethylbutylideneamino)acetamido)-3-methoxybenzoate was prepared in a manner similar to the method described in Example 1. MS (ES⁺) m/z [(M+H)⁺]: 320

### Example 4

### Preparation of intermediate E/Z-3-(3-Chloro-2-fluoro-benzylidene)-1,3-dihydro-pyrrolo[3,2c]pyridin-2-one

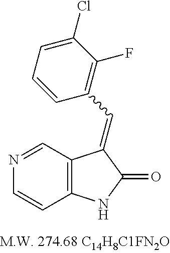

To a suspension of 1H-pyrrolo[3,2-c]pyridin-2(3H)-one (1.00 g, 7.46 mmol, prepared according to the method described in J. Org. Chem., 1991, 56, 4805-4808) in MeOH (55 mL) in a 100-mL round-bottomed flask, was added 3-chloro-2-fluorobenzaldehyde (Oakwood Products, 3.55 g, 22.40 mmol), giving a clear solution. Piperidine (Lancaster, 2.66 g, 31.20 mmol) was added slowly. After stirred for 10 min, the reaction mixture was heated at 50° C. for 3 h, resulting in a yellow precipitation. The reaction mixture was cooled and the precipitate was filtered, washed with cold MeOH and dried in vacuum to give E/Z-3-(3-chloro-2-fluoro-benzylidene)-1,3-dihydro-pyrrolo[3,2-c]pyridin-2-one as a light yellow crystalline solid (1.38 g, 67%). MS (ES⁺) m/z [(M+H)⁺]: 275

### Example 5

### Preparation of intermediate rac-(2S,3S,4S,5R)-tert-butyl 4-(3-chloro-2-fluorophenyl)-2-neopentyl-2′-oxo-1′,2′-dihydrospiro[pyrrolidine-3,3′-pyrrolo[3,2-c]pyridine]-5-carboxylate

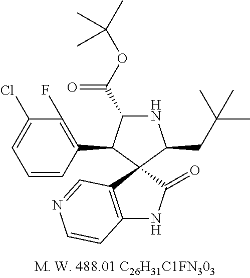

Step a: To a solution of tert-butyl 2-aminoacetate (Aldrich, 1.00 g, 7.62 mmol) in CH₂Cl₂ (30 mL) was added 3,3-dimethylbutanal (Aldrich, 1.00 g, 9.98 mmol). The mixture was stirred at rt for 4 h. Water added and organic layer separated. The aqueous layer was extracted with CH₂Cl₂. The combined organic extracts were washed with water and concentrated to give (E)-tert-butyl 2-(3,3-dimethylbutylideneamino)acetate as a colorless oil (1.52 g, 93%).

Step b: A suspension of 3-(3-chloro-2-fluorobenzylidene)-1H-pyrrolo[3,2-c]pyridin-2(3H)-one (60 mg, 0.218 mmol, Example 4) in CH₂Cl₂ (8 mL) was treated with triethylamine (133 mg, 1.31 mmol). (E)-tert-butyl 2-(3,3-dimethylbutylideneamino)acetate (69 mg, 0.325 mmol) was added followed by silver(I) fluoride (Aldrich, 47 mg, 0.370 mmol) and the mixture was stirred at rt for 24 h. The reaction mixture was partition between EtOAc and water, washed with brine and dried over Na₂SO₄ and concentrated to dryness. The residue was dissolved in t-BuOH (8 mL) and 2,3,4,6,7,8,9,10-octahydropyrimido[1,2-a]azepine (DBU) (Aldrich, 266 mg, 1.75 mmol) and heated at 120 C. for 2 h. The reaction mixture was then cooled to rt and partitioned between EtOAc and water, washed with water, brine and concentrated. The crude material was purified by flash chromatography (EtOAc/CH₂Cl₂: 5/95 to 40/60) to give rac-(2S,3S,4S,5R)-tert-butyl 4-(3-chloro-2-fluorophenyl)-2-neopentyl-2′-oxo-1′,2′-dihydrospiro[pyrrolidine-3,3′-pyrrolo[3,2-c]pyridine]-5-carboxylate (10.2 mg, 8.6%) as a white solid. MS (ES⁺) m/z [(M+H)⁺]: 488

### Example 6

### Preparation of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate

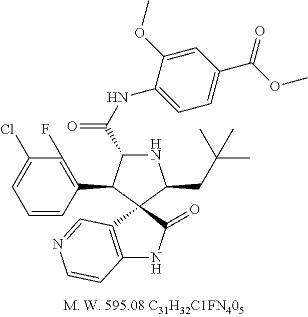

To a suspension of 3-(3-chloro-2-fluorobenzylidene)-1H-pyrrolo[3,2-c]pyridin-2(3H)-one (450 mg, 1.64 mmol, Example 4) in anhydrous THF (25 mL) at 40° C., was added anhydrous LiOH (19 mg, 0.82 mmol) and the suspension was stirred at 40° C. for 10 min before (E)-methyl 4-(2-(3,3-dimethylbutylideneamino)-acetamido)-3-methoxybenzoate (551 mg, 1.72 mmol, Example 3) was added in one portion. The reaction mixture was allowed to stir at 40° C. for 23 h. The mixture was diluted with EtOAc (100 mL), washed with water (2×20 mL) and concentrated to a small volume. MeOH was added slowly (˜15 mL) and the mixture was stirred in cold bath for 20 min. The resulting precipitate was filtered, washed with cold MeOH and dried in vacuum overnight to give methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate as a white powder (556 mg, 56%). MS (ES⁺) m/z [(M+H)⁺]: 595

### Example 7

### Preparation of rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid

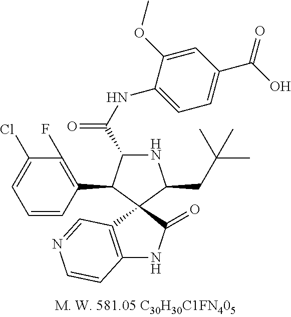

To a solution of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (151 mg, 0.254 mmol, Example 6) in THF (8 mL) was added a solution of LiOH hydrate (63 mg, 1.50 mmol) in water (4 mL). The reaction mixture was stirred at rt for 20 h before it was treated with 1N HCl to slightly acidic. The mixture was partitioned between CH₂Cl₂ (30 mL) and water (10 mL), extracted with CH₂Cl₂ (3×30 mL). The combined organic extracts were washed with water, dried over Na₂SO₄, concentrated and lyophized to give rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white powder (145 mg, 95%). MS (ES⁺) m/z [(M+H)⁺]: 581

### Example 8

### Preparation of rac-(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

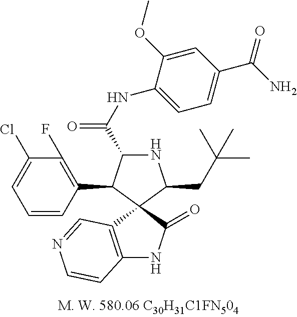

A mixture of rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid (51 mg, 0.087 mmol, Example 7) and 1,1′-carbonyldiimidazole (Aldrich, 28 mg, 0.174 mmol) in THF (3 mL) was stirred at rt for 17 h. Ammonium hydroxide (180 mg, 5.14 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 3/97) to give rac-(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (25 mg, 50%). MS (ES⁺) m/z [(M+H)⁺]: 580

### Example 9

### Preparation of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate

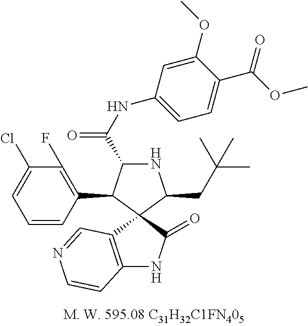

To a solution of E/Z-(E)-3-(3-chloro-2-fluorobenzylidene)-1H-pyrrolo[3,2-c]pyridin-2(3H)-one (173 mg, 0.63 mmol, Example 4) in anhydrous THF (12 mL) was added anhydrous LiOH (11 mg, 0.43 mmol) and the mixture was stirred at 40° C. for 10 min. (E)-methyl 4-(2-(3,3-dimethylbutylideneamino)acetamido)-2-methoxybenzoate (220 mg, 0.68 mmol, Example 2) was added in one portion. The reaction mixture was allowed to stir at 40° C. overnight, giving a clear reaction mixture. This mixture was diluted with EtOAc and washed with water, brine and dried over Na₂SO₄ and concentrated. The crude product was purified on flash chromatography (EtOAc/CH₂Cl₂, 3/97 to 60/40) to give methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate as a white solid (119 mg, 32%). MS (ES⁺) m/z [(M+H)⁺]: 595

### Example 10

### Preparation of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid

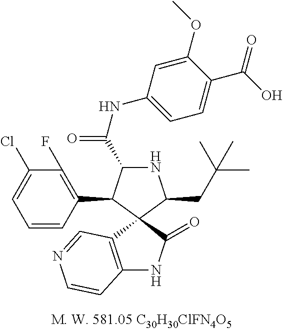

To a suspension of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate (107 mg, 0.181 mmol, Example 9) in THF (10 mL) was added a solution of LiOH hydrate (61 mg, 1.47 mmol) in water (5 mL). The reaction mixture was stirred at rt overnight until the reaction was complete. The reaction mixture was then treated with 1N HCl to slightly acidic, diluted with ethyl acetate (100 mL), washed with water (10 mL), dried with Na₂SO₄ and concentrated to give rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid as a white solid (89 mg, 85%). MS (ES⁺) m/z [(M+H)⁺]: 581

### Example 11

### Preparation of rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

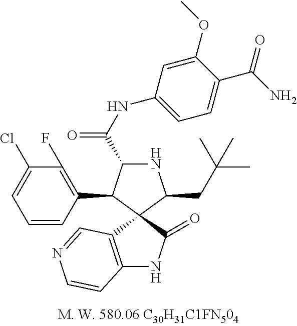

A mixture of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid (30 mg, 0.052 mmol, Example 10) and 1,1′-carbonyldiimidazole (Aldrich, 37 mg, 0.22 mmol) in THF (3 mL) was stirred at rt for 17 hr. Ammonium hydroxide (180 mg, 5.14 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 3/97) to give rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (27 mg, 89%). MS (ES⁺) m/z [(M+H)⁺]: 580

### Example 12

### Preparation of intermediate E/Z-3-(3-Chloro-2-fluoro-benzylidene)-1,3-dihydro-pyrrolo[2,3b]pyridin-2-one

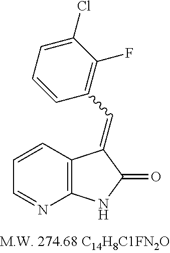

To a suspension of 1H-pyrrolo[2,3-b]pyridin-2(3H)-one (Chemgenx, 926 mg, 6.91 mmol) in MeOH (55 mL), was added 3-chloro-2-fluorobenzaldehyde (Oakwood Products, 3.29 g, 20.70 mmol). Piperidine (Lancaster, 2.41 g, 2.8 mL, 28.30 mmol) was added slowly. After stirring a few minutes, the reaction mixture was heated at 50° C. for 3 h, resulting in a yellow precipitation. The reaction mixture was cooled and the precipitate was filtered, washed with cold MeOH and dried in vacuum to give E/Z-3-(3-chloro-2-fluoro-benzylidene)-1,3-dihydro-pyrrolo[2,3-b]pyridin-2-one as a light yellow crystalline solid (1.18 g, 62%). MS (ES⁺) m/z [(M+H)⁺]: 275

### Example 13

### Preparation of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate

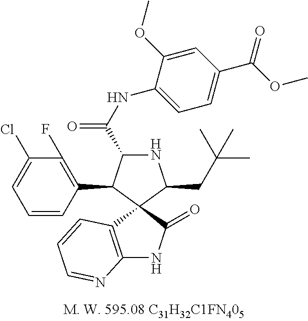

To a suspension of E/Z-3-(3-chloro-2-fluoro-benzylidene)-1,3-dihydro-pyrrolo[2,3-b]pyridin-2-one (401 mg, 1.46 mmol, Example 12) in anhydrous THF (25 mL) at 40° C. was added anhydrous LiOH (17 mg, 0.73 mmol) and the suspension was stirred at 40° C. for 10 min before (E)-methyl 4-(2-(3,3-dimethylbutylideneamino)acetamido)-3-methoxybenzoate (490 mg, 1.53 mmol, Example 3) was added in one portion. The reaction mixture was allowed to stir at 40° C. for 23 h. The mixture was diluted with EtOAc, washed with water and concentrated to a small volume. MeOH was added slowly (˜15 mL) and the mixture was stirred in cold bath for ˜20 min. The resulting precipitate was filtered, washed with cold MeOH and dried overnight in vacuum to give methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate as a white solid (543 mg, 62%). MS (ES⁺) m/z [(M+H)⁺]: 595

### Example 14

### Preparation of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid

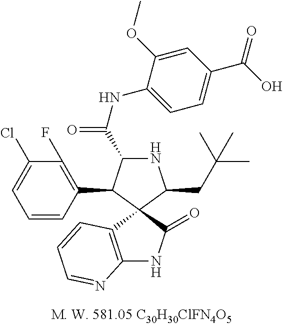

To a solution of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate (154 mg, 0.26 mmol, Example 13) in THF (8 mL) was added a solution of LiOH hydrate (77 mg, 1.84 mmol) in water (4 mL). The reaction mixture was stirred at rt for 20 h before it was treated with 1N HCl to slightly acidic. The mixture was partitioned between CH₂Cl₂ (30 mL) and water (10 ml), extracted with CH₂Cl₂. The combined organic extracts were washed with water, dried over Na₂SO₄, concentrated and lyophized to give rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white solid (150 mg, 97%). MS (ES⁺) m/z [(M+H)⁺]: 581

### Example 15

### Preparation of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate

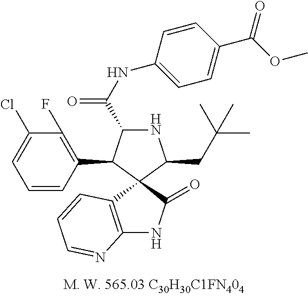

To a suspension of 3-(3-chloro-2-fluorobenzylidene)-1H-pyrrolo[2,3-b]pyridin-2(3H)-one (102 mg, 0.372 mmol, Example 12) in anhydrous THF (25 mL) at 40° C. was added hydrous LiOH (5 mg, 0.21 mmol) and the suspension was stirred at 40° C. for 10 min before (E)-methyl 4-(2-(3,3-dimethylbutylideneamino)acetamido)benzoate (114 mg, 0.391 mmol, Example 1) was added in one portion. The reaction mixture was allowed to stir at 40° C. for 23 h. The mixture was diluted with EtOAc (100 mL) and washed with water (2×20 mL), concentrated to a small volume (˜5 mL). MeOH was added (2 mL) and the solution was stirred in cold bath until precipitate started to form. The precipitate was filtered and washed with cold MeOH, dried overnight in vacuum to give methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate as (47.6 mg, 23%) a white powder. MS (ES⁺) m/z [(M+H)⁺]: 565.

### Example 16

### Preparation of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoic acid

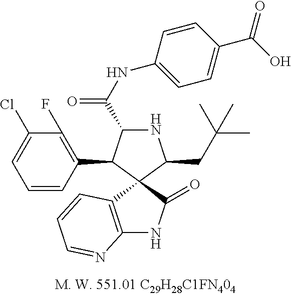

To a solution of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate (45 mg, 0.079 mmol, Example 15) in THF (4 mL) was added a solution of LiOH hydrate (23 mg, 0.55 mmol) in water (2 mL). The reaction mixture was stirred at rt for 24 hrs before it was treated with 1N HCl to slightly acidic. The mixture was partitioned between water (10 mL) and CH₂Cl₂ (30 mL), extracted with CH₂Cl₂. The combined organic extracts were washed with water, dried over Na₂SO₄, concentrated and lyophized to give rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoic acid as a white solid (25 mg, 59%). MS (ES⁺) m/z [(M+H)⁺]: 551

### Example 17

### Preparation of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate

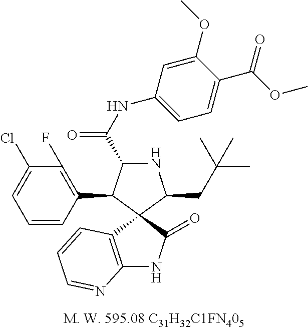

To a solution of E/Z-3-(3-chloro-2-fluorobenzylidene)-1H-pyrrolo[2,3-b]pyridin-2(3H)-one (99 mg, 0.36 mmol, Example 12) in anhydrous THF (9 mL) was added anhydrous LiOH (6 mg, 0.26 mmol) and the mixture was stirred at 40° C. for 10 min. (E)-Methyl 4-(2-(3,3-dimethylbutylideneamino)acetamido)-2-methoxybenzoate (123 mg, 0.385 mmol, Example 2) was added in one portion. The reaction mixture was allowed to stir at 40° C. overnight, giving a clear reaction mixture. This mixture was diluted with EtOAc (100 mL) and washed with water, brine and dried over Na₂SO₄ and concentrated. The crude product was purified by flash chromatography (EtOAc/CH₂Cl₂, 3/97 to 60/40) to give methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate as a white solid (128 mg, 60%). MS (ES⁺) m/z [(M+H)⁺]: 595

### Example 18

### Preparation of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid

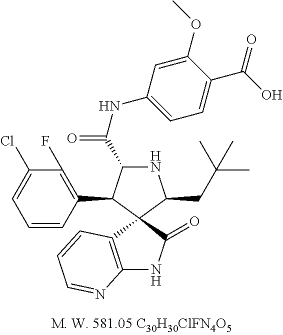

To a suspension of methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate (116 mg, 0.196 mmol, Example 17) in THF (10 mL) was added a solution of LiOH hydrate (66 mg, 1.57 mmol) in water (5 mL). The reaction mixture was stirred at rt overnight and was then treated with 1N HCl to slightly acidic, diluted with ethyl acetate (100 mL), washed with water (10 mL), dried with Na₂SO₄ and concentrated to give rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid as a white solid (103 mg, 91%). MS (ES⁺) m/z [(M+H)⁺]: 581

### Example 19

### Preparation of rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[2,3-b]pyridine]-5-carboxamide

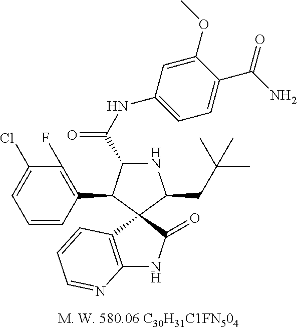

A mixture of rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid (29 mg, 0.051 mmol, Example 18) and 1,1′-carbonyldiimidazole (Aldrich, 35 mg, 0.217 mmol) in THF (3 mL) was stirred at rt for 17 h. Ammonium hydroxide (180 mg, 5.14 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 5/95) to give rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[2,3-b]pyridine]-5-carboxamide as a white solid (23 mg, 78%). MS (ES⁺) m/z [(M+H)⁺]: 580

### Example 20

### Preparation of intermediate E/Z-3-(3-Chloro-2-fluoro-benzylidene)-1,3-dihydro-pyrrolo[3,2-b]pyridin-2-one

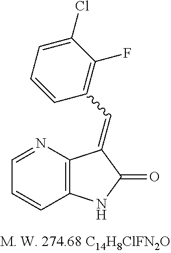

To a suspension of 1H-pyrrolo[3,2-b]pyridin-2(3H)-one (Sinova. 933 mg, 6.96 mmol) in MeOH (35 mL) was added 3-chloro-2-fluorobenzaldehyde (Oakwood, 3.58 g, 22.6 mmol) giving a clear solution. Piperidine (Lancaster, 2.58 g, 30.30 mmol) was added slowly. After stirred for a few minutes, the reaction mixture was heated at 50° C. for 3 h, resulting in a yellow precipitation. The reaction mixture was cooled and the precipitate was filtered, washed with cold MeOH and dried in vacuum to give E/Z-3-(3-chloro-2-fluoro-benzylidene)-1,3-dihydro-pyrrolo[3,2-b]pyridin-2-one as a light yellow crystalline solid (1.22 g, 64%). MS (ES⁺) m/z [(M+H)⁺]: 275

### Example 21

### Preparation of methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate

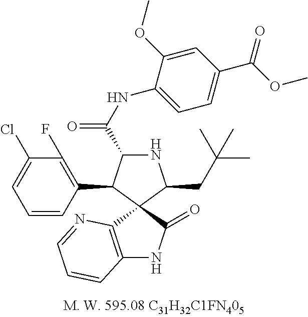

To a solution of E/Z-3-(3-chloro-2-fluoro-benzylidene)-1,3-dihydro-pyrrolo[3,2-b]pyridin-2-one (Example 20, 301 mg, 1.10 mmol) in anhydrous THF (22 mL) at 40° C. was added anhydrous LiOH (13 mg, 0.54 mmol) and the mixture was stirred at 40° C. for 10 min before (E)-methyl 4-(2-(3,3-dimethylbutylideneamino)-acetamido)-3-methoxybenzoate (Example 3, 367 mg, 1.15 mmol) was added in one portion. The reaction mixture was allowed to stir at 40° C. for 20 h, giving a white precipitate. The resulting precipitate was filtered, washed with cold MeOH and dried overnight in vacuum to give methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (242 mg, 37%). MS (ES⁺) m/z [(M+H)⁺]: 595

### Example 22

### Preparation of rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid

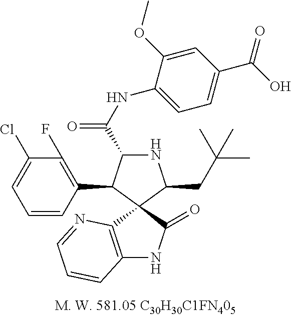

To a suspension of methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (Example 21, 124 mg, 0.21 mmol) in THF (8 mL) was added a solution of LiOH hydrate (69 mg, 1.66 mmol) in water (4 mL). The reaction mixture was stirred at 40° C. for 24 h until the reaction was complete. The reaction mixture was treated with 1N HCl to slightly acidic and the precipitate was filtered and washed with cold MeOH and dried overnight to give rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (114 mg, 94%). MS (ES⁺) m/z [(M+H)⁺]: 581

### Example 23

### Preparation of intermediate E/Z-6-Chloro-3-(3-chloro-2-fluoro-benzylidene)-1,3-dihydro-pyrrolo[3,2-c]pyridin-2-one

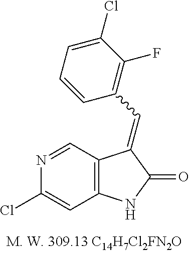

To a suspension of 6-chloro-1H-pyrrolo[3,2-c]pyridin-2(3H)-one (Sinova, 689 mg, 4.09 mmol) in MeOH (50 mL) was added 3-chloro-2-fluorobenzaldehyde (Oakwood, 1.89 g, 11.9 mmol). Piperidine (1.46 g, 17.2 mmol) was added slowly, giving a clear brown solution. After stirred for a few minutes, the reaction mixture was heated at 50° C. for 5 h, resulting in a yellow precipitation. The reaction mixture was cooled and the precipitate was filtered, washed with cold MeOH and dried in vacuum to give E/Z-6-chloro-3-(3-chloro-2-fluoro-benzylidene)-1,3-dihydro-pyrrolo[3,2-c]pyridin-2-one as a light brown solid (1.07 g, 85%). MS (ES⁺) m/z [(M+H)⁺]: 309

### Example 24

### Preparation of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate

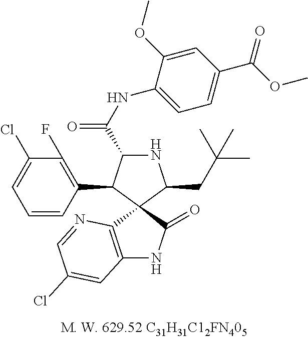

A suspension of E/Z-6-chloro-3-(3-chloro-2-fluoro-benzylidene)-1,3-dihydro-pyrrolo[3,2-c]pyridin-2-one (Example 23, 240 mg, 0.78 mmol) in anhydrous THF (25 mL) was mildly warmed to a clear solution and then cooled down to 40° C. Anhydrous LiOH (8.7 mg, 0.367 mmol) was added and the mixture was stirred at 40° C. for 10 min. (E)-methyl 4-(2-(3,3-dimethylbutylideneamino)-acetamido)-3-methoxybenzoate (Example 3, 235 mg, 0.733 mmol) was added in one portion. The reaction mixture was allowed to stir at 40° C. for 20 h, giving a clear reaction mixture. This mixture was diluted with EtOAc (100 mL) and washed with water, brine and dried over Na₂SO₄ and concentrated. The crude product was purified on flash chromatography (THF/hexane, 5/95 to 50/50) to give methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate as a white solid (210 mg, 45%). MS (ES⁺) m/z [(M+H)⁺]: 629

### Example 25

### Preparation of rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid

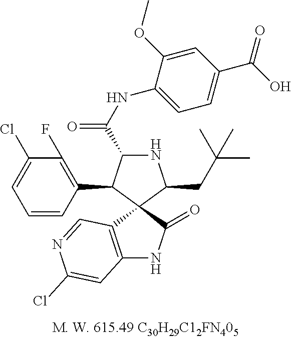

To a suspension of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (Example 24, 48 mg, 0.076 mmol) in THF (4 mL) was added a solution of liOH hydrate (25 mg, 0.60 mmol) in water (2 mL) and the reaction mixture was stirred at rt overnight. The mixture was treated with 1N HCl to slightly acidic, diluted with ethyl acetate (200 mL), washed with water (15 mL), dried with Na₂SO₄ and concentrated to give rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white solid (38 mg, 82%). MS (ES⁺) m/z [(M+H)⁺]: 615

### Example 26

### Preparation of rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

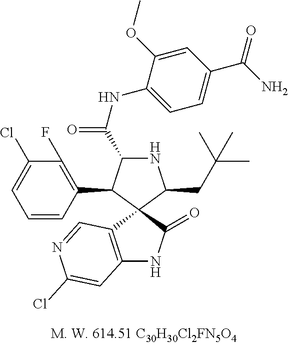

A mixture of rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid (Example 25, 19 mg, 0.031 mmol), N,N-diisopropylethylamine (30 mg, 0.23 mmol) and O-(7-azabenzotriazol-1-yl)-N,N,N′,N′-tetramethyluronium hexafluorophosphate (HATU (Chem-Impex, 22 mg, 0.057 mmol) in DMF (4 mL) was stirred for 2 min before a solution of ammonia in isopropanol (Aldrich, 2 M, 0.08 mL, 0.16 mmol) was added. The mixture was stirred for 0.5 h and poured into EtOAc (70 mL), washed with water (10 mL), brine (15 mL) and concentrated. The crude product was purified by flash chromatography (EtOH/CH₂Cl₂, 0.5/99.5 to 5/95) to give rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (7.6 mg, 37%). MS (ES⁺) m/z [(M+H)⁺]: 614

### Example 27

### Preparation of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate

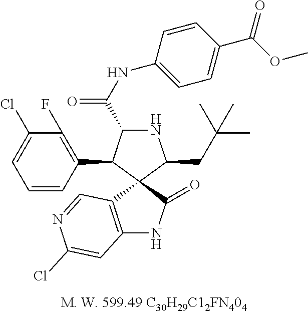

A suspension of E/Z-6-chloro-3-(3-chloro-2-fluorobenzylidene)-1H-pyrrolo[3,2-c]pyridin-2(3H)-one (Example 23, 258 mg, 0.83 mmol) in anhydrous THF (20 mL) was warmed to a clear solution and then cooled down to 40° C. Anhydrous LiOH (10 mg, 0.42 mmol) was added and the mixture was stirred at 40° C. for 10 min. (E)-Methyl 4-(2-(3,3-dimethylbutylideneamino)acetamido)benzoate (Example 1, 247 mg, 0.85 mmol) was added in one portion. The reaction mixture was allowed to stir at 40° C. for 20 h, giving a clear reaction mixture. This mixture was diluted with EtOAc (100 mL) and washed with water, brine and dried over Na₂SO₄ and concentrated. The crude product was purified on flash chromatography (THF/hexane, 5/95 to 50/50) to give methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate as a white solid (148 mg, 29%). MS (ES⁺) m/z [(M+H)⁺]: 599

### Example 28

### Preparation of rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoic acid

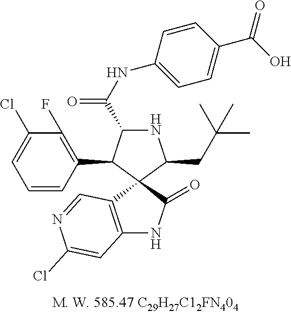

To a suspension of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate (Example 27, 22 mg, 0.036 mmol) in THF (4 mL) was added a solution of LiOH hydrate (25 mg, 0.61 mmol) in water (2 mL) and the reaction mixture was stirred at rt overnight. The reaction mixture was treated with 1N HCl to slightly acidic, diluted with ethyl acetate (100 mL), washed with water (15 mL), dried with Na₂SO₄ and concentrated to give rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoic acid as a white solid (20 mg, 93%). MS (ES⁺) m/z [(M+H)⁺]: 585

### Example 29

### Preparation of intermediate E/Z-3-(3-chloro-2-fluoro-benzylidene)-6-methyl-1,3-dihydro-pyrrolo[3,2-b]pyridin-2-one

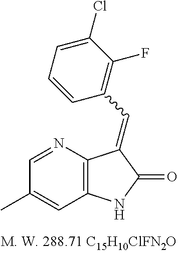

To a suspension of 6-methyl-1H-pyrrolo[3,2-b]pyridin-2(3H)-one (Sinova, 500 mg, 3.37 mmol) in MeOH (20 mL) was added 3-chloro-2-fluorobenzaldehyde (Oakwood, 1.60 g, 10.1 mmol) giving a clear solution. Piperidine (Lancaster, 1.2 g, 14.1 mmol) was added slowly. After stirred for a few minutes, the reaction mixture was heated at 50° C. for 4 h, resulting in a yellow precipitation. The reaction mixture was cooled and the precipitate was filtered, washed with cold MeOH and dried in vacuum to give E/Z-3-(3-chloro-2-fluoro-benzylidene)-6-methyl-1,3-dihydro-pyrrolo[3,2-b]pyridin-2-one as a light yellow crystalline solid (946 mg, 97%). MS (ES⁺) m/z [(M+H)⁺]: 289

### Example 30

### Preparation of methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoate

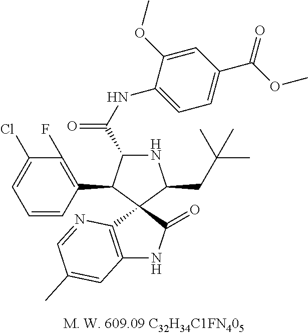

To a suspension of E/Z-3-(3-chloro-2-fluoro-benzylidene)-6-methyl-1,3-dihydro-pyrrolo[3,2-b]pyridin-2-one (Example 29, 300 mg, 1.04 mmol) in anhydrous THF (20 mL) at 40° C. was added anhydrous LiOH (13 mg, 0.55 mmol) and the mixture was stirred at 40° C. for 10 min before (E)-methyl 4-(2-(3,3-dimethylbutylideneamino)acetamido)-3-methoxybenzoate (Example 3, 364 mg, 1.14 mmol) was added in one portion. The reaction mixture was allowed to stir at 40° C. for 23 h, giving a white precipitate. The resulting precipitate was filtered, washed with cold THF and dried overnight in vacuum to give methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (305 mg, 48%). MS (ES⁺) m/z [(M+H)⁺]: 609

### Example 31

### Preparation of rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid

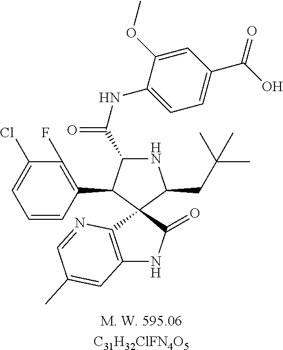

To a suspension of methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoate (Example 30, 288.8 mg, 0.474 mmol) in THF (16 mL) was added a solution of LiOH hydrate (159 mg, 3.79 mmol) in water (8 mL). The reaction mixture was heated at 45° C. for 22 h. After cooled to rt, the mixture was treated with 1N HCl to slightly acidic. The resulting precipitate was filtered, washed with cold water and then THF, dried overnight to give rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid (265 mg, 94%). MS (ES⁺) m/z [(M+H)⁺]: 595

### Example 32

### Preparation of methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoate

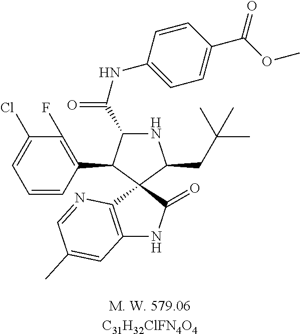

To a suspension of E/Z-3-(3-Chloro-2-fluoro-benzylidene)-6-methyl-1,3-dihydro-pyrrolo[3,2-b]pyridin-2-one (Example 29, 201 mg, 0.69 mmol) in anhydrous THF (15 mL) at 40° C. was added LiOH (9.5 mg, 0.397 mmol) and the mixture was stirred at 40° C. for 10 min before methyl 4-(2-(3,3-dimethylbutylideneamino)-acetamido)benzoate (Example 1, 215 mg, 0.740 mmol) was added in one portion. The reaction mixture was allowed to stir at 40° C. for 21 h, giving a white precipitate. The resulting precipitate was filtered, washed with cold THF and dried overnight in vacuum to give methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoate as a white solid (135 mg, 33%). MS (ES⁺) m/z [(M+H)⁺]: 579

### Example 33

### Preparation of rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoic acid

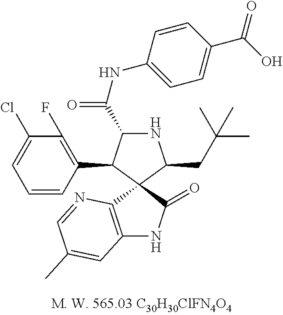

To a suspension of methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoate (Example 32, 109 mg, 0.19 mmol) in THF (12 mL) was added a solution of LiOH hydrate (63 mg, 1.51 mmol) in water (6 mL). The suspension was warmed at 45° C. for 20 h. After cooled to rt, the mixture was treated with 1N HCl to slightly acidic. The resulting precipitate was filtered and washed with cold water and then THF, dried overnight to give rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoic acid (110 mg, 90%). MS (ES⁺) m/z [(M+H)⁺]: 565

### Example 34

### Preparation of intermediate E/Z-6-Chloro-3-(3-chloro-2-fluoro-benzylidene)-1,3-dihydro-pyrrolo[2,3-b]pyridin-2-one

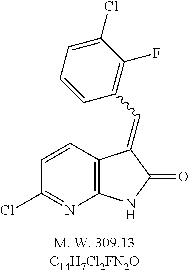

To a suspension of 6-chloro-1,3-dihydro-pyrrolo[2,3-b]pyridin-2-one (Sinova, 501.5 mg, 2.83 mmol) in MeOH (25 mL) was added 3-chloro-2-fluorobenzaldehyde (Oakwood, 1.35 g, 8.51 mmol). Piperidine (Aldrich, 1.03 g, 12.0 mmol) was added slowly, giving a clear brown solution. After stirred for a few minutes, the reaction mixture was heated at 50° C. overnight, resulting in a yellow precipitation. The reaction mixture was cooled and the precipitate was filtered, washed with cold MeOH and dried in vacuum to give E/Z-6-chloro-3-(3-chloro-2-fluoro-benzylidene)-1,3-dihydro-pyrrolo[2,3-b]pyridin-2-one as a grey solid (457 mg, 52%). MS (ES⁺) m/z [(M+H)⁺]: 309

### Example 35

### Preparation of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate

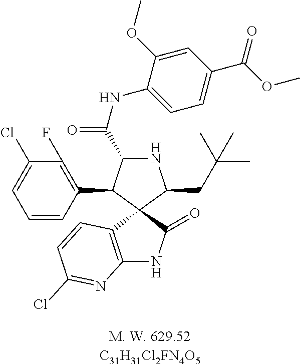

To a solution of E/Z-6-chloro-3-(3-chloro-2-fluoro-benzylidene)-1,3-dihydro-pyrrolo[2,3-b]pyridin-2-one (Example 34, 151 mg, 0.49 mmol) in anhydrous THF (12 mL) was added anhydrous LiOH (9 mg, 0.38 mmol) and the mixture was stirred at 40° C. for 10 min. (E)-methyl 4-(2-(3,3-dimethylbutylideneamino)-acetamido)-3-methoxybenzoate (Example 3, 169 mg, 0.53 mmol) was added in one portion. The reaction mixture was allowed to stir at 40° C. overnight, giving a clear reaction mixture. This mixture was diluted with EtOAc (100 mL) and washed with water, brine and dried over Na₂SO₄ and concentrated. The crude product was purified by flash chromatography (EtOAc/CH₂Cl₂, 1/99 to 50/50) to give methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate as a white solid (34 mg, 11%). MS (ES⁺) m/z [(M+H)⁺]: 629

### Example 36

### Preparation of rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid

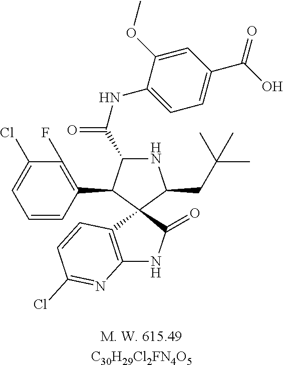

To a suspension of methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate (Example 35, 29 mg, 0.046 mmol) in THF (2 mL) was added a solution of LiOH hydrate (16 mg, 0.39 mmol) in water (2 mL). The reaction mixture was stirred at rt overnight until the reaction was complete. The reaction mixture was treated with 1N HCl to slightly acidic, diluted with ethyl acetate (100 mL), washed with water (10 mL), dried with Na₂SO₄ and concentrated to give rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid as a white solid (26 mg, 92%). MS (ES⁺) m/z [(M+H)⁺]: 615

### Example 37

### Preparation of chiral methyl 4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate (A)

### and chiral methyl 4-((2R,3S,4R,5S)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate(B)

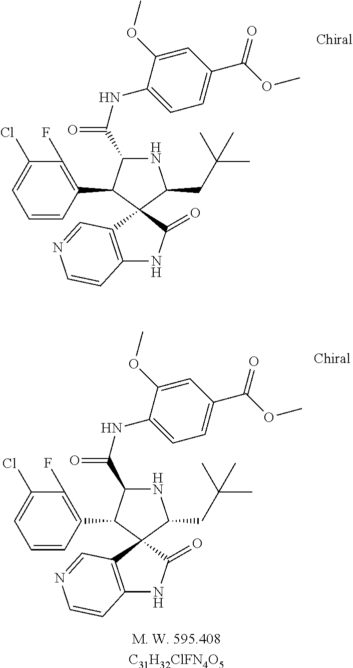

Methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate (310 mg, Example 6) was separated by SFC (Waters/Thar Multi-Gram II, Kromasil 5-CelluCoat OD 3×25 cm., 35° C. at 100 bar, eluting with 40% MeOH in carbon dioxide) to give chiral methyl 4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate, MS (ES⁺) m/z [(M+H)⁺]: 595, and chiral methyl 4-((2R,3S,4R,5S)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate. MS (ES⁺) m/z [(M+H)⁺]: 595

### Example 38

### Preparation of chiral 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid

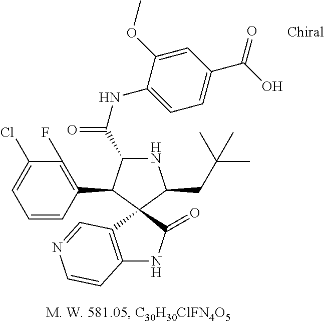

To a solution of chiral methyl 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (Example 37, 83 mg, 0.140 mol) in THF (8 mL) was added a solution of LiOH hydrate (47 mg, 1.14 mmol) in water (4 mL). The reaction mixture was stirred at rt overnight before it was treated with 1N HCl to slightly acidic. The mixture was partitioned between water (10 mL) and EtOAc (100 mL), washed with water, brine and dried over Na₂SO₄, concentrated and lyophized to give chiral 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white powder (53 mg, 66%). MS (ES⁺) m/z [(M+H)⁺]: 581

### Example 39

### Preparation of chiral(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

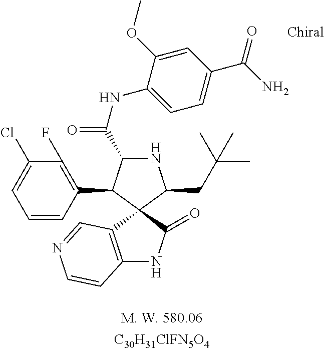

A mixture of chiral 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid (Example 38, 40 mg, 0.069 mmol) and 1,1′-carbonyldiimidazole (Aldrich, 27.6 mg, 0.170 mmol) in THF (3 mL) was stirred at rt for 17 h. Ammonium hydroxide (Aldrich, 153 mg, 4.37 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 3/97) to give chiral(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (37 mg, 92%). MS (ES⁺) m/z [(M+H)⁺]: 580

### Example 40

### Preparation of intermediate E/Z-6-chloro-3-(3-chloro-2-fluoro-benzylidene)-1,3-dihydro-pyrrolo[3,2-b]pyridin-2-one

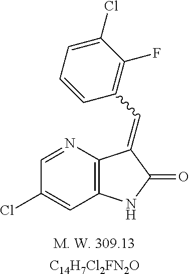

To a mixture of 6-chloro-4-aza-2-oxindole (Sinova, 1.5 g, 8.9 mmol) and 3-chloro-2-fluorobenzaldehyde (Oakwood, 1.4 g, 8.9 mmol) in methanol (65 mL) was added piperidine (Aldrich, 0.76 g, 8.9 mmol) dropwise. The reaction mixture was heated at 50° C. and stirred for 3 h. Then the mixture was cooled to room temperature and filtered. The resulting precipitate was collected and dried to give E/Z-6-chloro-3-(3-chloro-2-fluoro-benzylidene)-1,3-dihydro-pyrrolo[3,2-b]pyridin-2-one as a yellow solid (1.8 g, 65%).

### Example 41

### Preparation of intermediate rac-(2S,3S,4S,5R)-tert-butyl 6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxylate

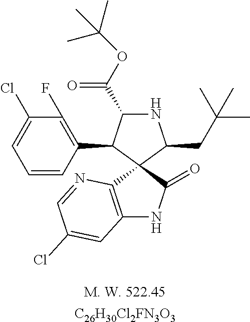

To a suspension of E/Z-6-chloro-3-(3-chloro-2-fluoro-benzylidene)-1,3-dihydro-pyrrolo[3,2-b]pyridin-2-one (Example 40, 623 mg, 2.02 mmol) in anhydrous THF (20 mL) were added 1,4-diazabicyclo[2,2,2]octane (249 mg, 2.22 mmol, Aldrich) and anhydrous LiCl (Aldrich, 101 mg, 2.38 mmol) under N₂. The suspension was warmed at 40° C. for a few minutes before (E)-tert-butyl 2-(3,3-dimethylbutylideneamino)acetate (Example 5, Step a, 522 mg, 2.45 mmol) in CH₂Cl₂ (5 mL) was added. The reaction mixture was allowed to stir at 40° C. for 19 h. The reaction mixture was partitioned between CH₂Cl₂ and water. The organic layer was separated and the aqueous layer was extracted with CH₂Cl₂ twice. The combined extracts were washed with water, brine and evaporated. The crude product was purified by flash chromatography (EtOAc/hexane, 20/80 to 50/50) to give rac-(2S,3S,4S,5R)-tert-butyl 6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxylate as a light yellow solid (261 mg, 25%). MS (ES⁺) m/z [(M+H)⁺]: 522

### Example 42

### Preparation of methyl rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate

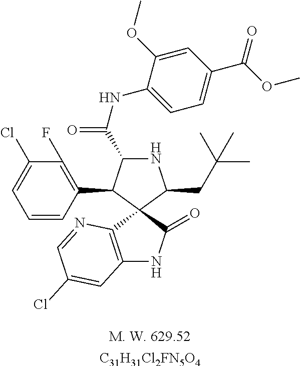

To a solution of E/Z-6-chloro-3-(3-chloro-2-fluorobenzylidene)-1H-pyrrolo[3,2-b]pyridin-2(3H)-one (Example 40, 90 mg, 0.29 mmol) in anhydrous THF (8 mL) was added anhydrous LiOH (5 mg, 0.20 mmol) and the mixture was stirred at 40° C. for 10 min. (E)-Methyl 4-(2-(3,3-dimethylbutylideneamino)acetamido)-3-methoxybenzoate (99 mg, 0.31 mmol) was added in one portion. The reaction mixture was allowed to stir at 40° C. overnight giving a white precipitate. The resulting precipitate was filtered, washed with cold THF and dried overnight in vacuum to give methyl rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (33 mg, 18%). MS (ES⁺) m/z [(M+H)⁺]: 629

### Example 43

### Preparation of rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid

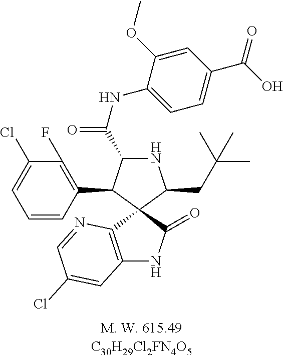

To a suspension of methyl rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (Example 42, 62 mg, 0.10 mmol) in THF (5 mL) was added a solution of LiOH hydrate (34 mg, 0.82 mmol) in water (2.5 mL). The reaction mixture was stirred at rt overnight until the reaction was complete. The mixture was then treated with 1N HCl to slightly acidic, diluted with ethyl acetate (100 mL), washed with water (10 mL), dried with Na₂SO₄ and concentrated to give rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (57 mg, 94%). MS (ES⁺) m/z [(M+H)⁺]:

### Example 44

### Preparation of rac-(2S,3S,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxamide

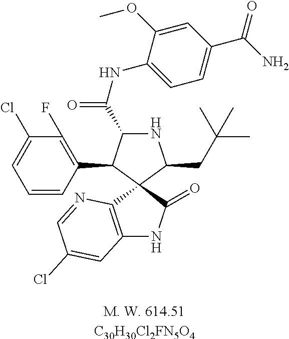

A mixture of rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid (Example 43, 30 mg, 0.050 mmol), N,N-disiopropylethylamine (51.9 mg, 0.402 mmol) and O-(7-azabenzotriazol-1-yl)-N,N,N′,N′-tetramethyluronium hexafluorohposphate (HATU) (Chem-Impex, 30 mg, 0.080 mmol) in DMF (4 mL) was stirred for 20 min before NH₄Cl (7.6 mg, 0.13 mmol) was added. The mixture was stirred for 4 h and poured into EtOAc (120 mL), washed with water (10 mL), sat NH₄Cl (10 mL), sat NaHCO₃ (10 mL), and brine (15 mL) and concentrated. The crude product was purified by RP-HPLC (40% to 100% MeCN in H₂O) to give rac-(2S,3S,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxamide as a white solid (7 mg, 25%). MS (ES⁺) m/z [(M+H)⁺]: 614

### Example 45

### Preparation of intermediate E/Z-5-chloro-3-(3-chloro-2-fluoro-benzylidene)-1,3-dihydro-pyrrolo[3,2-b]pyridin-2-one

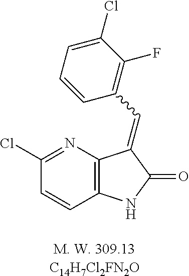

To a suspension of 5-chloro-1H-pyrrolo[3,2-b]pyridin-2(3H)-one (Sinova, 815.5 mg, 4.84 mmol) in MeOH (45 mL) was added 3-chloro-2-fluorobenzaldehyde (Oakwood, 2.3 g, 14.5 mmol). Piperidine (Aldrich, 1.63 g, 19.2 mmol) was added slowly. After stirring a few minutes, the mixture was heated at 50° C. for 6 h resulting in a yellow precipitation. The reaction mixture was cooled and the precipitate was filtered, washed with cold MeOH and dried in vacuum to give E/Z-5-chloro-3-(3-chloro-2-fluoro-benzylidene)-1,3-dihydro-pyrrolo[3,2-b]pyridin-2-one as a light brown solid (1.38 g, 92%). MS (ES⁺) m/z [(M+H)⁺]: 309

### Example 46

### Preparation of methyl rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate

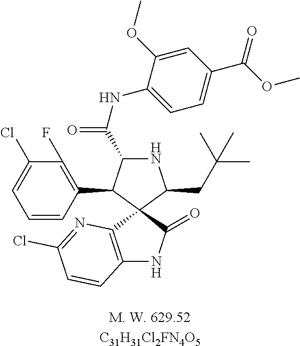

A suspension of E/Z-5-chloro-3-(3-chloro-2-fluoro-benzylidene)-1,3-dihydro-pyrrolo[3,2-b]pyridin-2-one (Example 45, 331.2 mg, 1.07 mmol) in anhydrous THF (25 mL) was warmed to a clear solution and then cooled down to 40 C. LiOH (18 mg, 0.70 mmol) was added and the mixture was stirred at 40° C. for 10 min. (E)-Methyl 4-(2-(3,3-dimethylbutylideneamino)acetamido)-3-methoxybenzoate (Example 3, 360.9 mg, 1.13 mmol) was added in one portion. The reaction mixture was allowed to stir at 40° C. for 20 h, giving a clear reaction mixture. The mixture was diluted with EtOAc (100 mL), washed with water (2×20 mL) and concentrated to a small volume. MeOH was added slowly (˜10 mL) and the mixture was stirred in cold bath for −20 min. The resulting precipitate was filtered, washed with cold MeOH and dried overnight in vacuum to give methyl rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (420 mg, 76%). MS (ES⁺) m/z [(M+H)⁺]: 629

### Example 47

### Preparation of rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid

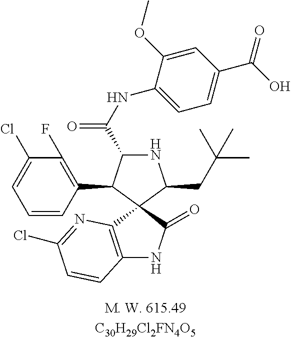

To a suspension of methyl rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (Example 46, 111 mg, 0.17 mmol) in THF (10 mL) was added a solution of LiOH hydrate (61 mg, 1.46 mmol) in water (5 mL). The reaction mixture was stirred at rt overnight before it was treated with 1N HCl to slightly acidic, diluted with ethyl acetate (200 mL), washed with water (15 mL), dried with Na₂SO₄ and concentrated to give rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (107 mg, 98%). MS (ES⁺) m/z [(M+H)⁺]: 615

### Example 48

### Preparation of intermediate E/Z-3-(3-chloro-2-fluoro-benzylidene)-7-methyl-1,3-dihydro-pyrrolo[3,2-b]pyridin-2-one

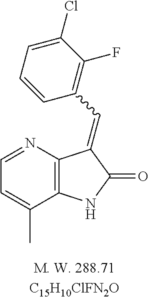

To a suspension of 7-methyl-1H-pyrrolo[3,2-b]pyridin-2(3H)-one (Sinova, 818.0 mg, 5.52 mmol) in MeOH (30 mL) was added 3-chloro-2-fluorobenzaldehyde (Oakwood, 2.60 g, 16.4 mmol) in MeOH (2 mL) to give a clear solution. Piperidine (Aldrich, 1.89 g, 22.2 mmol) was added slowly and a light yellow precipitation started to form shortly. After stirring a few minutes, the reaction mixture was heated at 50° C. for 12 h. The reaction mixture was cooled and the precipitate was filtered, washed with cold MeOH and dried in vacuum to give E/Z-3-(3-chloro-2-fluoro-benzylidene)-7-methyl-1,3-dihydro-pyrrolo[3,2-b]pyridin-2-one as a light brown solid (939 mg, 58%). MS (ES⁺) m/z [(M+H)⁺]: 288

### Example 49

### Preparation of methyl rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoate

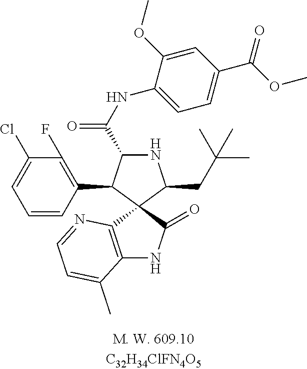

To a suspension of E/Z-3-(3-chloro-2-fluoro-benzylidene)-7-methyl-1,3-dihydro-pyrrolo[3,2-b]pyridin-2-one (Example 48, 318 mg, 1.10 mmol) in anhydrous THF (25 mL) was added LiOH (20 mg, 0.85 mmol) and the mixture was stirred at 40° C. for 10 min. (E)-Methyl 4-(2-(3,3-dimethylbutylideneamino)acetamido)-3-methoxybenzoate (Example 3, 371 mg, 1.16 mmol) was added in one portion. The reaction mixture was allowed to stir at 40° C. for 20 h. The mixture was diluted with EtOAc (100 mL) and washed with water, brine and dried over Na₂SO₄ and concentrated. The crude product was purified on flash chromatography (EtOAc/CH₂Cl₂, 3/97 to 20/20) to give methyl rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (171 mg, 26%). MS (ES⁺) m/z [(M+H)⁺]: 609

### Example 50

### Preparation of rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid

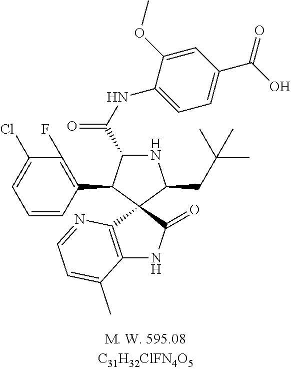

To a suspension of methyl rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoate (Example 49, 150 mg, 0.246 mmol) in THF (14 mL) was added LiOH monohydrate (83 mg, 1.98 mmol) in water (5 mL). The mixture was stirred at rt overnight. The mixture was then treated with 1N HCl to slightly acidic, diluted with ethyl acetate (100 mL), washed with water (10 mL), dried with Na₂SO₄ and concentrated to give rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (110 mg, 75%). MS (ES⁺) m/z [(M+H)⁺]: 595

### Example 51

### Preparation of chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-e]pyridine]-5-ylcarboxamido)-3-methoxybenzoate(A)

### and

### chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate(B)

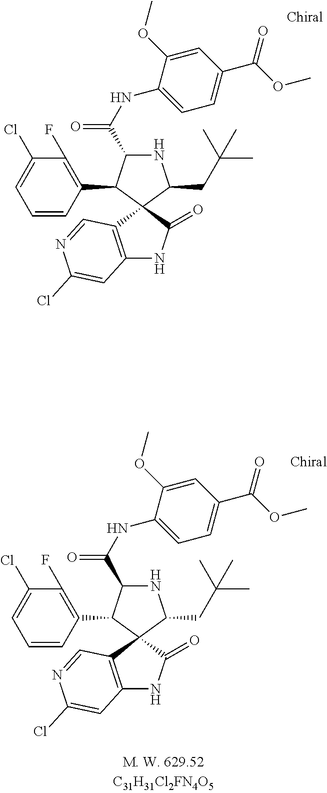

Methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (Example 24) was separated by SFC Chromatography (Waters/Thar Multi-Gram II, Kromasil 5-CelluCoat OD 3×25 cm., 35° C. at 100 bar, eluting with 40% ethanol in carbon dioxide) to give chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate, MS (ES⁺) m/z [(M+H)⁺]: 629, and chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate. MS (ES⁺) m/z [(M+H)⁺]: 629

### Example 52

### Preparation of chiral 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid

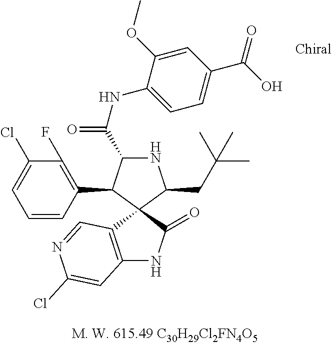

To a solution of chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (Example 51, 56 mg, 0.090 mmol) in THF (6 mL) was added a solution of LiOH hydrate (31 mg, 0.74 mmol) in water (3 mL). The reaction mixture was stirred at rt overnight before it was treated with 1N HCl to slightly acidic. The mixture was partitioned between EtOAc (100 mL) and water (10 mL), washed with water, brine and dried over Na₂SO₄, concentrated and lyophized to give chiral 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white powder (46 mg, 84%). MS (ES⁺) m/z [(M+H)⁺]: 615

### Example 53

### Preparation of chiral(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

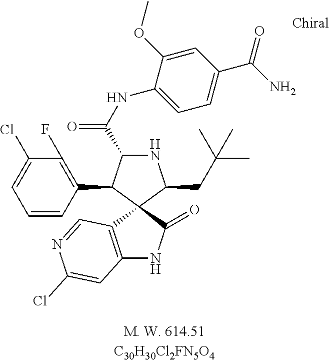

A mixture of chiral 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid (Example 52, 26 mg, 0.043 mmol) and 1,1′-carbonyldiimidazole (Aldrich, 26 mg, 0.16 mmol) in THF (3 mL) was stirred at rt for 17 hrs. Ammonium hydroxide (180 mg, 5.14 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 3/97) to give chiral(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (21 mg, 79%). MS (ES⁺) m/z [(M+H)⁺]: 614

### Example 54

### Preparation of chiral 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid

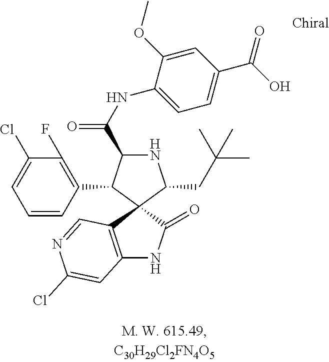

To a solution of chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate (62 mg, 0.099 mmol, Example 51) in THF (6 mL) was added a solution of LiOH hydrate (34 mg, 0.82 mmol) in water (3 mL). The reaction mixture was stirred at rt overnight before it was treated with 1N HCl to slightly acidic. The mixture was partitioned between EtOAc (100 mL) and water (10 mL), washed with water, brine and dried over Na₂SO₄, concentrated and lyophized to give chiral 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid as a white powder (50 mg, 81%). MS (ES⁺) m/z [(M+H)⁺]: 615

### Example 55

### Preparation of chiral(2R,3S,4R,5S)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide

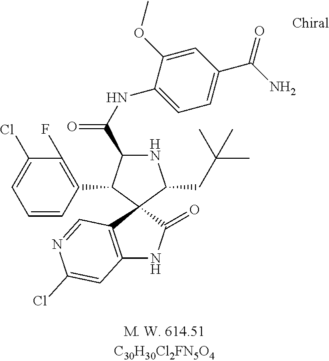

A mixture of chiral 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid (Example 54, 29 mg, 0.047 mmol) and 1,1′-carbonyldiimidazole (Aldrich, 23 mg, 0.14 mmol) in THF (3 mL) was stirred at rt for 17 h. Ammonium hydroxide (180 mg, 5.14 mmol) was added and the mixture was stirred for 20 min. The mixture was partitioned between EtOAc (75 mL) and water (10 mL), washed with sat. NaHCO₃ (10 mL), water (10 mL) then sat. NH₄Cl (10 mL). The organic layer was dried over Na₂SO₄ and concentrated in vacuo onto silica gel. The crude material was purified by flash chromatography (ethanol/CH₂Cl₂, 0.5/99.5 to 3/97) to give chiral (2R,3S,4R,5S)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide as a white solid (20 mg, 70%). MS (ES⁺) m/z [(M+H)⁺]: 614

### Example 56

### Preparation of chiral 4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid(A)

### and chiral 4-((2R,3R,4R,5S)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid(B)

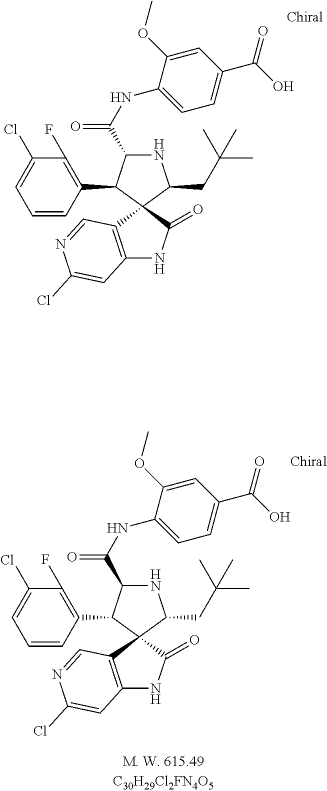

A rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid (Example 43) was separated by SFC Chromatography SFC (Waters/Thar Multi-Gram II, Chiral Technologies, Diacel OD, 3×25 cm., 35° C. at 100 bar, eluting with 35% Ethanol in carbon dioxide) to give chiral 4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid, MS (ES⁺) m/z [(M+H)⁺]: 615, and chiral 4-((2R,3R,4R,5S)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid. MS (ES⁺) m/z [(M+H)⁺]: 615

### Example 57

### Preparation of chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate(A)

### and chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate(B)

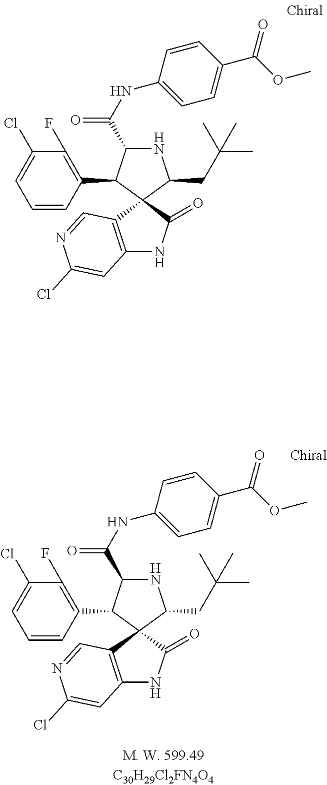

Rac-methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate (Example 27) was separated by SFC (Waters/Thar Multi-Gram II, Chiral Technologies, Diacel IA, 3×25 cm., 35° C. at 100 bar, eluting with 35% ethanol in carbon dioxide) to give chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate, MS (ES⁺) m/z [(M+H)⁺]: 599, and chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate. MS (ES⁺) m/z [(M+H)⁺]: 599

### Example 58

### Preparation of intermediate E/Z-6-chloro-3-(3-chloro-benzylidene)-1,3-dihydro-pyrrolo[3,2-b]pyridin-2-one

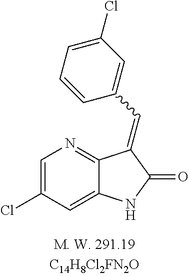

To the mixture of 6-chloro-4-aza-2-oxindole (Sinova, 1 g, 5.9 mmol) and 3-chloro-benzaldehyde (Aldrich, 0.83 g, 5.9 mmol) in methanol (50 mL) was added piperidine (Aldrich, 0.5 g, 5.9 mmol) dropwise. The reaction mixture was heated at 80° C. and stirred for 1 h. Then the mixture was cooled to room temperature and filtered. The resulting precipitate was collected and dried to give the first batch of desired product. The filtrate was concentrated, and the residue was purified by chromatography (0-25% EtOAc in dichlormethane) to give the second batch of product. The two batches were combined to give E/Z-6-chloro-3-(3-chloro-benzylidene)-1,3-dihydro-pyrrolo[3,2-b]pyridin-2-one as an orange solid (0.9 g, 52%).

### Example 59

### Preparation of methyl rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoate

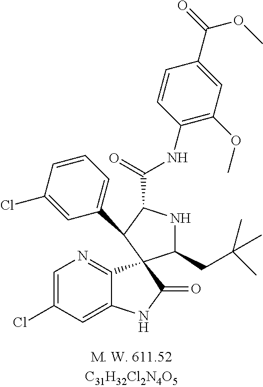

To a solution of E/Z-6-chloro-3-(3-chloro-benzylidene)-1,3-dihydro-pyrrolo[3,2-b]pyridin-2-one (Example 58, 0.25 g, 0.86 mmol) in tetrahydrofuran (2 mL) was added anhydrous LiOH (41 mg, 1.72 mmol). The mixture was warmed to 40° C. and stirred for 10 min. 4-{2-[3,3-dimethyl-but-(E)-ylideneamino]-acetylamino}-3-methoxy-benzoic acid methyl ester

(Example 3, 0.27 g, 0.86 mmol) was added in one portion. The reaction mixture was stirred at 40° C. for 2 h. The mixture was cooled to room temperature and filtered. The resulting precipitate was collected, washed with ethyl acetate, and dried to give methyl rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoate as an off-white solid (0.15 g, 29%). MS (ES⁺) m/z Calcd for C₃₁H₃₂Cl₂N₄O₅+H [(M+H)⁺]: 611. found: 611.

### Example 60

### Preparation of rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoic acid

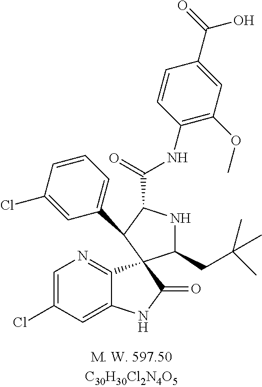

To a solution of methyl rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoate (Example 59, 70 mg, 0.11 mmol) in tetrahydrofuran (9 mL) was added an aqueous solution (1 N) of NaOH (1.1 mL, 1.1 mmol). The reaction mixture was stirred at room temperature for 5 h. The “pH” of the mixture was adjusted to 5 by aqueous HCl solution, then concentrated to a small volume. The residue was partitioned between ethyl acetate and water. The organic layer was separated, and the aqueous layer was extracted with ethyl acetate twice. The organic extracts were combined, washed with water, brine, dried over MgSO₄, and concentrated to give rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoic acid as an off-white solid (58 mg, 85%). MS (ES⁺) m/z Calcd for C₃₀H₃₀Cl₂N₄O₅+H [(M+H)⁺]: 597. found: 597.

### Example 61

### Preparation of intermediate N-(4-cyano-2-methoxy-phenyl)-2-[3,3-dimethyl-but-(E)-ylideneamino]-acetamide

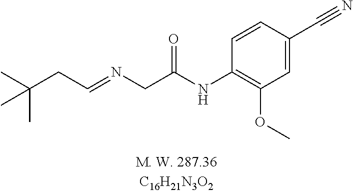

Step a: A mixture of 3-methoxy-4-nitrobenzoic acid (Acros, 10 g, 51 mmol) in thionyl chloride (36 g) was heated at reflux for 2 h. The mixture was concentrated. To the residue was added a methanolic solution (7 N) of ammonia. The reaction mixture was stirred at room temperature for 72 h. The mixture was concentrated, and the residue was partitioned between ethyl acetate and water. The precipitate between the two layers was filtered and collected to give 3-methoxy-4-nitrobenzamide as a light yellow solid (8 g, 81%).

Step b: To a solution of 3-methoxy-4-nitrobenzamide (8 g, 41 mmol) in dioxane (300 mL) was added pyridine (32 g, 408 mmol), followed by dropwise addition of trifluoroacetic anhydride (43 g, 204 mmol). The reaction mixture was stirred at room temperature for 5 h. Water was added to quench the reaction. The mixture was concentrated, then the residue was partitioned between ethyl acetate and water. The organic layer was separated, the aqueous layer was extracted with ethyl acetate. The organic layers were combined, washed with water, aqueous saturated CuSO₄ solution, brine, dried over MgSO₄, and concentrated to give 3-methoxy-4-nitrobenzonitrile as a off white solid (6.5 g, 90%)

Step c: To the suspension of 3-methoxy-4-nitrobenzonitrile (11.4 g, 64 mmol) in ethyl acetate (60 mL) was added 10% Pd/C (1 g). The reaction mixture was vigorously shaken in a Parr under an atmosphere of hydrogen (50 psi) at room temperature for 45 min. The mixture was filtered through a short pad of celite, and the filtrate was concentrated to give 4-amino-3-methoxy-benzonitrile as a yellow oil, which solidified at stand (9.5 g, 95%)

Step d: To a solution of 2-(tert-butoxycarbonylamino)acetic acid (Advanced Chemical, 3.9 g, 22.3 mmol) and N1-((ethylimino)methylene)-N3,N3-dimethylpropane-1,3-diamine hydrochloride (EDCI) (Aldrich, 4.27 g, 22.3 mmol) in dichloromethane (20 mL) was added 4-amino-3-methoxy-benzonitrile (2 g, 13.5 mmol). The reaction mixture was stirred at room temperature for 20 h. The mixture was concentrated, and the residue was partitioned between ethyl acetate and saturated aqueous NH₄Cl solution. The organic layer was separated, and aqueous layer was extracted with ethyl acetate twice. The combined organic extract was washed with saturated aqueous NaHCO₃ solution, brine, dried over MgSO₄, and concentrated. The residue was purified by chromatography (0-15% EtOAc in dichlormethane) to give tert-butyl 2-(4-cyano-2-methoxyphenylamino)-2-oxoethylcarbamate as a white solid (3.3 g, 80%).

Step e: To a solution of tert-butyl 2-(4-cyano-2-methoxyphenylamino)-2-oxoethylcarbamate (1.5 g, 4.9 mmol) in dichloromethane (10 mL) was added trifluoroacetic acid (10 mL). The reaction mixture was stirred at room temperature for 1 h, then concentrated. The residue was then triturated with hexanes, concentrated, dried in vacuo to give 2-amino-N-(4-cyano-2-methoxyphenyl)acetamide trifluoroacetic acid as a yellow solid (1.2 g, 77%).

Step f: To a mixture of 2-amino-N-(4-cyano-2-methoxyphenyl)acetamide trifluoroacetic acid (1.7 g, 5.4 mmol) in methyl tert-butyl ether (20 mL) was added triethylamine (0.78 mL, 5.7 mmol). The mixture was stirred at room temperature for 30 min. Then 3,3-dimethyl-butyraldehyde (Aldrich, 0.57 g, 5.7 mmol) was added. The reaction mixture was stirred at room temperature for 3.5 h. The mixture was filtered, and the filtrate was concentrated. The residue was partitioned between dichloromethane and water. The organic layer was separated, and aqueous layer was extracted with dichlormethane. The combined organic extract was washed with water, dried over MgSO₄, and concentrated to give N-(4-cyano-2-methoxy-phenyl)-2-[3,3-dimethyl-but-(E)-ylideneamino]-acetamide (1.2 g, 77%) as a yellow foam which was used in the next step without further purification.

### Example 62

### Preparation of rac-(2S,3S,4R,5R)-6′-chloro-4-(3-chlorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide

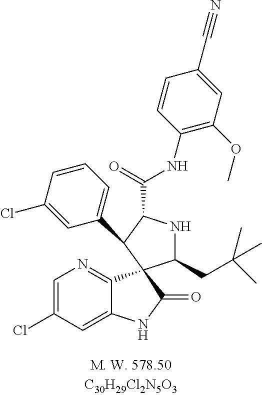

To a solution of E/Z-6-chloro-3-(3-chloro-benzylidene)-1,3-dihydro-pyrrolo[3,2-b]pyridin-2-one (Example 58, 0.1 g, 0.34 mmol) in tetrahydrofuran (2 mL) was added anhydrous LiOH (16.5 mg, 0.69 mmol). The mixture was warmed to 40° C. and stirred for 10 min. N-(4-cyano-2-methoxy-phenyl)-2-[3,3-dimethyl-but-(E)-ylideneamino]-acetamide (Example 61, 0.15 g, 0.52 mmol) was added in one portion. The reaction mixture was stirred at 40° C. for 2 h. The mixture was cooled to room temperature and filtered. The resulting precipitate was collected, washed with ethyl acetate, and dried to give rac-(2S,3S,4R,5R)-6′-chloro-4-(3-chlorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide as a white solid (33 mg, 17%). MS (ES⁺) m/z Calcd for C₃₀H₂₉Cl₂N₅O₃+H [(M+H)⁺]: 578. found: 578.

### Example 63

### Preparation of rac-(2S,3S,4R,5R)-N-(4-carbamoyl-2-methoxyphenyl)-6′-chloro-4-(3-chlorophenyl)-2-neopentyl-2′-oxo-1′,2′-dihydrospiro[pyrrolidine-3,3′-pyrrolo[3,2-b]pyridine]-5-carboxamide

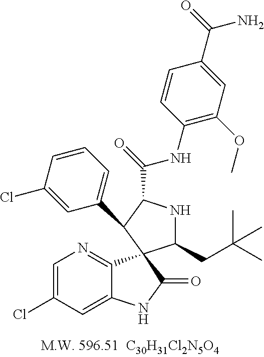

To the solution of rac-(2S,3S,4R,5R)-6′-chloro-4-(3-chlorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide (Example 62, 26 mg, 0.045 mmol) in DMSO (0.2 mL) at 0° C. was added an aqueous solution (30%) of H₂O₂ (Aldrich, 0.076 mg, 0.67 mmol), followed by the addition of aqueous solution (1 N) of NaOH (0.23 mL, 0.23 mmol). The reaction mixture was stirred at 0° C. for 1 h. The mixture was partitioned between ethyl acetate and saturated aqueous Na₂SO₃ solution. The organic layer was separated, washed with water, brine, dried over MgSO₄, and concentrated to give rac-(2S,3S,4R,5R)-N-(4-carbamoyl-2-methoxyphenyl)-6′-chloro-4-(3-chlorophenyl)-2-neopentyl-2′-oxo-1′,2′-dihydrospiro[pyrrolidine-3,3′-pyrrolo[3,2-b]pyridine]-5-carboxamide as an off-white solid (24 mg, 90%). MS (ES⁺) m/z Calcd for C₃₀H₃₁Cl₂N₅O₄+H [(M+H)⁺]: 596. found: 596.

### Example 64

### Preparation of intermediate E/Z-3-(3-bromo-2-fluoro-benzylidene)-6-chloro-1,3-dihydro-pyrrolo[3,2-b]pyridin-2-one

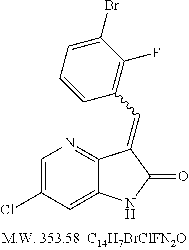

To the mixture of 6-chloro-4-aza-2-oxindole (Sinova, 1 g, 5.9 mmol) and 3-bromo-2-fluorobenzaldehyde (Aldrich, 1.6 g, 7.9 mmol) in methanol (30 mL) was added piperidine (Aldrich, 2 g, 24 mmol) dropwise. The reaction mixture was heated at 50° C. and stirred for 3 h. Then the mixture was cooled to room temperature and filtered. The resulting precipitate was collected and dried to give the first batch of desired product. The filtrate was concentrated, and the residue was purified by chromatography (25-50% EtOAc in hexanes) to give the second batch of product. The two batches were combined to give E/Z-3-(3-bromo-2-fluoro-benzylidene)-6-chloro-1,3-dihydro-pyrrolo[3,2-b]pyridin-2-one as a yellow solid (1.6 g, 76%).

### Example 65

### Preparation of methyl rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate

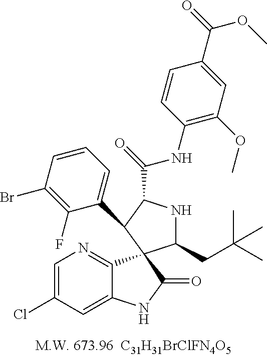

To a solution of E/Z-3-(3-bromo-2-fluoro-benzylidene)-6-chloro-1,3-dihydro-pyrrolo[3,2-b]pyridin-2-one (Example 64, 0.3 g, 0.85 mmol) in tetrahydrofuran (10 mL) was added anhydrous LiOH (41 mg, 1.7 mmol). The mixture was warmed to 40° C. and stirred for 10 min. 4-{2-[3,3-dimethyl-but-(E)-ylideneamino]-acetylamino}-3-methoxy-benzoic acid methyl ester (Example 3, 0.27 g, 0.85 mmol) was added in one portion. The reaction mixture was stirred at 40° C. for 18 h. The mixture was cooled to room temperature and filtered. The resulting precipitate was collected, washed with ethyl acetate, and dried to give the first batch of desired product. The filtrate was concentrated, and the residue was purified by chromatography (5-10% EtOAc in dichlormethane) to give the second batch of desired product. The two batches were combined to give methyl rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate as a white solid (0.17 g, 30%). MS (ES⁺) m/z Calcd for C₃₁H₃₁BrClFN₄O₅+H [(M+H)⁺]:673. found: 673.

### Example 66

### Preparation of methyl rac-4-{(2S,3R,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate

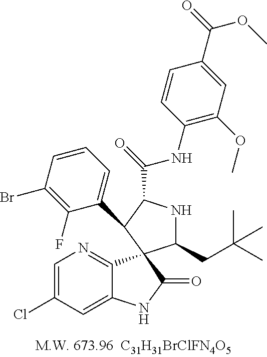

In the preparation of rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate as described in Example 65, methyl rac-4-{(2S,3R,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate was obtained as another product by chromatography (5-10% EtOAc in dichlormethane): 50 mg, 9%, off white solid. MS (ES⁺) m/z Calcd for C₃₁H₃₁BrClFN₄O₅+H [(M+H)⁺]:673. found: 673.

### Example 67

### Preparation of rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoic acid

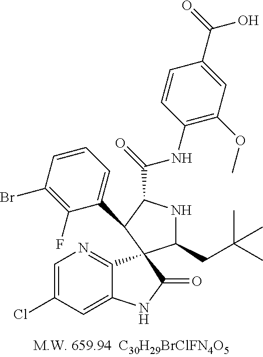

To a solution of methyl rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate (Example 65, 120 mg, 0.18 mmol) in tetrahydrofuran (8 mL) was added an aqueous solution (1 N) of NaOH (1 mL, 1 mmol). The reaction mixture was stirred at room temperature for 18 h. The “pH” of the mixture was adjusted to 3 by aqueous HCl solution, then concentrated to a small volume. The residue was partitioned between ethyl acetate and water. The organic layer was separated, and the aqueous layer was extracted with ethyl acetate twice. The organic extracts were combined, washed with water, brine, dried over MgSO₄, and concentrated to give rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoic acid as a white solid (80 mg, 68%). MS (ES⁺) m/z Calcd for C₃₀H₂₉BrClFN₄O₅+H [(M+H)⁺]: 659. found: 659.

### Example 68

### Preparation of rac-(2S,3S,4S,5R)-4-(3-bromo-2-fluorophenyl)-6′-chloro-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide

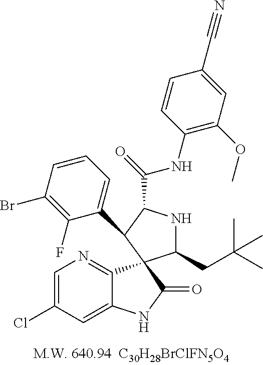

To a solution of E/Z-3-(3-bromo-2-fluoro-benzylidene)-6-chloro-1,3-dihydro-pyrrolo[3,2-b]pyridin-2-one (Example 64, 0.1 g, 0.28 mmol) in tetrahydrofuran (1 mL) was added anhydrous LiOH (13.5 mg, 0.57 mmol). The mixture was warmed to 40° C. and stirred for 10 min. N-(4-cyano-2-methoxy-phenyl)-2-[3,3-dimethyl-but-(E)-ylideneamino]-acetamide (Example 61, 0.12 g, 0.42 mmol) was added in one portion. The reaction mixture was stirred at 40° C. for 1 h. The mixture was cooled to room temperature and filtered. The resulting precipitate was collected, washed with ethyl acetate, and dried to give rac-(2S,3S,4S,5R)-4-(3-bromo-2-fluorophenyl)-6′-chloro-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide as a white solid (40 mg, 22%). MS (ES⁺) m/z Calcd for C₃₀H₂₈BrClFN₅O₄+H [(M+H)⁺]: 640. found: 640.

### Example 69

### Preparation of rac-(2S,3S,4S,5R)-4-(3-bromo-2-fluorophenyl)-N-(4-carbamoyl-2-methoxyphenyl)-6′-chloro-2-neopentyl-2′-oxo-1′,2′-dihydrospiro[pyrrolidine-3,3′-pyrrolo[3,2b]pyridine]-5-carboxamide

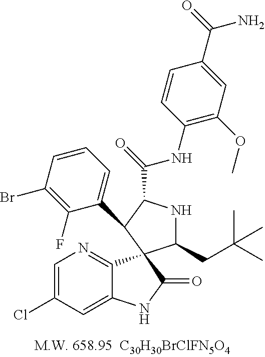

To a solution of rac-(2S,3S,4S,5R)-4-(3-bromo-2-fluorophenyl)-6′-chloro-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide (Example 68, 32 mg, 0.05 mmol) in DMSO (0.2 mL) at 0° C. was added an aqueous solution (30%) of H₂O₂ (Aldrich, 0.085 mg, 0.75 mmol), followed by the addition of aqueous solution (1 N) of NaOH (0.25 mL, 0.25 mmol). The reaction mixture was stirred at 0° C. for 1 h. The mixture was partitioned between ethyl acetate and saturated aqueous Na₂SO₃ solution. The organic layer was separated, washed with water, brine, dried over MgSO₄, and concentrated to give rac-(2S,3S,4S,5R)-4-(3-bromo-2-fluorophenyl)-N-(4-carbamoyl-2-methoxyphenyl)-6′-chloro-2-neopentyl-2′-oxo-1′,2′-dihydrospiro[pyrrolidine-3,3′-pyrrolo[3,2-b]pyridine]-5-carboxamide as a off white solid (29 mg, 88%). MS (ES⁺) m/z Calcd for C₃₀H₃₀BrClFN₅O₄+H [(M+H)⁺]: 658. found: 658.

### Example 70

### Preparation of intermediate acetic acid 2-(4-{2-[3,3-dimethyl-but-(E)-ylideneamino]-acetylamino}-3-methoxy-phenoxy)-ethyl ester

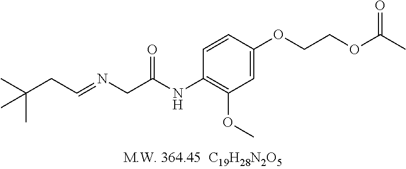

Step a: To a solution of 4-fluoro-2-methoxy-1-nitrobenzene (Combi-blocks, 3.4 g, 19.9 mmol) in DMSO (40 mL) was added an aqueous solution (1 N) of NaOH (40 mL, 40 mmol). The reaction mixture was heated at 80° C. for 20 h. The mixture was cooled to room temperature, and the “pH” of the solution was adjusted to 5 by aqueous HCl solution. The mixture was extracted with ethyl acetate three times. The combined organic extract was washed with water, brine, dried over MgSO₄, and concentrated to give 3-methoxy-4-nitrophenol as a light yellow solid (3.2 g, 95%).

Step b: To a solution of 3-methoxy-4-nitrophenol (1 g, 5.9 mmol) in anhydrous DMF (25 mL) were added K₂CO₃ (2.45 g, 17.7 mmol) and (2-bromoethoxy)(tert-butyl)dimethylsilane (1.7 g, 7.1 mmol) sequentially. The reaction mixture was heated at 70° C. for 20 h. The mixture was cooled to room temperature, and diluted with water. The mixture was extracted with ethyl acetate three times. The combined organic extract was washed with water, brine, dried over MgSO₄, and concentrated. The residue was purified by chromatography (0-20% EtOAc in hexanes) to give tert-butyl-[2-(3-methoxy-4-nitro-phenoxy)-ethoxy]-dimethyl-silane as a light yellow oil (1.0 g, 52%).

Step c: To a solution of tert-butyl-[2-(3-methoxy-4-nitro-phenoxy)-ethoxy]-dimethyl-silane (4 g, 12.2 mmol) in THF (50 mL) was added an aqueous HCl solution (1 N, 20 mL, 20 mmol). The reaction mixture was stirred at room temperature for 1 h. The mixture was concentrated. The residue was partitioned between ethyl acetate and saturated aqueous NaHCO₃ solution. The organic layer was separated, and aqueous layer was extracted with ethyl acetate. The combined organic extract was washed with water, brine, dried over MgSO₄, and concentrated to give 2-(3-methoxy-4-nitrophenoxy)ethanol as an off white solid (2.1 g, 81%).

Step d: To a solution of 2-(3-methoxy-4-nitrophenoxy)ethanol (2.1 g, 9.9 mmol) and pyridine (0.9 g, 11.4 mmol) in THF (50 mL) at 0° C. was acetyl chloride (0.89 g, 11.4 mmol). The reaction mixture was warmed to room temperature and stirred for 1 h. The mixture was concentrated. The residue was partitioned between ethyl acetate and water. The organic layer was separated, and aqueous layer was extracted with ethyl acetate. The combined organic extract was washed with water, saturated aqueous CuSO₄ solution, brine, dried over MgSO₄, and concentrated. The residue was purified by chromatography (0-40% EtOAc in dichloromethane) to give 2-(3-methoxy-4-nitrophenoxy)ethyl acetate as a yellow foam (2.4 g, 95%).

Step e: A suspension of 2-(3-methoxy-4-nitrophenoxy)ethyl acetate (2.4 g, 9.4 mmol) and Pd/C (Aldrich, 10%, 0.4 g) in ethyl acetate (30 mL) was vigorously shaken in a Parr under atmosphere of H₂ (50 psi) for 0.5 h. The mixture was filtered through a short pad of celite. The filtrate was concentrated to give acetic acid 2-(4-amino-3-methoxy-phenoxy)-ethyl ester as a light brown oil (2 g, 94%).

Step f: To a solution of 2-(tert-butoxycarbonylamino)acetic acid (Advanced Chemical, 2.57 g, 14.7 mmol) and EDCI (Aldrich, 2.81 g, 14.7 mmol) in dichloromethane (20 mL) was added 2-(4-amino-3-methoxy-phenoxy)-ethyl ester (2 g, 8.9 mmol). The reaction mixture was stirred at room temperature for 20 h. The mixture was concentrated, and the residue was partitioned between dichloromethane and saturated aqueous NH₄Cl solution. The organic layer was separated, and aqueous layer was extracted with dichloromethane twice. The combined organic extract was washed with saturated aqueous NaHCO₃ solution, brine, dried over MgSO₄, and concentrated to give 2-(4-(2-(tert-butoxycarbonylamino)acetamido)-3-methoxyphenoxy)ethyl acetate as a light brown oil (3.3 g, 97%).

Step g: A solution of 2-(4-(2-(tert-butoxycarbonylamino)acetamido)-3-methoxyphenoxy)ethyl acetate (1 g, 2.6 mmol) in dichloromethane (10 mL) was added trifluoroacetic acid (10 mL). The reaction mixture was stirred at room temperature for 1 h, then concentrated. The residue was then triturated with hexanes, concentrated, dried in vacuo to give 2-(4-(2-aminoacetamido)-3-methoxyphenoxy)ethyl acetate trifluoroacetic acid as an off white foam (0.8 g, 77%).

Step h: To a mixture of 2-(4-(2-aminoacetamido)-3-methoxyphenoxy)ethyl acetate trifluoroacetic acid (1 g, 2.5 mmol) in methyl tert-butyl ether (12 mL) was added triethylamine (0.53 mL, 3.8 mmol). The mixture was stirred at room temperature for 30 min. Then 3,3-dimethyl-butyraldehyde (Aldrich, 0.33 g, 2.7 mmol) was added. The reaction mixture was stirred at room temperature for 20 h. The mixture was filtered, and the filtrate was concentrated. The residue was partitioned between dichloromethane and water. The organic layer was separated, and aqueous layer was extracted with dichlormethane. The combined organic extract was washed with water, dried over MgSO₄, and concentrated to give acetic acid 2-(4-{2-[3,3-dimethyl-but-(E)-ylideneamino]-acetylamino}-3-methoxy-phenoxy)-ethyl ester as a yellow oil (0.9 g, 98%) which was used in the next step without further purification.

### Example 71

### Preparation of rac-2-(4-((2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido)-3-methoxyphenoxy)ethyl acetate

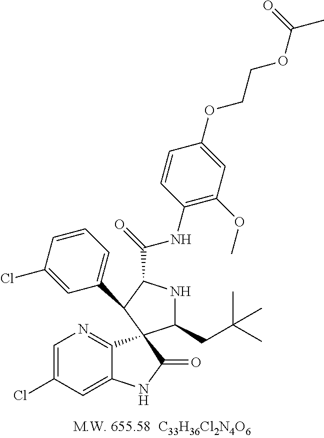

To a solution of E/Z-6-chloro-3-(3-chloro-benzylidene)-1,3-dihydro-pyrrolo[3,2-b]pyridin-2-one (Example 58, 0.14 g, 0.48 mmol) in tetrahydrofuran (3 mL) was added anhydrous LiOH (23 mg, 0.96 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.18 g, 0.48 mmol) was added in one portion. The reaction mixture was stirred at 40° C. for 2 h. The mixture was cooled to room temperature and filtered through a short pad of silica gel. The silica gel was washed with ethyl acetate. The filtrate was concentrated, and the residue was purified by chromatography (0-20% EtOAc in dichloromethane) to give rac-2-(4-((2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido)-3-methoxyphenoxy)ethyl acetate as an off white solid (0.1 g, 32%).

### Example 72

### Preparation of rac-(2S,3S,4R,5R)-6′-chloro-4-(3-chlorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide

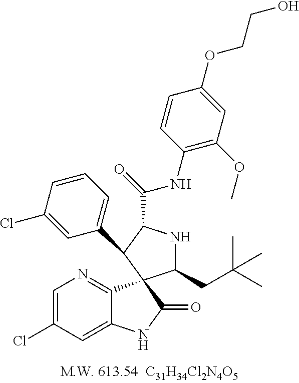

To a solution of rac-2-(4-((2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido)-3-methoxyphenoxy)ethyl acetate (Example 71, 0.1 g, 0.15 mmol) in tetrahydrofuran (2 mL) was added an aqueous solution (1 N) of NaOH (2 mL, 2 mmol). The reaction mixture was stirred at room temperature for 3 h. The mixture was concentrated. The residue was partitioned between ethyl acetate and saturated aqueous NaHCO₃ solution. The organic layer was separated, and the aqueous layer was extracted with ethyl acetate twice. The organic extracts were combined, washed with water, brine, dried over MgSO₄, and concentrated. The residue was recrystallized in methanol to give rac-(2S,3S,4R,5R)-6′-chloro-4-(3-chlorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as a white solid (11 mg, 12%). MS (ES⁺) m/z Calcd for C₃₁H₃₄Cl₂N₄O₅+H [(M+H)⁺]: 613. found: 613.

### Example 73

### Preparation of rac-(2S,3S,4S,5R)-4-(3-bromo-2-fluorophenyl)-6′-chloro-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide

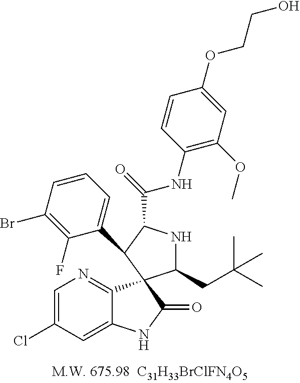

To a solution of E/Z-3-(3-bromo-2-fluoro-benzylidene)-6-chloro-1,3-dihydro-pyrrolo[3,2-b]pyridin-2-one (Example 64, 0.1 g, 0.28 mmol) in tetrahydrofuran (1 mL) was added anhydrous LiOH (14 mg, 0.57 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.16 g, 0.42 mmol) was added in one portion. The reaction mixture was stirred at 40° C. for 3 h. Then an aqueous solution (1 N) of NaOH (1 mL, 1 mmol) was added, and the reaction mixture was stirred at 40° C. for 1 h. The mixture was poured into water, and extracted with ethyl acetate three time. The combined extract was washed with water, brine, dried over MgSO₄, and concentrated. The residue was purified by chromatography (0-100% EtOAc in dichloromethane) to give rac-(2S,3S,4S,5R)-4-(3-bromo-2-fluorophenyl)-6′-chloro-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as a light yellow solid (40 mg, 21%).

MS (ES⁺) m/z Calcd for C₃₁H₃₃BrClFN₄O₅+H [(M+H)⁺]: 675. found: 675.

### Example 74

### Preparation of rac-(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide

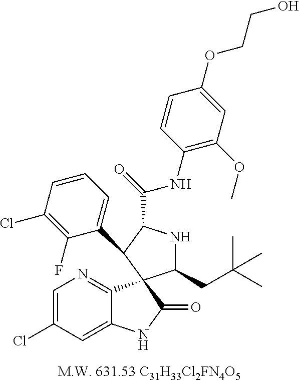

To a solution of E/Z-6-chloro-3-(3-chloro-2-fluoro-benzylidene)-1,3-dihydro-pyrrolo[3,2-b]pyridin-2-one (Example 40, 0.15 g, 0.49 mmol) in tetrahydrofuran (1.5 mL) was added anhydrous LiOH (23 mg, 0.97 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.27 g, 0.73 mmol) was added in one portion. The reaction mixture was stirred at 40° C. for 2 h. Then an aqueous solution (1 N) of NaOH (1 mL, 1 mmol) was added, and the reaction mixture was stirred at 40° C. for 1 h. The mixture was poured into water, and extracted with ethyl acetate three time. The combined extract was washed with water, brine, dried over MgSO₄, and concentrated. The residue was purified by chromatography (0-100% EtOAc in dichloromethane) to give rac-(2S,3S,4S,5R)-4-(3-bromo-2-fluorophenyl)-6′-chloro-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as a yellow solid (51 mg, 17%). MS (ES⁺) m/z Calcd for C₃₁H₃₃Cl₂FN₄O₅+H [(M+H)⁺]: 631. found: 631.

### Example 75

### Preparation of chiral(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide

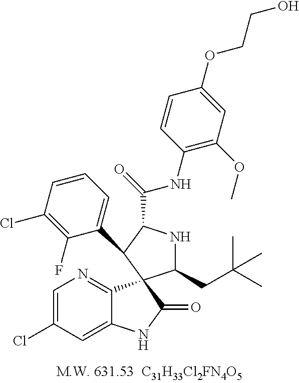

Rac-(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide (Example 74, 0.11 g) was separated by chiral SFC chromatography to provide chiral(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as a off white solid (10 mg, 9%) and chiral(2R,3R,4R,5S)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as an off white solid (12 mg, 11%).

MS (ES⁺) m/z Calcd for C₃₁H₃₃Cl₂FN₄O₅+H [(M+H)⁺]: 631. found: 631.

### Example 76

### Preparation of intermediate E/Z-6-bromo-3-(3-chloro-2-fluoro-benzylidene)-1,3-dihydro-pyrrolo[3,2-b]pyridin-2-one

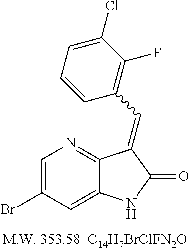

To the mixture of 6-bromo-4-aza-2-oxindole (Sinova, 0.3 g, 1.4 mmol) and 3-chloro-2-fluorobenzaldehyde (Oakwood, 0.45 g, 2.8 mmol) in methanol (20 mL) was added piperidine (Aldrich, 0.36 g, 4.2 mmol) dropwise. The reaction mixture was heated at 50° C. and stirred for 3 h. Then the mixture was cooled to room temperature and filtered. The resulting precipitate was collected and dried to give the first batch of desired product. The filtrate was concentrated, and the residue was purified by chromatography (25-50% EtOAc in hexanes) to give the second batch of product. The two batches were combined to give E/Z-6-bromo-3-(3-chloro-2-fluoro-benzylidene)-1,3-dihydro-pyrrolo[3,2-b]pyridin-2-one as a yellow solid (0.35 g, 70%).

### Example 77

### Preparation of methyl rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate

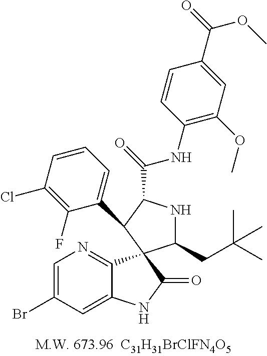

To a solution of E/Z-6-bromo-3-(3-chloro-2-fluoro-benzylidene)-1,3-dihydro-pyrrolo[3,2-b]pyridin-2-one (Example 76, 0.16 g, 0.45 mmol) in tetrahydrofuran (10 mL) was added anhydrous LiOH (11 mg, 0.45 mmol). The mixture was warmed to 40° C. and stirred for 10 min. 4-{2-[3,3-dimethyl-but-(E)-ylideneamino]-acetylamino}-3-methoxy-benzoic acid methyl ester (Example 3, 0.15 g, 0.48 mmol) was added in one portion. The reaction mixture was stirred at 40° C. for 18 h. The mixture was cooled to room temperature and filtered. The resulting precipitate was collected, washed with ethyl acetate, and dried to give the first batch of desired product. The filtrate was concentrated, and the residue was purified by chromatography (5-10% EtOAc in dichlormethane) to give the second batch of desired product. The two batches were combined to give methyl rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (32 mg, 10%).

MS (ES⁺) m/z Calcd for C₃₁H₃₁BrClFN₄O₅+H [(M+H)⁺]: 673. found: 673.

### Example 78

### Preparation of rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid

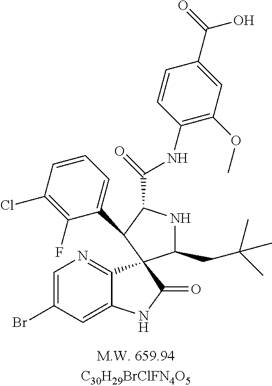

To a solution of methyl rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (Example 77, 25 mg, 0.037 mmol) in tetrahydrofuran (6 mL) was added an aqueous solution (1 N) of NaOH (2 mL, 2 mmol). The reaction mixture was stirred at room temperature for 20 h. The “pH” of the mixture was adjusted to 3 by aqueous HCl solution, then concentrated to a small volume. The residue was partitioned between ethyl acetate and water. The organic layer was separated, and the aqueous layer was extracted with ethyl acetate twice. The organic extracts were combined, washed with water, brine, dried over MgSO₄, and concentrated to give rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (19 mg, 78%).

MS (ES⁺) m/z Calcd for C₃₀H₂₉BrClFN₄O₅+H [(M+H)⁺]: 659. found: 659.

### Example 79

### Preparation of rac-(2S,3S,4S,5R)-6′-bromo-4-(3-chloro-2-fluorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide

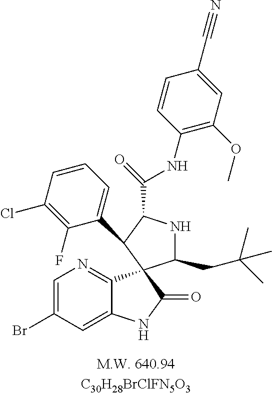

To a solution of E/Z-6-bromo-3-(3-chloro-2-fluoro-benzylidene)-1,3-dihydro-pyrrolo[3,2-b]pyridin-2-one (Example 76, 0.2 g, 0.57 mmol) in tetrahydrofuran (2 mL) was added anhydrous LiOH (27 mg, 1.1 mmol). The mixture was warmed to 40° C. and stirred for 10 min. N-(4-cyano-2-methoxy-phenyl)-2-[3,3-dimethyl-but-(E)-ylideneamino]-acetamide (Example 61, 0.24 g, 0.85 mmol) was added in one portion. The reaction mixture was stirred at 40° C. for 1 h. The mixture was cooled to room temperature and filtered through a short pad of silica gel. The silica gel was washed with ethyl acetate. The filtrate was concentrated. The resulting precipitate was filtered and collected to give rac-(2S,3S,4S,5R)-6′-bromo-4-(3-chloro-2-fluorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide as a yellow solid (80 mg, 22%). MS (ES⁺) m/z Calcd for C₃₀H₂₈BrClFN₅O₃+H [(M+H)⁺]: 640. found: 640.

### Example 80

### Preparation of rac-(2S,3S,4S,5R)-6′-bromo-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-b]pyridine]-5-carboxamide

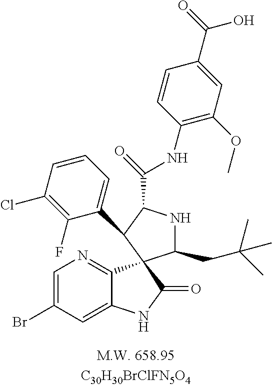

To a solution of rac-(2S,3S,4S,5R)-6′-bromo-4-(3-chloro-2-fluorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide (Example 79, 70 mg, 0.11 mmol) in DMSO (0.5 mL) at 0° C. was added an aqueous solution (30%) of H₂O₂ (Aldrich, 0.19 g, 1.6 mmol), followed by the addition of aqueous solution (1 N) of NaOH (0.54 mL, 0.54 mmol). The reaction mixture was stirred at 0° C. for 1 h. The mixture was partitioned between ethyl acetate and saturated aqueous Na₂SO₃ solution. The organic layer was separated, washed with water, brine, dried over MgSO₄, and concentrated to give rac-(2S,3S,4S,5R)-6′-bromo-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-b]pyridine]-5-carboxamide as a off white solid (56 mg, 78%). MS (ES⁺) m/z Calcd for C₃₀H₃₀BrClFN₅O₄+H [(M+H)⁺]: 658. found: 658.

### Example 81

### Preparation of rac-(2S,3S,4S,5R)-6′-bromo-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide

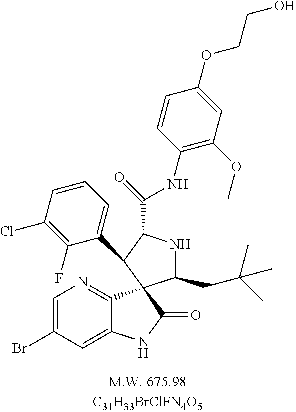

To a solution of E/Z-6-bromo-3-(3-chloro-2-fluoro-benzylidene)-1,3-dihydro-pyrrolo[3,2-b]pyridin-2-one (Example 76, 0.2 g, 0.57 mmol) in tetrahydrofuran (2 mL) was added anhydrous LiOH (27 mg, 1.1 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.31 g, 0.85 mmol) was added in one portion. The reaction mixture was stirred at 40° C. for 3 h. Then an aqueous solution (1 N) of NaOH (1 mL, 1 mmol) was added, and the reaction mixture was stirred at 40° C. for 1 h. The mixture was poured into water, and extracted with ethyl acetate three time. The combined extract was washed with water, brine, dried over MgSO₄, and concentrated. The residue was purified by chromatography (0-100% EtOAc in dichloromethane) to give rac-(2S,3S,4S,5R)-6′-bromo-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide as a light yellow solid (65 mg, 17%).

MS (ES⁺) m/z Calcd for C₃₁H₃₃BrClFN₄O₅+H [(M+H)⁺]: 675. found: 675.

### Example 82

### Preparation of intermediate E/Z-3-(3-chloro-2-fluoro-benzylidene)-6-fluoro-1,3-dihydro-pyrrolo[3,2-b]pyridin-2-one

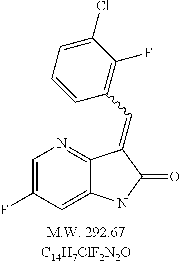

To the mixture of 6-fluoro-4-aza-2-oxindole (Sinova, 1 g, 4.6 mmol) and 3-chloro-2-fluorobenzaldehyde (Oakwood Products, 1.2 g, 7.6 mmol) in methanol (50 mL) was added piperidine (Aldrich, 2 g, 24 mmol) dropwise. The reaction mixture was stirred at room temperature for 10 h. Then the mixture was filtered. The resulting precipitate was collected to give the first batch of desired product. The filtrate was concentrated. The residue was purified by chromatography (20-40% EtOAc in hexanes) to give the second batch of desire product. The two batches were combined to give E/Z-3-(3-chloro-2-fluoro-benzylidene)-6-fluoro-1,3-dihydro-pyrrolo[3,2-b]pyridin-2-one as a yellow solid (Yield 1.1 g, 82%).

### Example 83

### Preparation of methyl rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoate

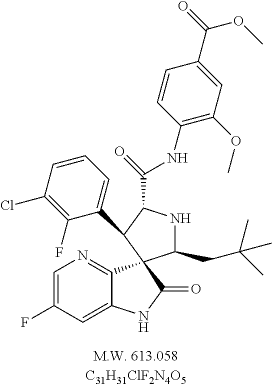

To a solution of E/Z-3-(3-chloro-2-fluoro-benzylidene)-6-fluoro-1,3-dihydro-pyrrolo[3,2-b]pyridin-2-one (Example 82, 0.3 g, 1 mmol) in tetrahydrofuran (10 mL) was added anhydrous LiOH (25 mg, 1 mmol). The mixture was warmed to 40° C. and stirred for 10 min. 4-{2-[3,3-dimethyl-but-(E)-ylideneamino]-acetylamino}-3-methoxy-benzoic acid methyl ester (Example 3, 0.33 g, 1 mmol) was added in one portion. The reaction mixture was stirred at 40° C. for 24 h. The mixture was cooled to room temperature and filtered. The resulting precipitate was collected, washed with ethyl acetate, and dried to give the first batch of desired product. The filtrate was concentrated, and the residue was purified by chromatography (5-10% EtOAc in dichlormethane) to give the second batch of desired product. The two batches were combined to give methyl rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (0.21 g, 33%). MS (ES⁺) m/z Calcd for C₃₁H₃₁ClF₂N₄O₅+H [(M+H)⁺]: 613. found: 613.

### Example 84

### Preparation of rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoic acid

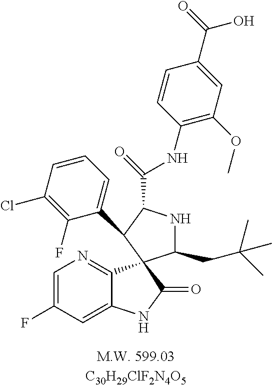

To a solution of methyl rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoate (Example 83, 0.21 g, 0.34 mmol) in tetrahydrofuran (8 mL) was added an aqueous solution (1 N) of NaOH (2 mL, 2 mmol). The reaction mixture was stirred at 60° C. for 24 h. The “pH” of the mixture was adjusted to 3 by aqueous HCl solution, then concentrated to a small volume. The residue was partitioned between ethyl acetate and water. The organic layer was separated, and the aqueous layer was extracted with ethyl acetate twice. The organic extracts were combined, washed with water, brine, dried over MgSO₄, and concentrated to give rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (0.19 g, 93%). MS (ES⁺) m/z Calcd for C₃₀H₂₉ClF₂N₄O₅+H [(M+H)⁺]: 599. found: 599.

### Example 85

### Preparation of rac-(2S,3S,4S,5R)-4-(3-chloro-2-fluorophenyl)-N-(4-cyano-2-methoxyphenyl)-6′-fluoro-2-neopentyl-2′-oxo-1′,2′-dihydrospiro[pyrrolidine-3,3′-pyrrolo[3,2-b]pyridine]-5-carboxamide

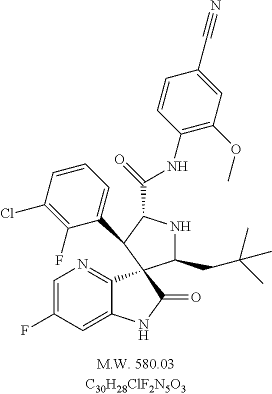

To a solution of E/Z-3-(3-chloro-2-fluoro-benzylidene)-6-fluoro-1,3-dihydro-pyrrolo[3,2-b]pyridin-2-one (Example 82, 0.18 g, 0.62 mmol) in tetrahydrofuran (2 mL) was added anhydrous LiOH (30 mg, 1.2 mmol). The mixture was warmed to 40° C. and stirred for 10 min. N-(4-cyano-2-methoxy-phenyl)-2-[3,3-dimethyl-but-(E)-ylideneamino]-acetamide (Example 61, 0.27 g, 0.92 mmol) was added in one portion. The reaction mixture was stirred at 40° C. for 1 h. The mixture was cooled to room temperature and filtered through a short pad of silica gel. The silica gel was washed with ethyl acetate. The filtrate was concentrated. The resulting precipitate was filtered and collected to give rac-(2S,3S,4S,5R)-4-(3-chloro-2-fluorophenyl)-N-(4-cyano-2-methoxyphenyl)-6′-fluoro-2-neopentyl-2′-oxo-1′,2′-dihydrospiro[pyrrolidine-3,3′-pyrrolo[3,2-b]pyridine]-5-carboxamide as a white solid (40 mg, 11%). MS (ES⁺) m/z Calcd for C₃₀H₂₈ClF₂N₅O₃+H [(M+H)⁺]: 580. found: 580.

### Example 86

### Preparation of rac-(2S,3S,4S,5R)-N-(4-carbamoyl-2-methoxyphenyl)-4-(3-chloro-2-fluorophenyl)-6′-fluoro-2-neopentyl-2′-oxo-1′,2′-dihydrospiro[pyrrolidine-3,3′-pyrrolo[3,2b]pyridine]-5-carboxamide

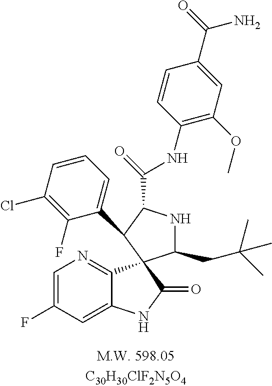

To a solution of rac-(2S,3S,4S,5R)-4-(3-chloro-2-fluorophenyl)-N-(4-cyano-2-methoxyphenyl)-6′-fluoro-2-neopentyl-2′-oxo-1′,2′-dihydrospiro[pyrrolidine-3,3′-pyrrolo[3,2-b]pyridine]-5-carboxamide (Example 85, 30 mg, 0.52 mmol) in DMSO (0.3 mL) at 0° C. was added an aqueous solution (30%) of H₂O₂ (Aldrich, 0.088 g, 0.78 mmol), followed by the addition of aqueous solution (1 N) of NaOH (0.26 mL, 0.26 mmol). The reaction mixture was stirred at 0° C. for 1 h. The mixture was partitioned between ethyl acetate and saturated aqueous Na₂SO₃ solution. The organic layer was separated, washed with water, brine, dried over MgSO₄, and concentrated to give rac-(2S,3S,4S,5R)-N-(4-carbamoyl-2-methoxyphenyl)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-carboxamide as an off white solid (24 mg, 78%). MS (ES⁺) m/z Calcd for C₃₀H₃₀ClF₂N₅O₄+H [(M+H)⁺]: 598. found: 598.

### Example 87

### Preparation of rac-(2S,3S,4S,5R)-4-(3-chloro-2-fluorophenyl)-N-[4-(2-hydroxyethoxy)-2-methoxyphenyl]-6′-fluoro-2-neopentyl-2′-oxo-1′,2′-dihydrospiro[pyrrolidine-3,3′-pyrrolo[3,2b]pyridine]-5-carboxamide

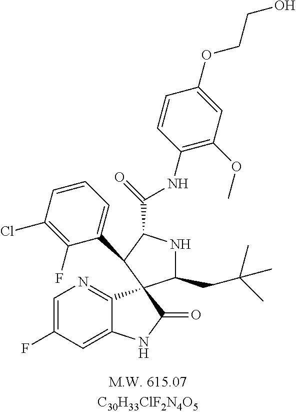

To a solution of E/Z-3-(3-chloro-2-fluoro-benzylidene)-6-fluoro-1,3-dihydro-pyrrolo[3,2-b]pyridin-2-one (Example 82, 0.15 g, 0.51 mmol) in tetrahydrofuran (2 mL) was added anhydrous LiOH (25 mg, 1 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.28 g, 0.77 mmol) prepared in was added in one portion. The reaction mixture was stirred at 40° C. for 3 h. Then an aqueous solution (1 N) of NaOH (1 mL, 1 mmol) was added, and the reaction mixture was stirred at 40° C. for 1 h. The mixture was poured into water, and extracted with ethyl acetate three time. The combined extract was washed with water, brine, dried over MgSO₄, and concentrated. The residue was purified by chromatography (0-100% EtOAc in dichloromethane) to give rac-(2S,3S,4S,5R)-4-(3-chloro-2-fluorophenyl)-N-[4-(2-hydroxyethoxy)-2-methoxyphenyl]-6′-fluoro-2-neopentyl-2′-oxo-1′,2′-dihydrospiro[pyrrolidine-3,3′-pyrrolo[3,2-b]pyridine]-5-carboxamide as a light yellow solid (55 mg, 17%). MS (ES⁺) m/z Calcd for C₃₁H₃₃ClF₂N₄O₅+H [(M+H)⁺]: 615. found: 615.

### Example 88

### Preparation of intermediate E/Z-2-chloro-5-(3-chloro-2-fluoro-benzylidene)-5,7-dihydro-pyrrolo[2,3-d]pyrimidin-6-one

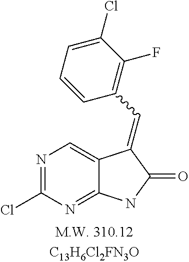

To a mixture of 2-chloro-5H-pyrrolo[2,3-d]pyrimidin-6(7H)-one (Molbridge, 0.5 g, 3.0 mmol) in acetic acid (8 mL) and aqueous concentrated HCl solution (37%, 2 mL) was added 3-chloro-2-fluorobenzaldehyde (Oakwood, 0.9 g, 5.7 mmol). The reaction mixture was stirred at room temperature for 24 h. The mixture was neutralized to “pH” 7-8 by aqueous saturated NaHCO₃ solution, then extracted with ethyl acetate several times. The combined organic extract was washed with water, brine, dried over MgSO₄, and concentrated to a small volume. The resulting precipitate was collected and dried to give the first batch of desired product. The filtrate was concentrated, and residue was purified by chromatography (20-40% EtOAc in hexanes) to give the second batch of desired product. The two batches were combined to give E/Z-2-chloro-5-(3-chloro-2-fluoro-benzylidene)-5,7-dihydro-pyrrolo[2,3-d]pyrimidin-6-one as a yellow solid (0.51 g, 56%).

### Example 89

### Preparation of rac-(2S,3R,4S,5R)-2′-chloro-4-(3-chloro-2-fluorophenyl)-N-[4-(2-hydroxyethoxy)-2-methoxyphenyl]-2-neopentyl-6′-oxo-6′,7′-dihydrospiro[pyrrolidine-3,5′-pyrrolo[2,3-d]pyrimidine]-5-carboxamide

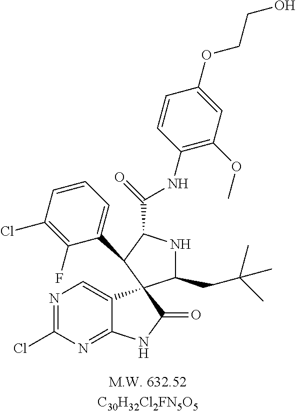

To a solution of E/Z-2-chloro-5-(3-chloro-2-fluoro-benzylidene)-5,7-dihydro-pyrrolo[2,3-d]pyrimidin-6-one (Example 88, 0.3 g, 0.97 mmol) in tetrahydrofuran (3 mL) was added anhydrous LiOH (23 mg, 0.97 mmol). The mixture was warmed to 40° C. and stirred for 10 min. 2-(4-{2-[3,3-dimethyl-but-(E)-ylideneamino]-acetylamino}-3-methoxy-phenoxy)-ethyl ester (Example 70, 0.53 g, 1.5 mmol) was added in one portion. The reaction mixture was stirred at 40° C. for 2 h. Then an aqueous solution (1 N) of NaOH (1.5 mL, 1.5 mmol) was added, and the reaction mixture was stirred at 40° C. for 1 h. The mixture was poured into water, and extracted with ethyl acetate three time. The combined extract was washed with water, brine, dried over MgSO₄, and concentrated. The residue was purified by chromatography (0-80% EtOAc in dichloromethane) to give rac-(2S,3R,4S,5R)-2′-chloro-4-(3-chloro-2-fluorophenyl)-N-[4-(2-hydroxyethoxy)-2-methoxyphenyl]-2-neopentyl-6′-oxo-6′,7′-dihydrospiro[pyrrolidine-3,5′-pyrrolo[2,3-d]pyrimidine]-5-carboxamide as a yellow solid (25 mg, 4%). MS (ES⁺) m/z Calcd for C₃₀H₃₂Cl₂FN₅O₅+H [(M+H)⁺]: 632. found: 632.

### Example 90

### Preparation of methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate

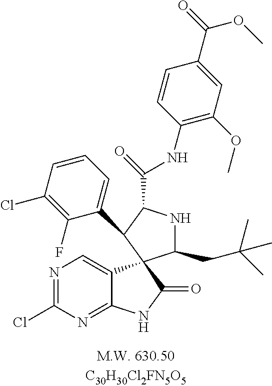

To a solution of E/Z-2-chloro-5-(3-chloro-2-fluoro-benzylidene)-5,7-dihydro-pyrrolo[2,3-d]pyrimidin-6-one (Example 88, 0.2 g, 0.65 mmol) in tetrahydrofuran (15 mL) was added anhydrous LiOH (15 mg, 0.65 mmol). The mixture was warmed to 40° C. and stirred for 10 min. 4-{2-[3,3-dimethyl-but-(E)-ylideneamino]-acetylamino}-3-methoxy-benzoic acid methyl ester (Example 3, 0.22 g, 0.68 mmol) was added in one portion. The reaction mixture was stirred at 40° C. for 5 h. The mixture was cooled to room temperature and concentrated. The residue was purified by chromatography (5-10% EtOAc in dichlormethane) to give methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate as a white solid (0.12 g, 28%). MS (ES⁺) m/z Calcd for C₃₀H₃₀Cl₂FN₅O₅+H [(M+H)⁺]: 630. found: 630.

### Example 91

### Preparation of methyl rac-4-{(2S,3S,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate

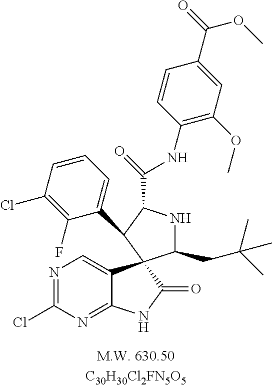

In the preparation of methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate as described in Example 90, methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate was obtained as another product by chromatography (5-10% EtOAc in dichlormethane): Yield 45 mg, 11%, a white solid. MS (ES⁺) m/z Calcd for C₃₀H₃₀Cl₂FN₅O₅+H [(M+H)⁺]: 630. found: 630.

### Example 92

### Preparation of rac-4-{(2S,3R,4S,5R)-2′-chloro-4-(3-chloro-2-fluorophenyl)-2-neopentyl-6′-oxo-6′,7-dihydrospiro[pyrrolidine-3,5′-pyrrolo[2,3-d]pyrimidine]-5-ylcarboxamido}-3-methoxybenzoic acid

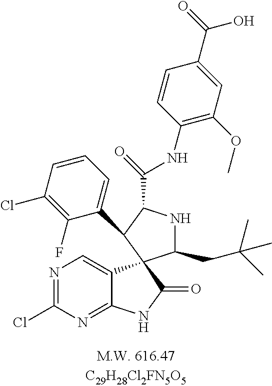

To a solution of methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate (Example 90, 98 mg, 0.16 mmol) in tetrahydrofuran (3 mL) was added an aqueous solution (1 N) of NaOH (1 mL, 1 mmol). The reaction mixture was stirred at 60° C. for 24 h. The “pH” of the mixture was adjusted to 3 by aqueous HCl solution, then concentrated to a small volume. The residue was partitioned between ethyl acetate and water. The organic layer was separated, and the aqueous layer was extracted with ethyl acetate twice. The organic extracts were combined, washed with water, brine, dried over MgSO₄, and concentrated to give rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoic acid as a white solid (90 mg, 94%). MS (ES⁺) m/z Calcd for C₂₉H₂₈Cl₂FN₅O₅+H [(M+H)⁺]: 616. found: 616.

### Example 93

### Preparation of rac-(2S,3R,4S,5R)-N-(4-carbamoyl-2-methoxyphenyl)-2′-chloro-4-(3-chloro-2-fluorophenyl)-2-neopentyl-6′-oxo-6′,7-dihydrospiro[pyrrolidine-3,5′-pyrrolo[2,3-d]pyrimidine]-5-carboxamide

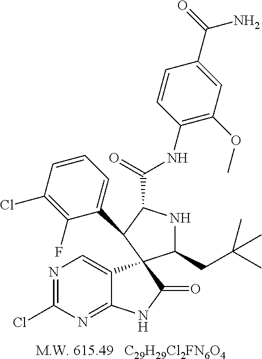

To a solution of rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoic acid (Example 92, 90 mg, 0.15 mmol) in anhydrous DMF (2 mL) were added EDCI (56 mg, 0.29 mmol), HOBt (39 mg, 0.29 mmol), NH₄Cl (77 mg, 1.5 mmol), and triethylamine (30 mg, 0.29 mmol) sequentially. The reaction mixture was heated at 68° C. for 1 h. The mixture was partitioned between ethyl acetate and water. The organic layer was separated, and aqueous layer was extracted with ethyl acetate twice. The combined organic extract was washed with water, brine, dried over MgSO₄, and concentrated. The residue was purified by chromatography (25-100% EtOAc in dichloromethane) to give rac-(2S,3R,4S,5R)-N-(4-carbamoyl-2-methoxyphenyl)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-carboxamide as a light yellow solid (67 mg, 75%). MS (ES⁺) m/z Calcd for C₂₉H₂₉Cl₂FN₆O₄+H [(M+H)⁺]: 615. found: 615.

### Example 94

### Preparation of intermediate 2-chloro-4,6-dihydro-thieno[3,2-b]pyrrol-5-one

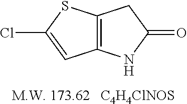

Step a: To a solution of 2,5-dichlorothiophene (Aldrich, 21 g, 137 mmol) in concentrated H₂SO₄ (59 mL) at 0° C. was added a fine powder form of NaNO₃ (28 g, 412 mmol) in one portion. The reaction mixture was stirred at 0° C. for 2 min when a brown fume began to appear. The reaction mixture was poured into the mixture of ice-water and ethyl acetate. The organic layer was separated, and aqueous layer was extracted with ethyl acetate. The combined organic extract was washed with water, brine, dried over MgSO₄, and concentrated. The residue was purified by chromatography (1% EtOAc in hexanes) to give 2,5-dichloro-3-nitrothiophene as a yellow oil (17 g, 63%).

Step b: To a solution of tert-butyl ethyl malonate (Alfa, 16.2 g, 86 mmol) in anhydrous DMSO (50 mL) were added NaH (Aldrich, 60%, 5.15 g, 129 mmol). The mixture was heated at 100° C. for 1 h, the cooled to room temperature. 2,5-Dichloro-3-nitrothiophene (17 g, 86 mmol) was added in one portion. The reaction mixture was heated at 60° C. for 2 h. The mixture was cooled to room temperature, and water and dilute aqueous HCl solution were slowly added. The mixture was extracted with ethyl acetate twice times. The combined organic extract was washed with water, brine, dried over MgSO₄, and concentrated. Trifluoroacetic acid (50 mL) was added. The reaction mixture was stirred at room temperature for 20 h. The mixture was concentrated. The residue was partitioned between ethyl acetate and saturated aqueous NaHCO₃ solution. The organic layer was separated, washed with water, brine, dried over MgSO₄, and concentrated. The residue was purified by chromatography (0-20% EtOAc in hexanes) to give ethyl 2-(5-chloro-3-nitrothiophen-2-yl)acetate as a brown oil (10 g, 61%).

Step c: To a solution of ethyl 2-(5-chloro-3-nitrothiophen-2-yl)acetate (10 g, 40 mmol) in methanol (200 mL) was added an aqueous solution (40 mL) of NH₄Cl (17 g, 320 mmol), followed by activated Zinc (Aldrich, 15.7 g, 240 mmol). The reaction mixture was stirred at room temperature for 2 h. The mixture was filtered through a short pad of celite. The mixture was concentrated. The residue was partitioned between ethyl acetate and water. The organic layer was separated, and aqueous layer was extracted with ethyl acetate. The combined organic extract was washed with water, brine, dried over MgSO₄, and concentrated. The residue was purified by chromatography (20-50% EtOAc in hexanes) to give ethyl 2-(3-amino-5-chlorothiophen-2-yl)acetate as a yellow oil (7 g, 80%).

Step d: To a flask charged with ethyl 2-(3-amino-5-chlorothiophen-2-yl)acetate (6.7 g, 31 mmol) was added anhydrous toluene (30 mL). The mixture was evaporated to dryness. The process was repeated three times. To the residue was added toluene (300 mL), and the temperature of the solution was lowered to 0° C. A toluene solution (2 N) of trimethylaluminum (38 mL, 76 mmol) was added. The reaction mixture was stirred at 10° C. for 0.5 h, then quenched by methanol (10 mL) slowly. The mixture was poured into saturated aqueous NH₄Cl solution, and extracted with ethyl acetate twice. The combined organic extract was dried over MgSO₄ and concentrated to give warmed to room temperature and stirred for 1 h. The mixture was concentrated. The residue was partitioned between ethyl acetate and water. The organic layer was separated, and aqueous layer was extracted with ethyl acetate. The combined organic extract was washed with water, saturated aqueous CuSO₄ solution, brine, dried over MgSO₄, and concentrated to give crude 2-chloro-4,6-dihydro-thieno[3,2-b]pyrrol-5-one as a black solid (5.4 g, 75%).

### Example 95

### Preparation of intermediate E/Z-2-chloro-6-(3-chloro-2-fluorobenzylidene)-4H-thieno[3,2-b]pyrrol-5(6H)-one

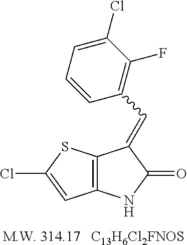

To a mixture of 2-chloro-4,6-dihydro-thieno[3,2-b]pyrrol-5-one (Example 94, 4.2 g, 24 mmol) in acetic acid (60 mL) and aqueous concentrated HCl solution (37%, 15 mL) was added 3-chloro-2-fluorobenzaldehyde (Oakwood, 7.5 g, 47 mmol). The reaction mixture was stirred at room temperature for 24 h. The mixture was filtered, and the resulting precipitate was collected, washed with ethyl acetate, and dried to give the first batch of desire product. The filtrate was concentrated, and the residue was neutralized to “pH” 7-8 by aqueous saturated NaHCO₃ solution, then extracted with ethyl acetate several times. The combined organic extract was washed with water, brine, dried over MgSO₄, and concentrated and residue was purified by chromatography (20-40% EtOAc in hexanes) to give the second batch of desired product. The two batches were combined to give E/Z-2-chloro-6-(3-chloro-2-fluorobenzylidene)-4H-thieno[3,2-b]pyrrol-5(6H)-one as a brown solid (5.1 g, 69%).

### Example 96

### Preparation of methyl rac-4-((2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate

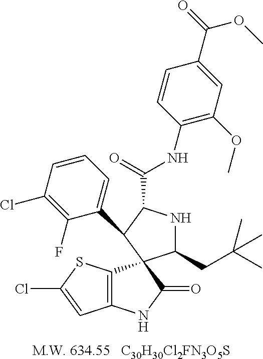

To a solution of E/Z-2-chloro-6-(3-chloro-2-fluorobenzylidene)-4H-thieno[3,2-b]pyrrol-5(6H)-one (Example 95, 0.37 g, 1.2 mmol) in tetrahydrofuran (10 mL) was added anhydrous LiOH (28 mg, 1.2 mmol). The mixture was warmed to 40° C. and stirred for 10 min. 4-{2-[3,3-dimethyl-but-(E)-ylideneamino]-acetylamino}-3-methoxy-benzoic acid methyl ester (Example 3, 0.4 g, 1.3 mmol) was added in one portion. The reaction mixture was stirred at 40° C. for 60 h. The mixture was cooled to room temperature and filtered through a short pad of silica gel. The silica gel was washed with ethyl acetate. The filtrate was concentrated. The residue was purified by chromatography (5-10% EtOAc in dichlormethane) to give methyl rac-4-((2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate as a brown solid (90 mg, 12%). MS (ES⁺) m/z Calcd for C₃₀H₃₀Cl₂FN₃O₅S+H [(M+H)⁺]: 634. found: 634.

### Example 97

### Preparation of methyl rac-4-((2S,3S,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate

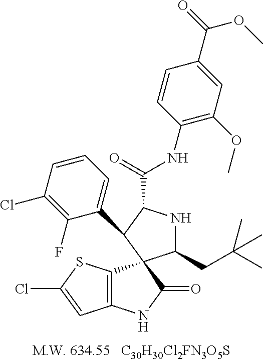

In the preparation of methyl rac-4-((2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate as described in Example 96, methyl rac-4-((2S,3S,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate was obtained as another product by chromatography (5-10% EtOAc in dichlormethane): Yield 30 mg, 4%, a brown solid. MS (ES⁺) m/z Calcd for C₃₀H₃₀Cl₂FN₃O₅S+H [(M+H)⁺]: 634. found: 634.

### Example 98

### Preparation of rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid

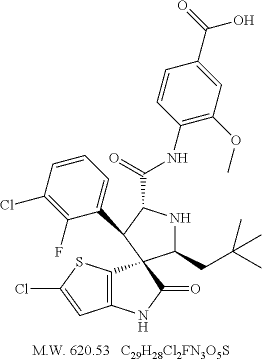

To a solution of methyl rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoate (Example 96, 90 mg, 0.14 mmol) in tetrahydrofuran (3 mL) was added an aqueous solution (1 N) of NaOH (1 mL, 1 mmol) and methanol (1 mL). The reaction mixture was stirred at room temperature for 18 h. The “pH” of the mixture was adjusted to 3-6 by aqueous HCl solution. The mixture was concentrated to a small volume, then partitioned between ethyl acetate and water. The organic layer was separated, and the aqueous layer was extracted with ethyl acetate twice. The organic extracts were combined, washed with water, brine, dried over MgSO₄, and concentrated to give rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid as a off white solid (75 mg, 85%). MS (ES⁺) m/z Calcd for C₂₉H₂₈Cl₂FN₃O₅S+H [(M+H)⁺]: 620. found: 620.

### Example 99

### Preparation of chiral 4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid

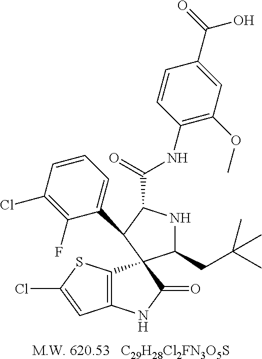

Rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid (Example 98, 0.15 g) was separated by chiral SFC chromatography to provide chiral 4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid as a yellow solid (39 mg, 24%) and chiral 4-{(2R,3S,4S,5S)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid as an off white solid (41 mg, 25%). MS (ES⁺) m/z Calcd for C₂₉H₂₈Cl₂FN₃O₅S+H [(M+H)⁺]: 620. found: 620.

### Example 100

### Preparation of rac-(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide

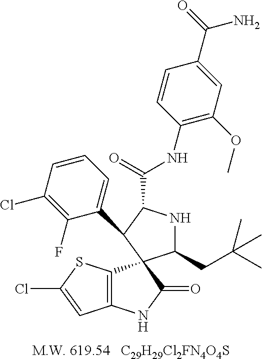

To a solution of rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid (220 mg, 0.36 mmol) in anhydrous DMF (2 mL) were added EDCI (Example 98, 136 mg, 0.71 mmol), HOBt (96 mg, 0.71 mmol), NH₄Cl (188 mg, 3.55 mmol), and triethylamine (72 mg, 0.71 mmol) sequentially. The reaction mixture was heated at 68° C. for 1 h. The mixture was cooled to room temperature, then partitioned between ethyl acetate and water. The organic layer was separated, and aqueous layer was extracted with ethyl acetate twice. The combined organic extract was washed with water, brine, dried over MgSO₄, and concentrated. The residue was purified by chromatography (25-100% EtOAc in dichloromethane) to give rac-(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide as a light brown solid (170 mg, 77%). MS (ES⁺) m/z Calcd for C₂₉H₂₉Cl₂FN₄O₄S+H [(M+H)⁺]: 619. found: 619.

### Example 101

### Preparation of chiral(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide

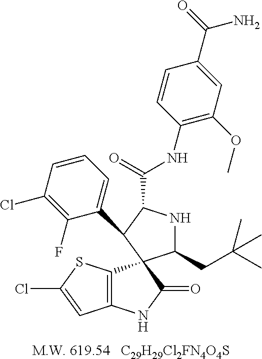

Rac-(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide (Example 100, 0.17 g) was separated by chiral SFC chromatography to provide chiral(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide as a light yellow solid (70 mg, 41%) and chiral(2R,3S,4S,5S)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide as a light yellow solid (68 mg, 40%). MS (ES⁺) m/z Calcd for C₂₉H₂₉Cl₂FN₄O₄S+H [(M+H)⁺]: 619. found: 619.

### Example 102

### Preparation of rac-(2S,3S,4R,5R)-2′-chloro-4-(3-chloro-2-fluorophenyl)-N-(4-cyano-2-methoxyphenyl)-2-neopentyl-5′-oxo-4′,5′-dihydrospiro[pyrrolidine-3,6′-thieno[3,2-b]pyrrole]-5-carboxamide

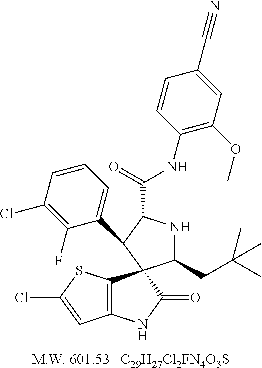

To a solution of E/Z-2-chloro-6-(3-chloro-2-fluorobenzylidene)-4H-thieno[3,2-b]pyrrol-5(6H)-one (Example 95, 0.15 g, 0.48 mmol) in tetrahydrofuran (1 mL) was added anhydrous LiOH (23 mg, 0.96 mmol). The mixture was warmed to 40° C. and stirred for 10 min. N-(4-cyano-2-methoxy-phenyl)-2-[3,3-dimethyl-but-(E)-ylideneamino]-acetamide (Example 61, 0.23 g, 0.79 mmol) was added in one portion. The reaction mixture was stirred at 40° C. for 1 h. The mixture was cooled to room temperature and filtered through a short pad of silica gel. The silica gel was washed with ethyl acetate. The filtrate was concentrated. The residue was purified by chromatography (0-20% EtOAc in dichlormethane) to give rac-(2S,3S,4R,5R)-2′-chloro-4-(3-chloro-2-fluorophenyl)-N-(4-cyano-2-methoxyphenyl)-2-neopentyl-5′-oxo-4′,5′-dihydrospiro[pyrrolidine-3,6′-thieno[3,2-b]pyrrole]-5-carboxamide as a purple solid (40 mg, 14%). MS (ES) m/z Calcd for C₂₉H₂₇Cl₂FN₄O₃S+H [(M+H)⁺]: 601. found: 601.

### Example 103

### Preparation of rac-(2S,3S,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide

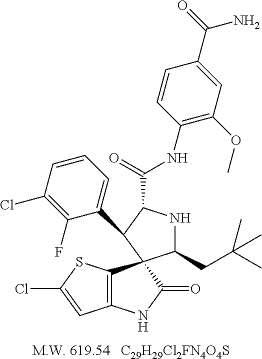

To a solution of rac-(2S,3S,4R,5R)-2′-chloro-4-(3-chloro-2-fluorophenyl)-N-(4-cyano-2-methoxyphenyl)-2-neopentyl-5′-oxo-4′,5′-dihydrospiro[pyrrolidine-3,6′-thieno[3,2-b]pyrrole]-5-carboxamide (Example 102, 28 mg, 0.47 mmol) in DMSO (0.2 mL) at 0° C. was added an aqueous solution (30% Aldrich) of H₂O₂ (0.079 g, 0.7 mmol), followed by the addition of aqueous solution (1 N) of NaOH (0.23 mL, 0.23 mmol). The reaction mixture was stirred at 0° C. for 1 h. The mixture was partitioned between ethyl acetate and saturated aqueous Na₂SO₃ solution. The organic layer was separated, washed with water, brine, dried over MgSO₄, and concentrated to give rac-(2S,3S,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide as a purple solid (Yield 25 mg, 87%). MS (ES⁺) m/z Calcd for C₂₉H₂₉Cl₂FN₄O₄S+H [(M+H)⁺]: 619. found: 619.

### Example 104

### Preparation of intermediate rac-(2S,3R,4R,5R)-tert-butyl 2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylate

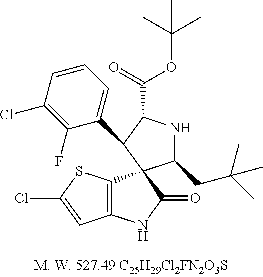

To a solution of E/Z-2-chloro-6-(3-chloro-2-fluorobenzylidene)-4H-thieno[3,2-b]pyrrol-5(6H)-one (Example 95, 0.9 g, 2.9 mmol) in tetrahydrofuran (20 mL) was added anhydrous LiOH (97 mg, 4.1 mmol). The mixture was warmed to 40° C. and stirred for 10 min. [3,3-dimethyl-but-(E)-ylideneamino]-acetic acid tert-butyl ester (Example 5, Step a, 1.8 g, 8.4 mmol) was added in one portion. The reaction mixture was stirred at 40° C. for 66 h. The mixture was cooled to room temperature and filtered through a short pad of silica gel. The silica gel was washed with ethyl acetate. The filtrate was concentrated. The residue was purified by chromatography (10-33% EtOAc in dichlormethane) to give rac-(2S,3R,4R,5R)-tert-butyl 2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylate as a white solid (0.18 g, 12%).

### Example 105

### Preparation of intermediate rac-(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylic acid trifluoroacetic acid

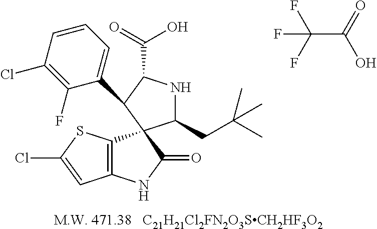

A solution of rac-(2S,3R,4R,5R)-tert-butyl 2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylate (Example 104, 64 mg, 0.12 mmol) in dichloromethane (2 mL) was added trifluoroacetic acid (3 mL). The reaction mixture was stirred at room temperature for 24 h, then concentrated. The residue was then triturated with ethyl ether and hexanes, concentrated, dried in vacuo to give rac-(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylic acid trifluoroacetic acid as an off white solid (71 mg, 100%).

### Example 106

### Preparation of intermediate 4-[2-(tert-butyl-dimethyl-silanyloxy)-ethoxy]-2-methoxy-phenylamine

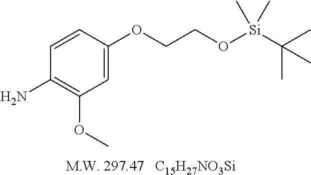

A suspension of tert-butyl-[2-(3-methoxy-4-nitro-phenoxy)-ethoxy]-dimethyl-silane (Example 70, Step b, 1 g, 3.05 mmol) and Pd/C (Aldrich, 10%, 0.1 g) in ethyl acetate (25 mL) was vigorously shaken in a Parr under atmosphere of H₂ (50 psi) for 0.5 h. The mixture was filtered through a short pad of celite. The filtrate was concentrated to give 4-[2-(tert-butyl-dimethyl-silanyloxy)-ethoxy]-2-methoxy-phenylamine as a light yellow oil (0.9 g, 99%).

### Example 107

### Preparation of rac-(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide

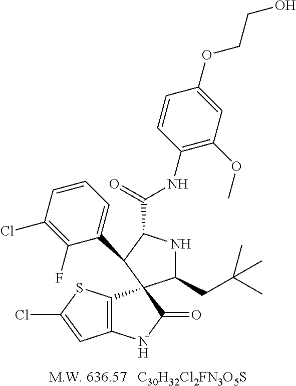

To a solution of rac-(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxylic acid trifluoroacetic acid (Example 105, 0.18 g, 0.31 mmol) in dichloromethane (5 mL) was added diisopropylethylamine (0.2 g, 1.5 mmol), diphenylphosphinic chloride (Aldrich, 0.15 g, 0.61 mmol) respectively. The mixture was stirred at room temperature for 8 min, then 4-(2-(tert-butyldimethylsilyloxy)ethoxy)-2-methoxyaniline (0.14 g, 0.46 mmol) was added. The reaction mixture was stirred at room temperature for 3 h. The mixture was concentrated. The residue was dissolved into tetrahydrofuran (5 mL), and an aqueous solution (1 N) of HCl (1 mL, 1 mmol) was added. The reaction mixture was stirred at room temperature for 1 h, then concentrated. The residue was partitioned between ethyl acetate and water. The organic layer was separated, and aqueous layer was extracted with ethyl acetate twice. The combined organic extract was washed with water, brine, dried over Na₂SO₄, then concentrated. The residue was purified by chromatography (10-100% of EtOAc in CH₂Cl₂) to give rac-(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide as a yellow solid (0.1 g, 51%).

HRMS (ES⁺) m/z Calcd for C₃₀H₃₂Cl₂FN₃O₅S+H [(M+H)⁺]: 636. found: 636.

### Example 108

### Preparation of chiral(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide

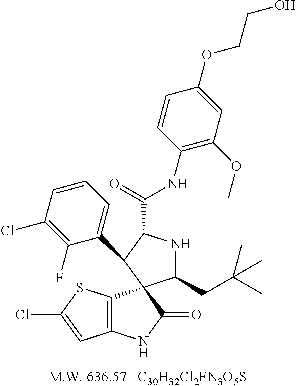

Rac-(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide (Example 107, 93 mg) was separated by chiral SFC chromatography to provide chiral(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide as a off white solid (25 mg, 27%) and chiral(2R,3S,4S,5S)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide as an off white solid (33 mg, 36%). MS (ES⁺) m/z Calcd for C₃₀H₃₂Cl₂FN₃O₅S+H [(M+H)⁺]: 637. found: 637.

### Example 109

### Preparation of intermediate E/Z-3-(3-Chloro-2-fluoro-benzylidene)-6-methoxy-1,3-dihydro-pyrrolo[3,2-c]pyridin-2-one

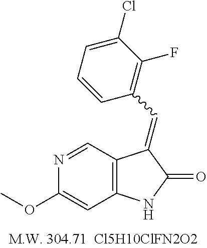

In a manner similar to the method described in Example 12, 6-methoxy-1H-pyrrolo[3,2-c]pyridin-2(3H)-one (Sinova, 489 mg, 2.98 mmol) was reacted with 3-chloro-2-fluorobenzaldehyde (Oakwood Products, 1.42 g, 8.94 mmol) and piperidine (Lancaster, 1.03 g, 1.2 mL, 12.1 mmol) in methanol (20 mL) to give E/Z-3-(3-Chloro-2-fluoro-benzylidene)-6-methoxy-1,3-dihydro-pyrrolo[3,2-c]pyridin-2-one as a light yellow solid (835 mg, 92%). MS (ES⁺) m/z [(M+H)⁺]: 305

### Example 110

### Preparation of methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate

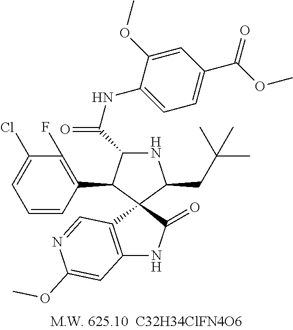

In a manner similar to the method described in Example 13, E/Z-3-(3-Chloro-2-fluoro-benzylidene)-6-methoxy-1,3-dihydro-pyrrolo[3,2-c]pyridin-2-one (300 mg, 0.985 mmol, Example X1) was reacted with anhydrous LiOH (14 mg, 0.585 mmol) and (E)-methyl 4-(2-(3,3-dimethylbutylideneamino)acetamido)-3-methoxybenzoate (329 mg, 1.03 mmol, Example 3) at 40° C. for 23 h to give methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate as a white solid (188 mg, 30%). MS (ES⁺) m/z [(M+H)⁺]: 625

### Example 111

### Preparation of rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoic acid

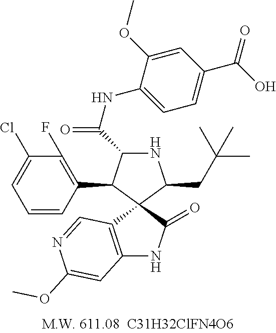

In a manner similar to the method described in Example 14, methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate (Example 110, 86 mg, 0.14 mmole) was reacted with LiOH hydrate (57 mg, 1.36 mmol) in water to give rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoic acid as a white solid (76 mg, 98%). MS (ES⁺) m/z [(M+H)⁺]: 611

### Example 112

### Preparation of intermediate E/Z-3-(3-Chloro-2-fluoro-benzylidene)-6-hydroxy-1,3-dihydro-pyrrolo[3,2-c]pyridin-2-one

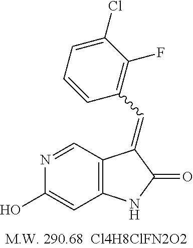

In a manner similar to the method described in Example 12, 6-hydroxy-1H-pyrrolo[3,2-c]pyridin-2(3H)-one (Sinova, 700 mg, 4.66 mmol) was reacted with 3-chloro-2-fluorobenzaldehyde (Oakwood Products, 2.20 g, 13.9 mmol) and piperidine (Lancaster, 1.59 g, 18.7 mmol) to give E/Z-3-(3-Chloro-2-fluoro-benzylidene)-6-hydroxy-1,3-dihydro-pyrrolo[3,2-c]pyridin-2-one as a light brown solid (683 mg, 50%). MS (ES⁺) m/z [(M+H)⁺]: 291

### Example 113

### Preparation of methyl rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoate

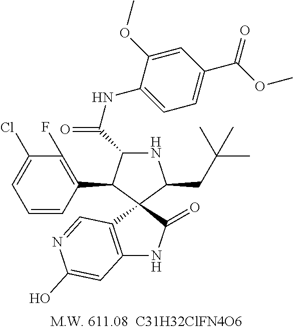

In a manner similar to the method described in Example 13, E/Z-3-(3-Chloro-2-fluoro-benzylidene)-6-hydroxy-1,3-dihydro-pyrrolo[3,2-c]pyridin-2-one (300 mg, 0.985 mmol, Example 112) was reacted with anhydrous LiOH (17.5 mg, 0.73 mmol) and (E)-methyl 4-(2-(3,3-dimethylbutylideneamino)acetamido)-3-methoxybenzoate (405 mg, 1.26 mmol, Example 3) at 40° C. for 23 h to give methyl rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoate as a white solid (253 mg, 32%). MS (ES⁺) m/z [(M+H)⁺]: 611

### Example 114

### Preparation of rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoic acid

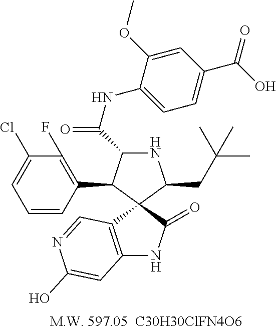

In a manner similar to the method described in Example 14, methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate (Example 113, 99 mg, 0.16 mmole) was reacted with LiOH hydrate (62 mg, 1.49 mmol) in water to give rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoic acid as a white solid (87 mg, 90%). MS (ES⁺) m/z [(M+H)⁺]: 597

### Example 115

### Preparation of rac-(2S,3R,4S,5R)-N-(4-carbamoyl-2-methoxyphenyl)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-carboxamide

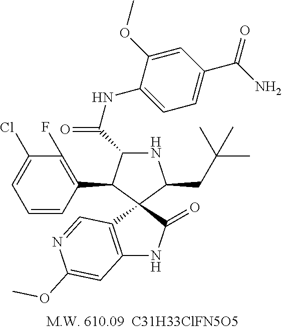

In a manner similar to the method described in Example 19, rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoic acid (40 mg, 0.065 mmol, Example 111) was reacted with 1,1′-carbonyldiimidazole (Aldrich, 52 mg, 0.32 mmol) in THF (3 mL) followed by work-up with ammonium hydroxide (310 mg, 8.84 mmol) to give rac-(2S,3R,4S,5R)-N-(4-carbamoyl-2-methoxyphenyl)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-carboxamide as a white solid (11 mg, 26%). MS (ES⁺) m/z [(M+H)⁺]: 610

### Example 116

### Preparation of rac-2-(4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxyphenoxy)ethyl acetate

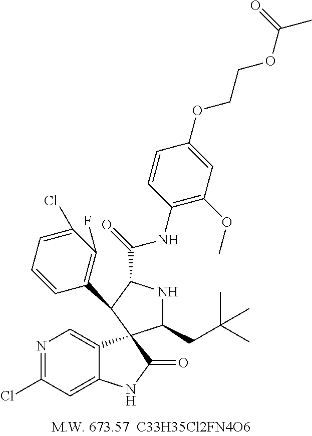

In a manner similar to the method described in Example 71, E/Z-6-Chloro-3-(3-chloro-2-fluoro-benzylidene)-1,3-dihydro-pyrrolo[3,2-c]pyridin-2-one (Example 23, 245 mg, 0.795 mmole) was reacted with 2-(4-{2-[3,3-dimethyl-but-(E)-ylideneamino]-acetylamino}-3-methoxy-phenoxy)-ethyl ester (Example 70, 316 mg, 0.867 mmol) and anhydrous LiOH (14 mg, 0.60 mmol) to give rac-2-(4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxyphenoxy)ethyl acetate as an off-white solid. MS (ES⁺) m/z [(M+H)⁺]: 655

### Example 117

### Preparation of rac-(2S,3R,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-c]pyridine]-5-carboxamide

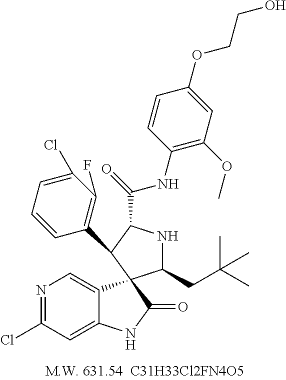

In a manner similar to the method described in Example 72, rac-2-(4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxyphenoxy)ethyl acetate was reacted with aqueous NaOH in THF to give rac-(2S,3R,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-c]pyridine]-5-carboxamide as a white solid. MS (ES⁺) m/z [(M+H)⁺]: 631.

### Example 118

### Preparation of intermediate E/Z-3-(3-Chloro-2-fluoro-benzylidene)-6-methyl-1,3-dihydro-pyrrolo[3,2-c]pyridin-2-one

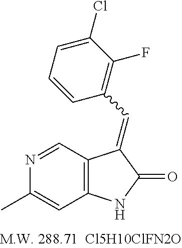

In a manner similar to the method described in Example 12, 6-methyl-1H-pyrrolo[3,2-c]pyridin-2(3H)-one (Sinova, 750 mg, 5.06 mmol) was reacted with 3-chloro-2-fluorobenzaldehyde (Oakwood Products, 2.40 g, 15.1 mmol) and piperidine (Lancaster, 172 g, 20.2 mmol) to give E/Z-3-(3-Chloro-2-fluoro-benzylidene)-6-methyl-1,3-dihydro-pyrrolo[3,2-c]pyridin-2-one as a light brown solid (623 mg, 41%). MS (ES⁺) m/z [(M+H)⁺]: 289

### Example 119

### Preparation of methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate

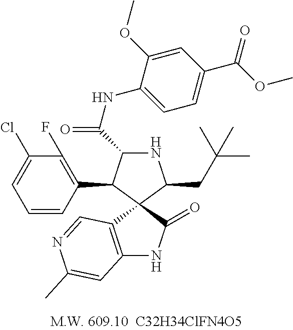

In a manner similar to the method described in Example 13, E/Z-3-(3-Chloro-2-fluoro-benzylidene)-6-methyl-1,3-dihydro-pyrrolo[3,2-c]pyridin-2-one (318 mg, 1.10 mmol, Example 118) was reacted with anhydrous LiOH (19 mg, 0.81 mmol) and (E)-methyl 4-(2-(3,3-dimethylbutylideneamino)acetamido)-3-methoxybenzoate (393 mg, 1.23 mmol, Example 3) at 40° C. for 23 h to give methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate as a white solid (146 mg, 21%). MS (ES⁺) m/z [(M+H)⁺]: 609

### Example 120

### Preparation of rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoic acid

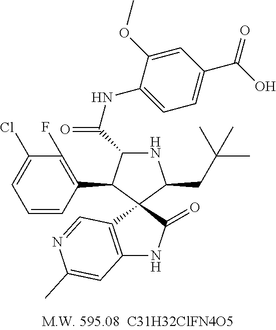

In a manner similar to the method described in Example 14, methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate (Example X11, 136 mg, 0.22 mmole) was reacted with LiOH hydrate (78 mg, 1.87 mmol) in water to give rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoic acid as a white solid (118 mg, 88%). MS (ES⁺) m/z [(M+H)⁺]: 595

### Example 121

### Preparation of rac-(2S,3R,4S,5R)-N-(4-carbamoyl-2-methoxyphenyl)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-carboxamide

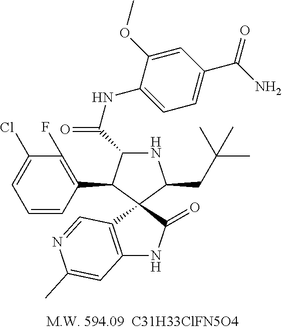

In a manner similar to the method described in Example 19, rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoic acid (21 mg, 0.034 mmol, Example 120) was reacted with 1,1′-carbonyldiimidazole (Aldrich, 27 mg, 0.165 mmol) in THF (3 mL) followed by work-up with ammonium hydroxide (0.18 mL mg, 4.62 mmol) to give rac-(2S,3R,4S,5R)-N-(4-carbamoyl-2-methoxyphenyl)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-carboxamide as a white solid (20 mg, 97%). MS (ES⁺) m/z [(M+H)⁺]: 594

### Example 122

### Preparation of intermediate E/Z-7-Chloro-3-(3-chloro-2-fluoro-benzylidene)-1,3-dihydro-pyrrolo[3,2-b]pyridin-2-one

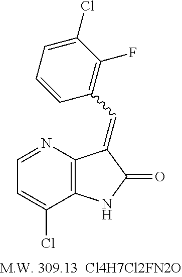

In a manner similar to the method described in Example 12, 7-chloro-1H-pyrrolo[3,2-b]pyridin-2(3H)-one (Sinova, 500 mg, 2.97 mmol) was reacted with 3-chloro-2-fluorobenzaldehyde (Oakwood Products, 1.010 g, 8.9 mmol) and piperidine (Lancaster, 1.01 g, 11.9 mmol) to give E/Z-7-Chloro-3-(3-chloro-2-fluoro-benzylidene)-1,3-dihydro-pyrrolo[3,2-b]pyridin-2-one as a light brown solid (615 mg, 67%). MS (ES⁺) m/z [(M+H)⁺]: 309

### Example 123

### Preparation of methyl rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate

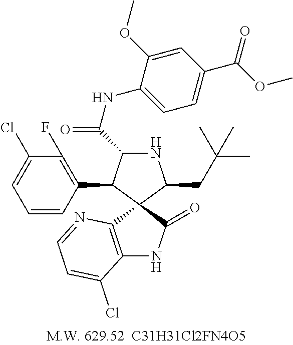

In a manner similar to the method described in Example 13, E/Z-7-Chloro-3-(3-chloro-2-fluoro-benzylidene)-1,3-dihydro-pyrrolo[3,2-b]pyridin-2-one (301 mg, 0.974 mmol, Example 122) was reacted with anhydrous LiOH (17 mg, 0.697 mmol) and (E)-methyl 4-(2-(3,3-dimethylbutylideneamino)acetamido)-3-methoxybenzoate (326 mg, 1.02 mmol, Example 3) at 40° C. for 23 h to give methyl rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate as a white solid (38 mg, 6%). MS (ES⁺) m/z [(M+H)⁺]: 629

### Example 124

### Preparation of rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid

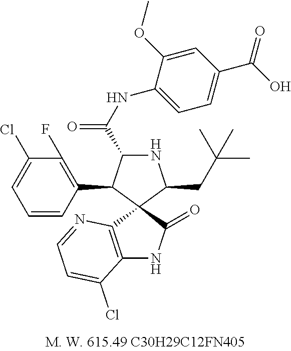

In a manner similar to the method described in Example 14, methyl rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate (Example 123, 32 mg, 0.052 mmole) was reacted with LiOH hydrate (17 mg, 0.42 mmol) in water to give rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid as a white solid (30 mg, 95%). MS (ES⁺) m/z [(M+H)⁺]: 615

### Example 125

### Preparation of rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-carboxamide

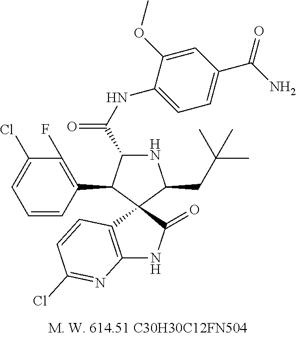

In a manner similar to the method described in Example 19, rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid (16 mg, 0.026 mmol, Example 124) was reacted with 1,1′-carbonyldiimidazole (Aldrich, 19 mg, 0.115 mmol) in THF (3 mL) followed by work-up with ammonium hydroxide (126 mg, 3.6 mmol) to give rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-carboxamide as a white solid (11 mg, 65%). MS (ES⁺) m/z [(M+H)⁺]: 614

### Example 126

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53. Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing: 90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at

665 and 615 nm (Victor 5, Perkin Elmer Wallace). If not specified, the reagents were purchased from Sigma Chemical Co.

Activity data for some of the Example compounds expressed as IC₅₀: bsa: 0.02% are as follows:

| Example | |
|---|---|
| Number | IC₅₀: bsa: 0.02% |
| 6 | 0.0324 |
| 13 | 0.0303 |
| 28 | 0.0105 |
| 35 | 0.0145 |
| 39 | 0.0084 |
| 47 | 0.0345 |
| 52 | 0.0050 |
| 56A | 0.0050 |
| 63 | 0.0146 |
| 66 | 1.8332 |
| 69 | 0.0129 |
| 75 | 0.0064 |
| 83 | 0.0195 |
| 86 | 0.0187 |
| 101 | 0.0046 |
| 89 | 0.0117 |
| 91 | 0.0107 |
| 96 | 0.0227 |
| 99 | 0.0054 |
| 110 | 0.0136 |
| 111 | 0.007 |
| 113 | >10 |
| 114 | >10 |
| 115 | 0.127 |
| 116 | 0.012 |
| 117 | 0.014 |
| 119 | 0.0116 |
| 120 | 0.012 |
| 121 | 0.0118 |
| 123 | 6.253 |
| 124 | 1.013 |
| 125 | 0.005 |

What is claimed:

1. A compound of the formula 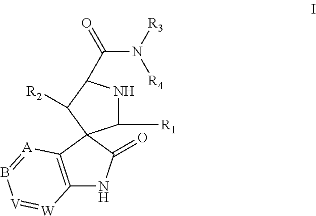
wherein 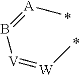
is selected from the group consisting of 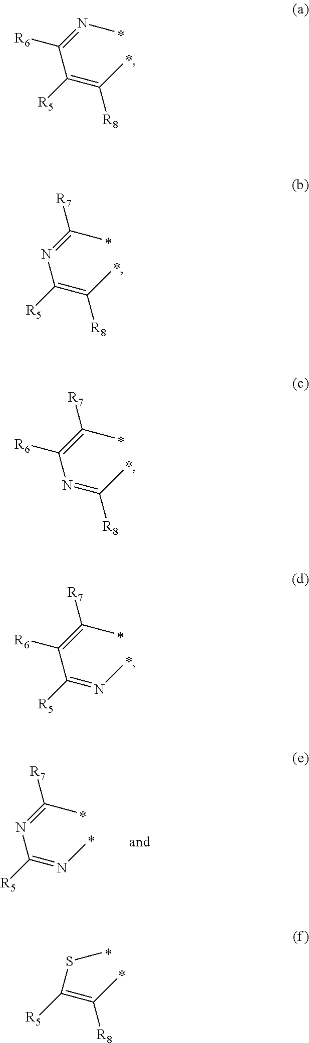
wherein in the case of (f) A is a bond;
R₅ is selected from the group consisting of H, F, Cl, Br, I, cyano, nitro, ethynyl, cyclopropyl, methyl, ethyl, isopropyl, vinyl and methoxy;
R₆ is selected from the group consisting of H, F, Cl, methyl;
R₇ is selected from the group consisting of H, F, Cl, methyl;
R₈ is selected from the group consisting of H, F, Cl, methyl;
R₁ and R₂ are independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R₃ and R₄ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6
or a pharmaceutically acceptable salt thereof.

2. A compound of the formula 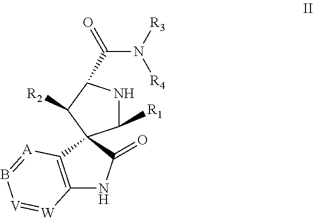
wherein 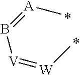
is selected from the group consisting of 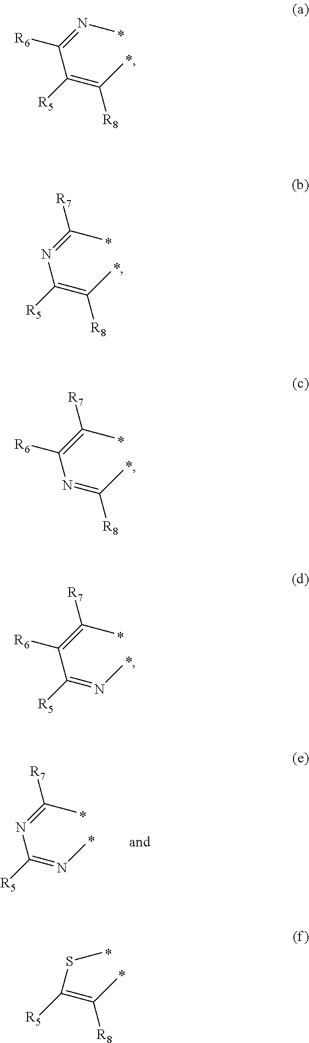
wherein in the case of (f) A is a bond;
R₅ is selected from the group consisting of H, F, Cl, Br, I, cyano, nitro, ethynyl, cyclopropyl, methyl, ethyl, isopropyl, vinyl and methoxy;
R₆ is selected from the group consisting of H, F, Cl and methyl;
R₇ is selected from the group consisting of H, F, Cl and methyl;
R₈ is selected from the group consisting of H, F, Cl and methyl;
R₁ and R₂ are independently selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R₃ and R₄ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —COR′, —SOR′ and SO₂R′
wherein R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle or substituted hetereocycle or R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle;
m, n and p are independently 0 to 6
or a pharmaceutically acceptable salt thereof.

3. The compound of claim 2 wherein R₅ is F, Cl or Br.

4. The compound of claim 3 wherein R₆, R₇, R₈ are all hydrogen.

5. The compound of claim 4 wherein R₂ is selected from the group consisting of aryl, aryl substitued with Cl, F or Br, and heteroaryl optionally substituted with H, F, Cl or Br.

6. The compound of claim 5 wherein R₁ is a substituted lower alkyl of the formula 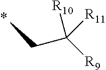
where R₉ and R₁₀ are both methyl, or alternatively, R₉ and R₁₀ together with the carbon to which they are attached form a ring selected from cyclopropyl, cyclobutyl, cyclopentyl or acyclohexyl;
R₁₁ is (CH₂)_{q}—R₁₂, where q is 0, 1 or 2 and
R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle.

7. The compound of claim 6 wherein one of R₃ and R₄ is hydrogen, and the other is (CH₂)ₙ—R′, n is 0 or 1 and R′ is aryl, substituted aryl, hetereoaryl, substituted heteroaryl, hetereocycle or substituted heterocycle.

8. The compound of claim 7 wherein
R₅ is selected from F, Cl or Br;
R₆, R₇, R₈ are hydrogen;
R₂ is selected from the group consisting of aryl, aryl substitued with Cl or F or Br, and heteroaryl optionally substituted with H, F or Cl or Br;
R₁ is a substituted lower alkyl of the formula 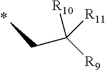
where R₉ and R₁₀ are both methyl, or alternatively, R₉ and R₁₀ together with the carbon to which they are attached form a ring selected from cyclopropyl, cyclobutyl, cyclopentyl or acyclohexyl; R₁₁ is (CH₂)ₙ—R₁₂, where q is 0, 1 or 2;
R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
one of R₃ and R₄ is hydrogen, and the other is (CH₂)ₙ—R′;
n is 0 or 1 and
R′ is selected from aryl, substituted aryl, hetereoaryl, substituted heteroaryl, hetereocycle or substituted heterocycle.

9. A compound of the formula 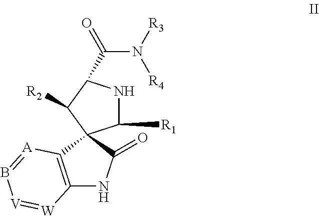
wherein 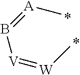
is selected from the group consisting of 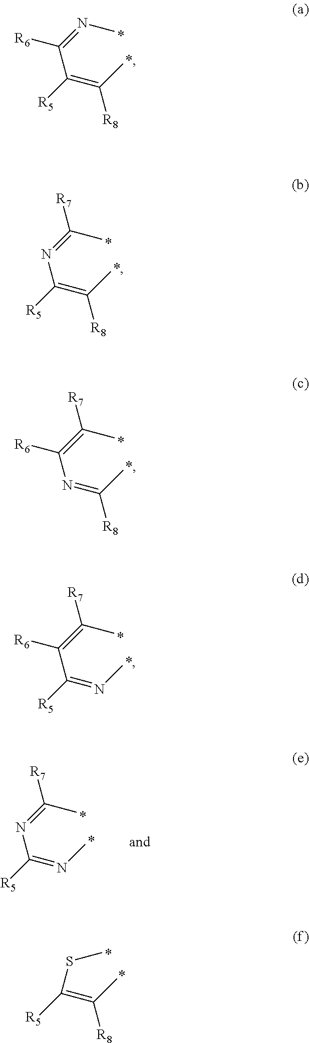
R₅ is selected from F, Cl or Br;
R₆, R₇, R₈ are hydrogen;
R₂ is selected from the group consisting of 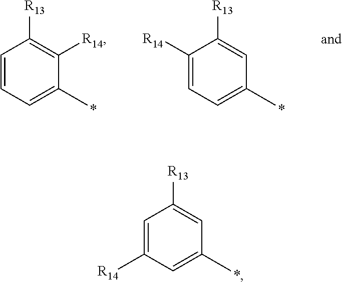
wherein
R₁₃ is F, Cl or Br;
R₁₄ is H or F;
R₁ is a substituted lower alkyl of the formula 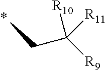
where R₉ and R₁₀ are both methyl, or alternatively, R₉ and R₁₀ together with the carbon to which they are attached form a ring selected from cyclopropyl, cyclobutyl, cyclopentyl or acyclohexyl;
R₁₁ is (CH₂)_{q}—R₁₂, where q is 0, 1 or 2;
R₁₂ is selected from the group consisting of hydrogen, hydroxyl, lower alkyl, substituted lower alkyl, lower alkoxy, substituted lower alkoxy, aryl, substituted aryl, hetereoaryl, substituted heteroaryl, hetereocycle and substituted heterocycle;
one of R₃ and R₄ is hydrogen, and the other is (CH₂)ₙ—R′;
n is 0 or 1;
R′ is selected from aryl, substituted aryl, hetereoaryl, substituted heteroaryl, hetereocycle or substituted heterocycle
or a pharmaceutically acceptable salt thereof.

10. A compound of claim 2 selected from the group consisting of
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
rac-(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid;
rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoate;
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)benzoic acid;
methyl rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoate and
rac-4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-2-methoxybenzoic acid.

11. A compound of claim 2 selected from the group consisting of
rac-(2S,3R,4S,5R)-N-(4-carbamoyl-3-methoxyphenyl)-4-(3-chloro-2-fluorophenyl)-2-neopentyl-2′-oxo-1′,2′-dihydrospiro[pyrrolidine-3,3′-pyrrolo[2,3-b]pyridine]-5-carboxamide;
methyl rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoic acid;
methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoate and rac-4-((2S,3S,4S,5R)-4-(3-chloro-2-fluorophenyl)-6′-methyl-2-neopentyl-2′-oxo-1′,2′-dihydrospiro[pyrrolidine-3,3′-pyrrolo[3,2-b]pyridine]-5-ylcarboxamido)benzoic acid.

12. A compound of claim 2 selected from the group consisting of
methyl rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
rac-4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoic acid;
chiral methyl 4-((2S,3R,4S,5R)-4-(3-chloro-2-fluorophenyl)-2-neopentyl-2′-oxo-1′,2′-dihydrospiro[pyrrolidine-3,3′-pyrrolo[2,3-b]pyridine]-5-ylcarboxamido)-3-methoxybenzoate;
chiral 4-((2S,3R,4S,5R)-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2S,3R,4S,5R)-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
methyl rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
rac-(2S,3S,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-5′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
methyl rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-7′-methyl-2-neopentyl-2′-oxo-1′,2′-dihydrospiro[pyrrolidine-3,3′-pyrrolo[3,2-b]pyridine]-5-ylcarboxamido)-3-methoxybenzoic acid and
chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate.

13. A compound of claim 2 selected from the group consisting of
chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoate;
chiral 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
chiral 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxybenzoic acid;
chiral(2R,3S,4R,5S)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-carboxamide;
chiral 4-((2S,3S,4S,5R)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
chiral 4-((2R,3R,4R,5S)-6′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid;
chiral methyl 4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
chiral methyl 4-((2R,3S,4R,5S)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)benzoate;
methyl rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoate;
rac-4-{(2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido}-3-methoxybenzoic acid;
rac-(2S,3S,4R,5R)-6′-chloro-4-(3-chlorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide and
rac-(2S,3S,4R,5R)-N-(4-carbamoyl-2-methoxyphenyl)-6′-chloro-4-(3-chlorophenyl)-2-neopentyl-2′-oxo-1′,2′-dihydrospiro[pyrrolidine-3,3′-pyrrolo[3,2-b]pyridine]-5-carboxamide.

14. A compound of claim 2 selected from the group consisting of
methyl rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate;
methyl rac-4-{(2S,3R,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoate;
rac-4-{(2S,3S,4S,5R)-4-(3-bromo-2-fluorophenyl)-6′-chloro-2-neopentyl-2′-oxo-1′,2′-dihydrospiro[pyrrolidine-3,3′-pyrrolo[3,2-b]pyridine]-5-ylcarboxamido}-3-methoxybenzoic acid;
rac-(2S,3S,4S,5R)-4-(3-bromo-2-fluorophenyl)-6′-chloro-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-bromo-2-fluorophenyl)-N-(4-carbamoyl-2-methoxyphenyl)-6′-chloro-2-neopentyl-2′-oxo-1′,2′-dihydrospiro[pyrrolidine-3,3′-pyrrolo[3,2-b]pyridine]-5-carboxamide;
rac-2-(4-((2S,3S,4R,5R)-6′-chloro-4-(3-chlorophenyl)-2-neopentyl-2′-oxo-1′,2′-dihydrospiro[pyrrolidine-3,3′-pyrrolo[3,2-b]pyridine]-5-ylcarboxamido)-3-methoxyphenoxy)ethyl acetate;
rac-(2S,3S,4R,5R)-6′-chloro-4-(3-chlorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-bromo-2-fluorophenyl)-6′-chloro-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
chiral(2S,3S,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-6′-bromo-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid and
rac-(2S,3S,4S,5R)-6′-bromo-4-(3-chloro-2-fluorophenyl)-N-(4-cyano-2-methoxyphenyl)-2-neopentyl-2′-oxo-1′,2′-dihydrospiro[pyrrolidine-3,3′-pyrrolo[3,2-b]pyridine]-5-carboxamide.

15. A compound of claim 2 selected from the group consisting of
rac-(2S,3S,4S,5R)-6′-bromo-N-(4-carbamoyl-2-methoxyphenyl)-4-(3-chloro-2-fluorophenyl)-2-neopentyl-2′-oxo-1′,2′-dihydrospiro[pyrrolidine-3,3′-pyrrolo[3,2-b]pyridine]-5-carboxamide;
rac-(2S,3S,4S,5R)-6′-bromo-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-b]pyridine]-5-carboxamide;
methyl rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoate;
rac-4-((2S,3S,4S,5R)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-ylcarboxamido)-3-methoxybenzoic acid;
rac-(2S,3S,4S,5R)-4-(3-chloro-2-fluorophenyl)-N-(4-cyano-2-methoxyphenyl)-6′-fluoro-2-neopentyl-2′-oxo-1′,2′-dihydrospiro[pyrrolidine-3,3′-pyrrolo[3,2-b]pyridine]-5-carboxamide;
rac-(2S,3S,4S,5R)-N-(4-carbamoyl-2-methoxyphenyl)-4-(3-chloro-2-fluorophenyl)-6′-fluoro-2-neopentyl-2′-oxo-1′,2′-dihydrospiro[pyrrolidine-3,3′-pyrrolo[3,2-b]pyridine]-5-carboxamide;
rac-(2S,3S,4S,5R)-4-(3-chloro-2-fluorophenyl)-N-[4-(2-hydroxyethoxy)-2-methoxyphenyl]-6′-fluoro-2-neopentyl-2′-oxo-1′,2′-dihydrospiro[pyrrolidine-3,3′-pyrrolo[3,2-b]pyridine]-5-carboxamide;
rac-(2S,3R,4S,5R)-2′-chloro-4-(3-chloro-2-fluorophenyl)-N-[4-(2-hydroxyethoxy)-2-methoxyphenyl]-2-neopentyl-6′-oxo-6′,7′-dihydrospiro[pyrrolidine-3,5′-pyrrolo[2,3-d]pyrimidine]-5-carboxamide;
methyl rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate;
methyl rac-4-{(2S,3S,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoate;
rac-4-{(2S,3R,4S,5R)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-ylcarboxamido}-3-methoxybenzoic acid;
rac-(2S,3R,4S,5R)-N-(4-carbamoyl-2-methoxyphenyl)-2′-chloro-4-(3-chloro-2-fluorophenyl)-2-neopentyl-6′-oxo-6′,7′-dihydrospiro[pyrrolidine-3,5′-pyrrolo[2,3-d]pyrimidine]-5-carboxamide and
methyl rac-4-((2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate.

16. A compound of claim 2 selected from the group consisting of
methyl rac-4-((2S,3S,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido)-3-methoxybenzoate;
rac-4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid;
chiral 4-{(2S,3R,4R,5R)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-ylcarboxamido}-3-methoxybenzoic acid;
rac-(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
chiral(2S,3R,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
rac-(2S,3S,4R,5R)-2′-chloro-4-(3-chloro-2-fluorophenyl)-N-(4-cyano-2-methoxyphenyl)-2-neopentyl-5′-oxo-4′,5′-dihydrospiro[pyrrolidine-3,6′-thieno[3,2-b]pyrrole]-5-carboxamide;
rac-(2S,3S,4R,5R)-N-(4-carbamoyl-2-methoxyphenyl)-2′-chloro-4-(3-chloro-2-fluorophenyl)-2-neopentyl-5′-oxo-4′,5′-dihydrospiro[pyrrolidine-3,6′-thieno[3,2-b]pyrrole]-5-carboxamide;
rac-(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide and
chiral(2S,3R,4R,5R)-2′-chloro-4-(3-chloro-2-fluorophenyl)-N-(4-(2-hydroxyethoxy)-2-methoxyphenyl)-2-neopentyl-5′-oxo-4′,5′-dihydrospiro[pyrrolidine-3,6′-thieno[3,2-b]pyrrole]-5-carboxamide.

17. A compound of claim 2 selected from the group consisting of
methyl rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-ylcarboxamido)-3-methoxybenzoic acid,
methyl rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-hydroxy-2-neopentyl-2′-oxo-1′,2′-dihydrospiro[pyrrolidine-3,3′-pyrrolo[3,2-c]pyridine]-5-ylcarboxamido)-3-methoxybenzoic acid,
rac-(2S,3R,4S,5R)-N-(4-carbamoyl-2-methoxyphenyl)-4-(3-chloro-2-fluorophenyl)-6′-methoxy-2-neopentyl-2′-oxo-1′,2′-dihydrospiro[pyrrolidine-3,3′-pyrrolo[3,2-c]pyridine]-5-carboxamide,
rac-2-(4-((2S,3R,4S,5R)-6′-chloro-4-(3-chloro-2-fluorophenyl)-2-neopentyl-2′-oxo-1′,2′-dihydrospiro[pyrrolidine-3,3′-pyrrolo[3,2-c]pyridine]-5-ylcarboxamido)-3-methoxyphenoxy)ethyl acetate,
rac-(2S,3R,4S,5R)-6′-chloro-4-(3-chloro-2-fluorophenyl)-N-(4-(2-hydroxyethoxy)-2-methoxyphenyl)-2-neopentyl-2′-oxo-1′,2′-dihydrospiro[pyrrolidine-3,3′-pyrrolo[3,2-c]pyridine]-5-carboxamide,
methyl rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoate,
rac-4-((2S,3R,4S,5R)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-ylcarboxamido)-3-methoxybenzoic acid,
rac-(2S,3R,4S,5R)-N-(4-carbamoyl-2-methoxyphenyl)-4-(3-chloro-2-fluorophenyl)-6′-methyl-2-neopentyl-2′-oxo-1′,2′-dihydrospiro[pyrrolidine-3,3′-pyrrolo[3,2-c]pyridine]-5-carboxamide,
methyl rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoate,
rac-4-((2S,3S,4S,5R)-7′-chloro-4-(3-chloro-2-fluorophenyl)-2-neopentyl-2′-oxo-1′,2′-dihydrospiro[pyrrolidine-3,3′-pyrrolo[3,2-b]pyridine]-5-ylcarboxamido)-3-methoxybenzoic acid and
rac-(2S,3R,4S,5R)-N-(4-carbamoyl-2-methoxyphenyl)-6′-chloro-4-(3-chloro-2-fluorophenyl)-2-neopentyl-2′-oxo-1′,2′-dihydrospiro[pyrrolidine-3,3′-pyrrolo[2,3-b]pyridine]-5-carboxamide.

18. A pharmaceutical composition comprising a compound of claim 2, or a pharmaceutically acceptable salt thereof, as an active ingredient together with a pharmaceutically acceptable carrier or excipient.
